(19) United States
(12) Patent Application Publication
(10) Pub. No.: US 2017/0342077 A1
(43) Pub. Date: Nov. 30, 2017

(54) DIAZENE DIRECTED MODULAR SYNTHESIS OF COMPOUNDS WITH QUATERNARY CARBON CENTERS

(71) Applicants: Mohammad MOVASSAGHI, Arlington, MA (US); Petra LINDOVSKA, Cambridge, MA (US)

(72) Inventors: Mohammad MOVASSAGHI, Arlington, MA (US); Petra LINDOVSKA, Cambridge, MA (US)

(21) Appl. No.: 15/604,508

(22) Filed: May 24, 2017

Related U.S. Application Data
(60) Provisional application No. 62/340,948, filed on May 24, 2016.

(51) Int. Cl. C07D 487/04; A61K 31/404

(52) U.S. Cl. CPC C07D 487/04; A61K 31/404

(57) ABSTRACT

Diazene-directed modular synthesis is described for the preparation Csp2-Csp3 and Csp3-Csp3 linkages where one or more stereogenic quaternary carbon centers are formed. The disclosed methods are directed to the preparation of compounds of Formula (I), or a pharmaceutically acceptable salt, tautomer or stereoisomer thereof, from compounds of Formula (II):

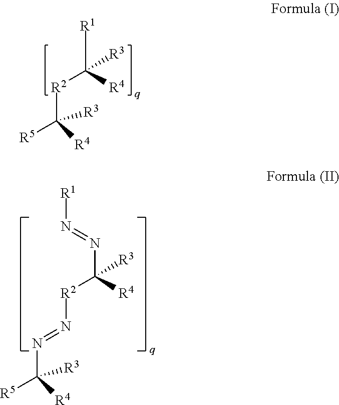

wherein R¹-R⁵ and q are as defined independently for each occurrence herein. A wide variety of compounds can be accessed in this manner, including oligocyclotryptamines, where the stereochemistry of each subunit is beneficially secured before fragment coupling.

### CROSS-REFERENCE TO RELATED APPLICATION

This application claims the benefit of and priority to U.S. Provisional Application No. 62/340,948, filed on May 24, 2016 and entitled “DIAZENE DIRECTED MODULAR SYNTHESIS OF OLIGOCYCLOTRYPTAMINES,” the disclosure of which is hereby incorporated by reference in its entirety for all purposes.

### STATEMENT OF GOVERNMENT INTEREST

This invention was made with Government support under Grant No. R01 GM089732 awarded by the National Institutes of Health. The Government has certain rights in the invention.

### BACKGROUND

The establishment of quaternary centers, particularly with defined stereochemistry, remains a challenge in the field of organic synthesis. As a variety of natural products, bioactive molecules, and other analogs possess such a feature, novel methods and approaches are necessary that improve access to these compounds.

### BRIEF SUMMARY

Various inventive embodiments disclosed herein are generally directed to formation of quaternary centers as shown below using novel diazene-directed synthesis methodologies and processes, wherein X, R, R′, R″, and EWG (electron withdrawing group) are each as defined and discussed herein.

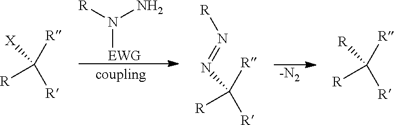

In one embodiment, the present disclosure provides a method of preparing compounds of Formula (I), or a pharmaceutically acceptable salt, tautomer or stereoisomer thereof comprising reacting a compound of Formula (II) and thereby extruding dinitrogen to provide a compound of Formula (I):

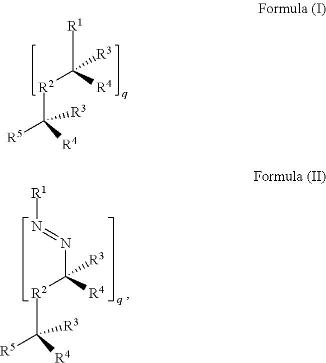

wherein
R¹ is tertiary alkyl, alkenyl, aryl, heteroaryl, carbocyclyl, or heterocyclyl or at least one moiety of structure:

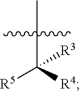

and
R², R³, R⁴, and R⁵ are each occurrence, each independently selected from alkyl, aryl, heteroaryl, carbocyclyl, or heterocyclic, wherein any two of R³, R⁴, and R⁵ taken together with the carbon atoms to which they are attached form a C₅₋₁₄ membered saturated, unsaturated, or aromatic carbocyclic ring or a C₅₋₁₄ membered saturated, unsaturated, or aromatic heterocyclic ring; and wherein any tertiary alkyl, aryl, heteroaryl, carbocyclyl, or heterocyclyl, C₅₋₁₄ membered saturated, unsaturated, or aromatic carbocyclic ring or a C₅₋₁₄ membered saturated, unsaturated, or aromatic heterocyclic ring can be further substituted with one or more halogen, alkyl, heteroaryl, carbocyclyl, heterocyclyl, C₃₋₁₄ membered saturated, unsaturated, or aromatic carbocyclic, or C₃₋₁₄ membered saturated, unsaturated, or aromatic heterocyclic rings; and q is an integer of from 0-8.

In another embodiment, the method as disclosed herein is useful for the preparation of compounds of Formula (I):

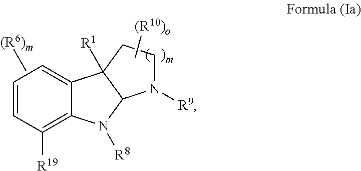

wherein
R¹ is tertiary alkyl, alkenyl, aryl, heteroaryl, carbocyclyl, heterocyclyl,

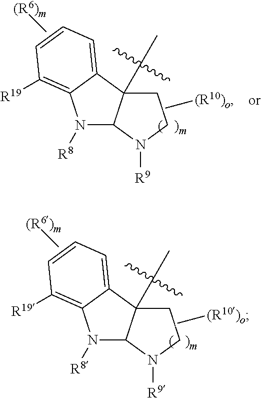

R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ groups taken together with the carbon atoms to which they are attached form an aryl, heteroaryl, carbocyclic, or heterocyclic ring;
R⁸, R⁹, R^{8′} and R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R¹⁰ groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹¹, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R¹⁹ and R^{19′} are each independently selected from H, tertiary alkyl, alkenyl, aryl, heteroaryl, carbocyclyl, heterocyclyl,

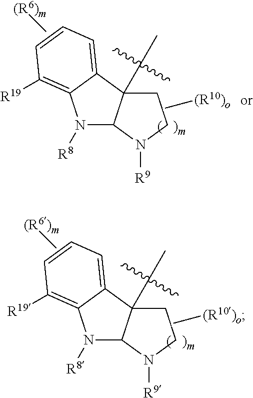

R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

In specific embodiments, the compounds of Formula (I) prepared by the disclosed methods are:

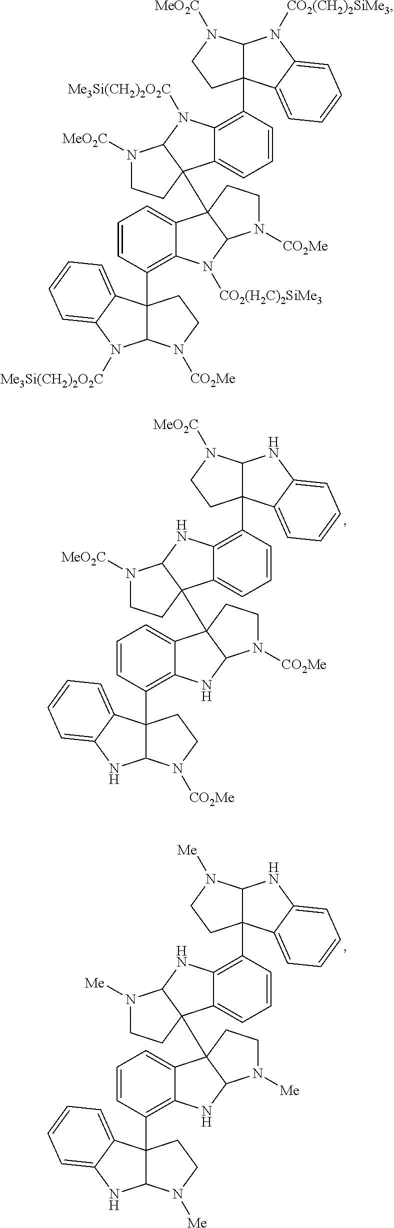

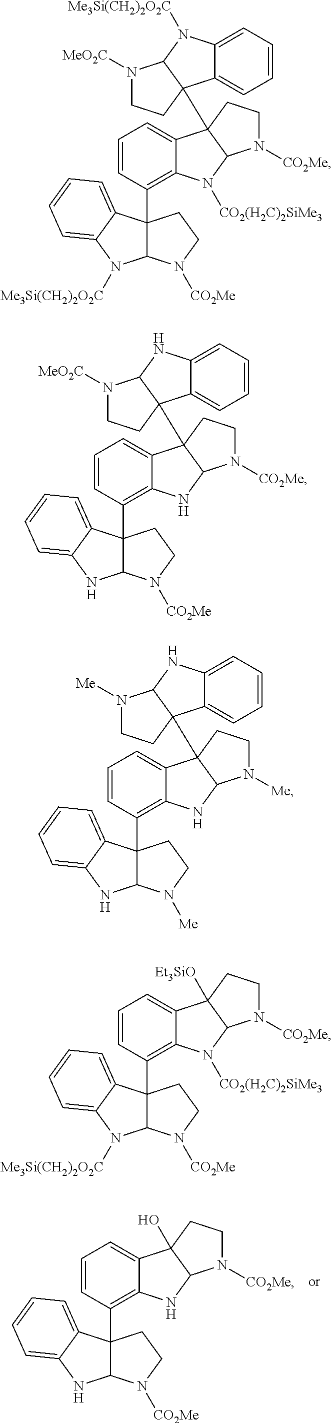

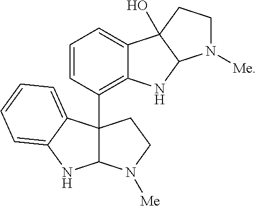 [SMILES: CN1CCC2(O)c3cccc(C45CCN(C)C4Nc4ccccc45)c3NC12]

### BRIEF DESCRIPTION OF THE FIGURES

The skilled artisan will understand that the drawings are primarily for illustrative purposes and are not intended to limit the scope of the inventive subject matter described herein.

FIG. 1 shows the ¹H NMR spectrum of the sulfamide-diazene intermediate of (−)-quadrigemine C.

FIG. 1A shows the iterative and modular synthesis of oligocyclotryptamines from various building blocks, according to some embodiments of the disclosure.

FIG. 2 shows the ¹H NMR spectrum of the tri-diazene intermediate of (−)-quadrigemine C.

FIG. 3 shows the ¹H NMR spectrum of (−)-quadrigemine C.

FIG. 4 is the mass spectral data confirming the formation of (−)-quadrigemine C.

FIG. 5 shows the ¹H NMR spectrum of (−)-hodgkinsine.

FIG. 6 is the mass spectral data confirming the formation of (−)-hodgkinsine.

### DETAILED DESCRIPTION

In the following description, certain specific details are set forth in order to provide a thorough understanding of various embodiments. However, one skilled in the art will understand that the invention can be practiced without these details. In other instances, well-known structures have not been shown or described in detail to avoid unnecessarily obscuring descriptions of the embodiments.

All definitions, as defined and used herein, should be understood to control over dictionary definitions, definitions in documents incorporated by reference, and/or ordinary meanings of the defined terms. Use of flow diagrams is not meant to be limiting with respect to the order of operations performed for all embodiments. The indefinite articles “a” and “an,” as used herein in the specification and in the claims, unless clearly indicated to the contrary, should be understood to mean “at least one.”

Reference throughout this specification to “one embodiment” or “an embodiment,” etc. means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It is to be understood that the phrase “each occurrence” and “each independently selected from” as used in the specification, and in the claims means, for example, that two or more R^{x} groups can be non-equivalent selections when appearing together in one compound or formula. Where the phrase “R^{x} and R^{y} are each independently selected from” is used interchangeably, it is intended to have the same meaning as “R^{x} and R^{y} are each occurrence, each independently selected from.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

“Alkyl” or “alkyl group” refers to a fully saturated, straight or branched hydrocarbon chain radical, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical. Alkylenes comprising any number of carbon atoms from 1 to 12 are included. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Examples of C₁-C₃ alkyl includes methyl, ethyl, n-propyl, and i-propyl. Examples of C₁-C₄ alkyl includes methyl, ethyl, n-propyl, i-propyl, n-butyl, i-butyl, and sec-butyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl groups comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alkenyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetyl”) moiety. Alkylcarbonyl groups can also be referred to as “Cw-Cz acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” refers to a radical of the formula —R_{b}-R_{c} where R_{b} is an alkylene, alkenylene or alkynylene group as defined above and R is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls, cycloalkyl, cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}-R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above that is substituted by one or more halo radicals, as defined above, e.g., 1-fluoropropynyl, 1-fluorobutynyl, and the like. Unless stated otherwise specifically in the specification, a haloalkenyl group can be optionally substituted.

“Heterocyclyl,” “heterocyclic ring” or “heterocycle” refers to a stable 3- to 20-membered non-aromatic ring radical which consists of two to twelve carbon atoms and from one to six heteroatoms selected from the group consisting of nitrogen, oxygen and sulfur. Heterocyclyl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heterocyclylalkyl” refers to a radical of the formula —R_{b}-Rₑ where R_{b} is an alkylene, alkenylene, or alkynylene chain as defined above and Rₑ is a heterocyclyl radical as defined above, and if the heterocyclyl is a nitrogen-containing heterocyclyl, the heterocyclyl can be attached to the alkyl, alkenyl, alkynyl radical at the nitrogen atom. Unless stated otherwise specifically in the specification, a heterocyclylalkyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in this disclosure, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}-R_{f} where R_{b} is an alkylene, alkenylene, or alkynylene chain as defined above and R_{f} is a heteroaryl radical as defined above. Unless stated otherwise specifically in the specification, a heteroarylalkyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}C(═O)ORₕ, —NR_{g}SO₂Rₕ, —OC(═O)NR_{g}Rₕ, —OR_{g}—SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

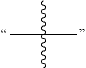

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

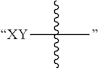

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

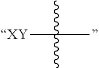

infers that when R³ is “XY”, the point of attachment bond is the same bond as the bond by which R³ is depicted as being bonded to CH₃.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring can be replaced with a nitrogen atom.

“Optional” or “optionally” means that the subsequently described event of circumstances can or cannot occur, and that the description includes instances where said event or circumstance occurs and instances in which it does not. For example, “optionally substituted aryl” means that the aryl radical can or cannot be substituted and that the description includes both substituted aryl radicals and aryl radicals having no substitution.

Total synthesis refers to the complete chemical synthesis of a complex molecule, typically a natural product or a structurally similar analog or derivative thereof, starting from commercially available precursor compounds. It is often desirable to perform total syntheses in a “convergent” manner, where efficiency and overall chemical yield are improved by synthesizing several complex individual components in stage one, followed by combination of the components in a subsequent stage to yield a more advanced compound or final product. While convergent synthetic methods are sometimes desirable, for complex molecular frameworks such as cyclotryptamines and oligocyclotryptamines, there can be many different possible linear or convergent approaches. The success of any particular approach is highly unpredictable.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present invention is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present invention includes tautomers of any said compounds.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Crystallization is a method commonly used to isolate a reaction product, for example one of the compounds disclosed herein, in purified form. Often, crystallization produces a solvate of the compound of the invention. As used herein, the term “solvate” refers to an aggregate that comprises one or more molecules of a compound of the invention with one or more molecules of solvent, typically in co-crystallized form. The solvent can be water, in which case the solvate can be a hydrate. Alternatively, the solvent can be an organic solvent. Thus, the compounds of the present invention can exist as a hydrate, including a monohydrate, dihydrate, hemihydrate, sesquihydrate, trihydrate, tetrahydrate and the like, as well as the corresponding solvated forms. The compound of the invention can be true solvates, while in other cases, the compound of the invention can merely retain adventitious water or be a mixture of water plus some adventitious solvent.

The chemical naming protocol and structure diagrams used herein are a modified form of the I.U.P.A.C. nomenclature system, using the ACD/Name Version 9.07 software program, ChemDraw Ultra Version 11.0.1 and/or ChemDraw Ultra Version 14.0 and/or ChemDraw Professional 16.0.0.82 software naming program (CambridgeSoft), or the like. For complex chemical names employed herein, a substituent group is named before the group to which it attaches. For example, cyclopropylethyl comprises an ethyl backbone with cyclopropyl substituent. Except as described below, all bonds are identified in the chemical structure diagrams herein, except for some carbon atoms, which are assumed to be bonded to sufficient hydrogen atoms to complete the valency.

Following below are more detailed descriptions of various concepts related to, and embodiments of, inventive methods for Diazene Directed Modular Synthesis of Compounds with Quaternary Carbon Centers. It should be appreciated that various concepts introduced above and discussed in greater detail below can be implemented in any of numerous ways, as the disclosed concepts are not limited to any particular manner of implementation. Examples of specific implementations and applications are provided primarily for illustrative purposes.

In one embodiment, the present disclosure provides method of preparing compounds of Formula (I), or a pharmaceutically acceptable salt, tautomer or stereoisomer thereof, comprising reacting a compound of Formula (II), and thereby extruding dinitrogen to provide a compound of Formula (I):

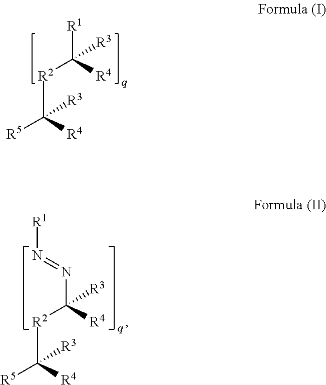

wherein R¹ is tertiary alkyl, alkenyl, aryl, heteroaryl, carbocyclyl, or heterocyclyl or at least one moiety of structure:

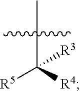

R², R³, R⁴, and R⁵ are each occurrence, each independently selected from tertiary alkyl, aryl, heteroaryl, carbocyclyl, or heterocyclic, wherein any two of R³, R⁴, and R⁵ taken together with the carbon atoms to which they are attached form a C₅₋₁₄ membered saturated, unsaturated, or aromatic carbocyclic ring or a C₅₋₁₄ membered saturated, unsaturated, or aromatic heterocyclic ring; and wherein any tertiary alkyl, aryl, heteroaryl, carbocyclyl, or heterocyclyl, C₅₋₁₄ membered saturated, unsaturated, or aromatic carbocyclic ring or a C₅₋₁₄ membered saturated, unsaturated, or aromatic heterocyclic ring can be further substituted with one or more halogen, alkyl, heteroaryl, carbocyclyl, heterocyclyl, C₃₋₁₄ membered saturated, unsaturated, or aromatic carbocyclic, or C₃₋₁₄ membered saturated, unsaturated, or aromatic heterocyclic rings; and q is an integer from 0-8.

In some embodiments, q is 1, 2, 3, 4, 5, 6, 7, or 8. In other embodiments, q is an integer from 0 to 1, 0 to 2, 0 to 3, 0 to 4, 0 to 5, 0 to 6, or 0 to 7.

In various embodiments, the formation of the compound of Formula (I) from the compound of Formula (II) occurs via a radical recombination reaction. In another embodiment, the stereochemical configuration of the compound of Formula (II) is retained in the compound of Formula (I) following the reaction. In other embodiments, the radical recombination reaction results in a Csp3-Csp3 bond or a Csp3-Csp2-bond. In a specific embodiment, the bond formed is a Csp3-Csp2 bond. In another specific embodiment, the bond formed is a Csp3-Csp2 bond. The radical recombination reaction can be initiated under photolysis conditions by irradiating the compounds of Formula (II). In some embodiments, the irradiation is carried out in a photoreactor. In other embodiments, the photoreactor is equipped with 1 to about 20 lamps operating at a wavelength λ from about 250 nm to about 400 nm. Various wavelengths of light may be suitable including 251 nm, 252 nm, 253 nm, 254 nm, 255 nm, 256 nm, 257 nm, 258 nm, 259 nm, 260 nm, 261 nm, 262 nm, 263 nm, 264 nm, 265 nm, 266 nm, 267 nm, 268 nm, 269 nm, 270 nm, 271 nm, 272 nm, 273 nm, 274 nm, 275 nm, 276 nm, 277 nm, 278 nm, 279 nm, 280 nm, continuing up to about 400 nm, inclusive of all values therebetween. In more specific embodiments, the wavelength λ is 300 nm or 380 nm.

In other embodiments, the radical recombination reaction can be initiated under thermal conditions, such as described in Nelsen, S. F.; Bartlett, P. D. JACS 1966, 88, 137-143 and 143-149, and Engel, P. S.; Pan, L.; Ying, Y.; Alemany, L. B. JACS 2001, 123, 3706-3715, each of which is herein expressly incorporated by reference. In some embodiments, the thermal reaction is carried out from about 60° C. to about 250° C. In other embodiments, the thermal reaction is carried out from 100 OC to about 150° C. In a specific embodiment, the thermal reaction is carried out at 120° C. In another embodiment, the radical recombination reaction can be initiated under flash vacuum pyrolysis conditions.

In another embodiment, the present method as disclosed herein is useful in preparing compound of Formula (I), wherein compounds of Formula (I) are:

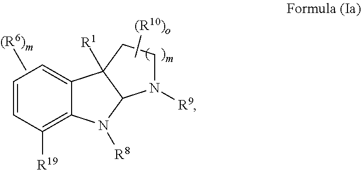

and
wherein R¹ is tertiary alkyl, alkenyl, aryl, heteroaryl, carbocyclyl, heterocyclyl,

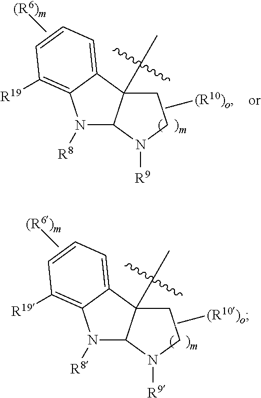

R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R⁹, R^{8′}, and R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², —SR¹¹, —S(═O)R¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)R¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹; R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R¹⁹ and R^{19′} are each independently selected from H, tertiary alkyl, alkenyl, aryl, heteroaryl, carbocyclyl, heterocyclyl,

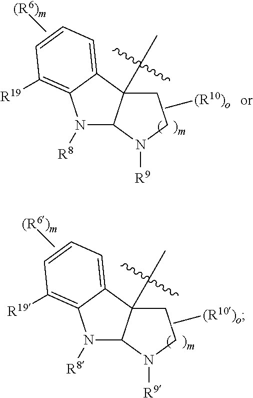

R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

In another embodiment, the present method as disclosed herein is useful in preparing compounds of Formula (Ia):

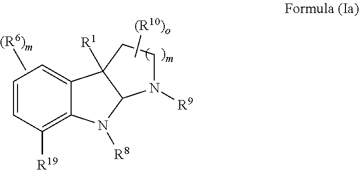

wherein R¹ is further defined as

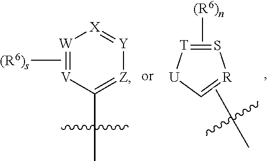

and
wherein V, W, X, Y, and Z are each independently selected from —CH or N;
R, S, and T are each independently selected from —CH or N; and
U is O, S, or NR¹¹; wherein R¹¹ is independently selected from H, C₁-C₁₂ alkyl, C₂-C₁₂ alkenyl, C₂-C₁₂ alkynyl, aryl, heteroaryl, carbocyclyl, or heterocyclyl; and
n is an integer from 0-4; and
s is an integer from 0-5.

In a more specific embodiment, the present method as disclosed herein is useful in preparing compounds of Formula (I), wherein compounds of Formula (I) are:

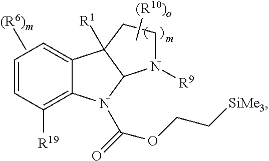

and
wherein R¹⁹ is H, tertiary alkyl, alkenyl, aryl, heteroaryl, carbocyclyl, heterocyclyl,

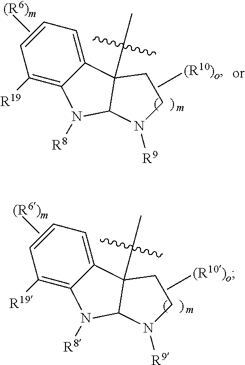

and
R⁸ and R^{8′} are —C(═O)O(CH₂)₂SiMe₃; and
R⁶, R^{6′}, R⁹, R^{9′}, R¹⁰, R^{10′}, and R^{19′} are defined as above for Formula (Ia).

In another embodiment, the method as presently disclosed herein is useful in preparing compounds of Formula (IIa):

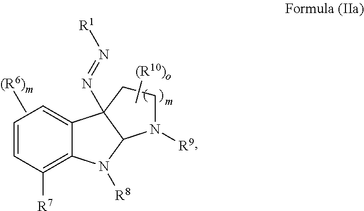

wherein R¹ is tertiary alkyl, alkenyl, aryl, heteroaryl, carbocyclyl, heterocyclyl,

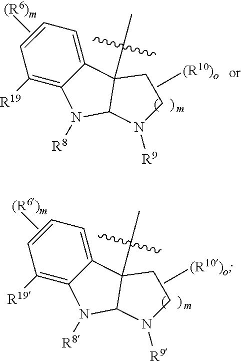

R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring; R⁷ and R^{7′} are each independently selected from H, —N₃, —N(R⁵)NH₂, —NHR¹⁵,

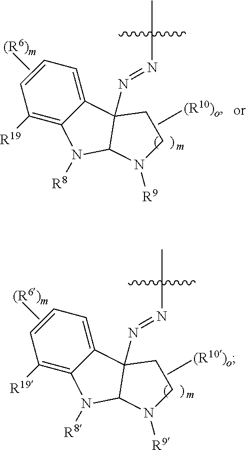

R⁸, R^{8′}, R⁹, and R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹² aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR², —C(═O)CF₃, —C(═O)OR²⁰, —P(═O)R¹³R¹⁴, or, —P(═O)NR¹¹R¹²;
R¹⁹ and R^{19′} are each independently H;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

In another embodiment, the method as presently disclosed herein is useful in preparing compounds of Formula (IIa):

wherein R¹ is,

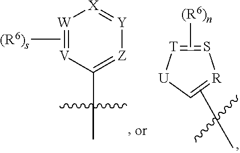

and
wherein V, W, X, Y, and Z are each independently selected from —CH or N;
R, S, and T are each independently selected from —CH or N;
U is O, S, or NR¹¹; wherein
R¹¹ is independently selected from H, C₁-C₁₂ alkyl, C₂-C₁₂ alkenyl, C₂-C₁₂ alkynyl, —Si(alkyl)₃, —Si(alkyl)₂aryl, Si(aryl)₂alkyl, aryl, heteroaryl, carbocyclyl, or heterocyclyl, and wherein R¹¹ and R¹² taken together with the carbon atoms to which they are attached form an aryl, heteroaryl, carbocyclic, or heterocyclic ring; and
n is an integer from 0-4; and
s is an integer from 0-5; and
wherein R⁶, R⁷, R⁸, R⁹, and R¹⁰ are defined as above for Formula (IIa).

In another embodiment, the present disclosure provides a method of preparing compounds of Formula (II) by reacting compounds of Formula (III) and compounds of Formula

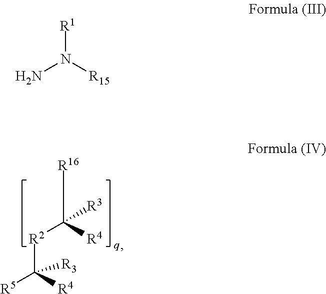

wherein
R¹ is alkenyl, aryl, or heteroaryl; and
R², R³, R⁴, and R⁵ are each occurrence, each independently selected from alkyl, aryl, heteroaryl, carbocyclyl, or heterocyclic, wherein any two of R³, R⁴ and R⁵ taken together with the carbon atoms to which they are attached form a C₅₋₁₄ membered saturated, unsaturated, or aromatic carbocyclic ring or a C₅₋₁₄ membered saturated, unsaturated, or aromatic heterocyclic ring; and wherein any tertiary alkyl, aryl, heteroaryl, carbocyclyl, or heterocyclyl, C₅₋₁₄ membered saturated, unsaturated, or aromatic carbocyclic ring or a C₅₋₁₄ membered saturated, unsaturated, or aromatic heterocyclic ring can be further substituted with one or more halogen, alkyl, heteroaryl, carbocyclyl, heterocyclyl, C₃₋₁₄ membered saturated, unsaturated, or aromatic carbocyclic, or C₃₋₁₄ membered saturated, unsaturated, or aromatic heterocyclic rings;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R², —C(═O)O(CH₂)ᵣR²⁰, —C(═O)CF₃, —C(═O)OR²⁰, —P(═O)R¹³R¹⁴, or, —P(═O)NR¹¹R¹²;
R¹⁶ is I, Br, Cl, —OH, —OSO₂CF₃, —OS(O)₂R¹³, —OP(═O)R¹³R¹⁴, —OC(═NR¹¹)R², —OC(═NR¹¹)CCl₃, —OR¹¹, or —N₂⁺X⁻, wherein X⁻ is halogen;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2;
q is an integer of from 0-8; and
r is an integer from 1 to 4.

In one embodiment the method comprises an electrophilic activation of a compound of Formula (IV). In another embodiment, the electrophilic activation comprises reaction of the compound of Formula (IV) with a silver (I) salt and a base. In various embodiments, the silver (I) salt can be AgOSO₂CF₃, AgSbF₆, Ag(OSO₂CF₂CF₂CF₂CF₃), AgBF₄, or AgN(SO₂CF₃)₂. In specific embodiments, the silver (I) salt is AgOSO₂CF₃ or AgSbF₆. In other embodiments, the compound of Formula (III) is resistant to oxidation by the silver (I) salt. In other embodiments, the compound of Formula (III) is less prone to oxidation. The presence of an electron withdrawing group renders compounds of Formula (III) resistant to oxidization under the reaction conditions for at least 45 minutes, and typically for a period of up to 6 hours or more. In contrast, a compound such as phenylhydrazine with no electron withdrawing group affords no desired products, as it is decomposed immediately in the presence of the silver (I) salts. In a specific embodiment of a compound of Formula (III), R¹⁵ is —S(═O)ₚR¹³, wherein p is 2 and R¹³ is C₁-C₁₂ alkyl. Accordingly, when R¹⁵ is —S(═O)ₚR¹³, wherein p is 2 and R¹³ is C₁-C₁₂ alkyl, the compound of Formula (II) is formed in one synthetic step.

The new diazene synthesis strategy is facilitated through placement of an electron withdrawing group on the hydrazine that makes the nucleophilic hydrazine compatible with conditions needed for activation of the electrophile. The highlighted examples allow the union of the nucleophile and the electrophile followed by spontaneous loss of the electron withdrawing group to give the diazene.

Other examples of electron withdrawing groups demonstrated in our data provided herein include but are not limited to the use of BOC and trifluoroacetyl group. These groups lead to efficient formation of the nucleophile-electrophile adduct as the hydrazine intermediate. In an embodiment using trifluoroacetyl, a mild deacylation and oxidation leads to the desired diazene.

The methanesulfonyl group shown above does not require a separate step, but this characteristic can be applicable to other variations, embodiments, and implementations:

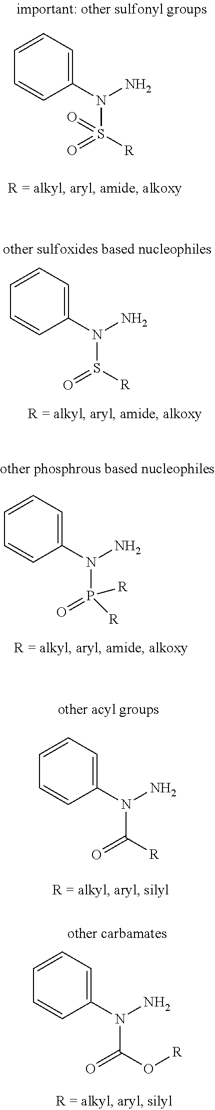

In addition to the methane sulfonamide, this strategy can be applied to arene sulfonamides (e.g., toluenesulfonyl, benzenesulfonyl), and alkyl sulfonyl derivatives along with sulfonamide (RSO₂ vs. R₂NSO₂) or sulfamate (RSO₂ vs. ROSO₂) variations. Use of sulfoxide variations (SO vs. SO₂) is also possible. Additionally, phosphorous based systems can be utilized as well (RSO₂ vs. R₂PO). Such embodiments provide benefits similar to the exemplary reagent class discussed herein.

Other hydrazines including simple amides and carbamates can be utilized, and in some implementations, can be as equally effective as the Boc-carbamate and the TFA-amide that have been demonstrated herein.

Use of these reagent classes can be extended to conditions not necessary involving silver activation. These nucleophiles are excellent substitutes for direct synthesis of diazene through N-alkylation (under a variety of conditions).

These reagent classes stand in contrast to simple hydrazine (ArNHNH₂) since exposure of ArNHNH₂ to promoters such as silver may not be possible due to rapid oxidation of the hydrazine. These hydrazines may be used for simple electrophiles (primary and secondary electrophiles), but for complex and sterically crowded electrophiles (tertiary carbon), technology and methods as disclosed herein is needed.

In yet another embodiment, the method as presently disclosed herein is useful in preparing compounds of Formula (II) from compounds of Formula (III), wherein the compound of Formula (III) is:

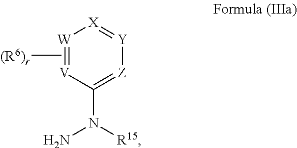

wherein V, W, X, Y, and Z are each independently selected from —CH or N; and
r is an integer from 0 to 5.

In other embodiments, the method as presently disclosed herein is useful in preparing compounds of Formula (II) from compounds of Formula (III), wherein the compound of Formula (III) is:

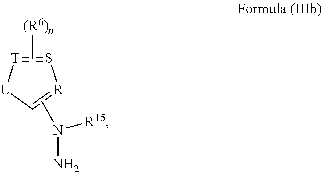

wherein R, S, and T are each independently selected from —CH or N; and
U is O, S, or NR¹¹, wherein R¹¹ is independently selected from H, C₁-C₁₂ alkyl, C₂-C₁₂ alkenyl, C₂-C₁₂ alkynyl, aryl, heteroaryl, carbocyclyl, or heterocyclyl; and
n is an integer from 0 to 4.

In still other various embodiments, the compound of Formula (III) is:

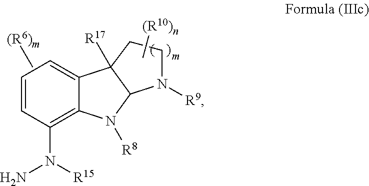

wherein R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R¹⁷ is H, —OH, —OR¹¹, —NR¹¹R¹², aryl, heteroaryl, carbocyclyl, heterocyclyl,

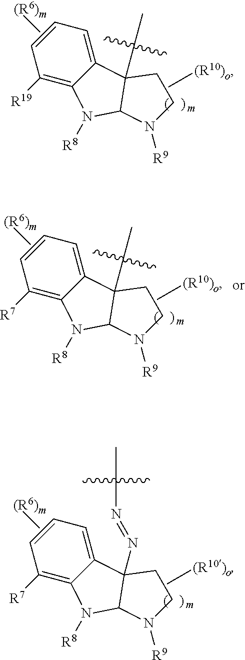

wherein
R⁷ and R^{7′} are each independently selected from H, —N₃, —N(R¹⁵)NH₂, —NHR¹⁵,

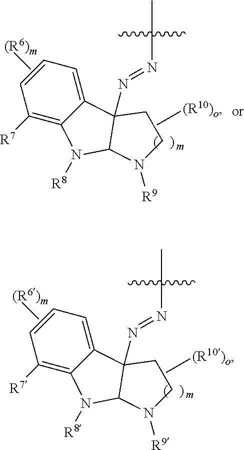

and wherein
R¹⁹ and R^{19′} are each independently selected from H, tertiary alkyl, alkenyl, aryl, heteroaryl, carbocyclyl, heterocyclyl,

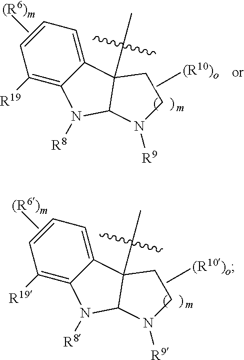

R⁸, R^{8′}, R⁹, and, R^{9′} are each independently selected from H, C₁-C₁₂ alkyl, C₂-C₁₂ alkenyl, C₂-C₁₂ alkynyl, —C(═O)R¹¹, —C(═O)OR¹¹. —C(═O)NR¹¹R¹², —SR¹¹, —S(═O)ₚR¹³, —S(═O)₂NR′R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

In another embodiment, the present disclosure provides a method of preparing the compound of Formula (II) by reacting compounds of Formula (III) and compounds of Formula (IV), wherein the compound of Formula (IV) is:

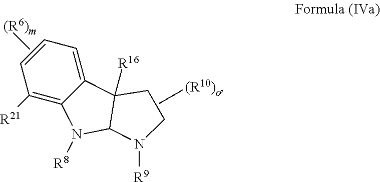

wherein R¹⁶ is I, Br, Cl, —OH, —OSO₂CF₃, —OS(O)₂R¹³, —OP(═O)R¹³R¹⁴, —OC(═NR¹¹)R¹², —OC(═NR¹¹)CCl₃, —OR¹¹, or —N₂⁺X⁻, wherein X⁻ is halogen;
R²¹ is H, halogen, alkyl, alkenyl, alkynyl, aryl, heteroaryl, carbocyclyl, heterocyclyl, —N₃,

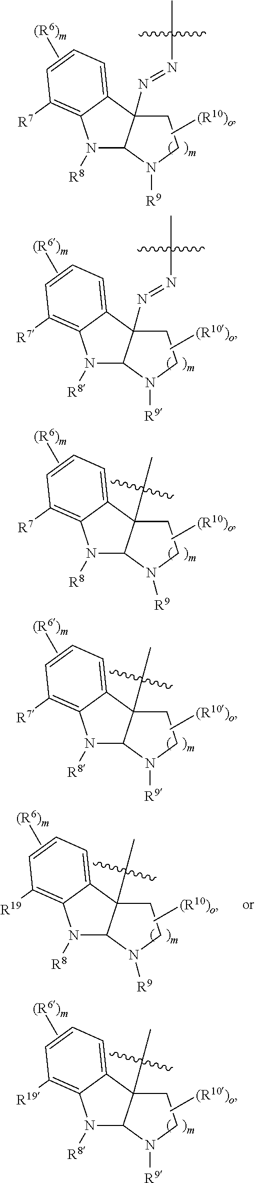

wherein R⁷ and R^{7′} are each independently selected from H, —N₃, —N(R¹⁵)NH₂, —NHR⁵,

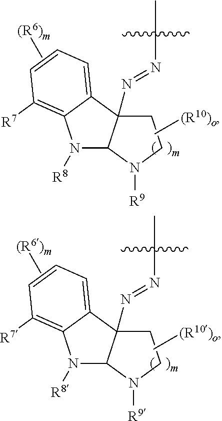

and
wherein R¹⁹ and R^{19′} are each independently selected from H, tertiary alkyl, alkenyl, aryl, heteroaryl, carbocyclyl, heterocyclyl,

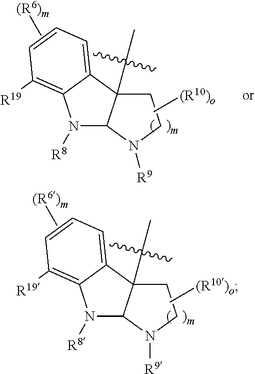

R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R^{8′}, R⁹, and, R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

In another embodiment, the present disclosure provides a method of preparing the compound of Formula (II) from the compound of Formula (IIIc) and the compound of Formula (IVa):

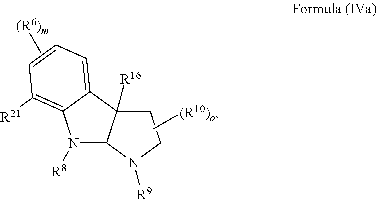

wherein R¹⁶ is I, Br, Cl, —OH, —OSO₂CF₃, —OS(O)₂R¹³, —OP(═O)R¹³R¹⁴, —OC(═NR¹¹)R¹², —OC(═NR¹¹)CCl₃, —OR¹¹, or —N₂⁺X⁻, wherein X⁻ is halogen;
R²¹ is H, halogen, alkyl, alkenyl, alkynyl, aryl, heteroaryl, carbocyclyl, heterocyclyl, —N₃,

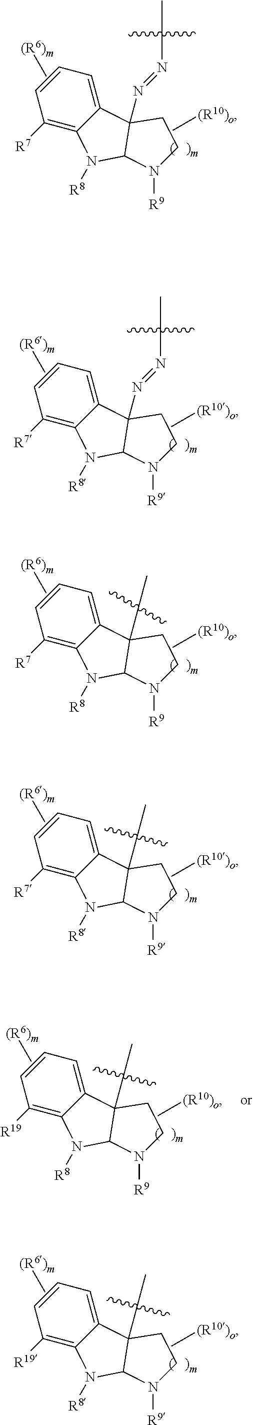

wherein
R⁷ and R^{7′} are each independently selected from H, —N₃, —N(R¹⁵)NH₂, —NHR¹⁵,

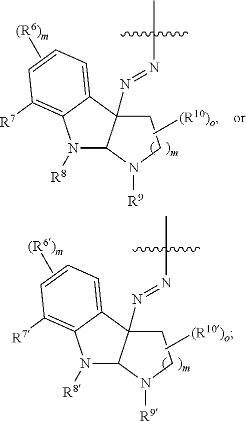

and wherein
R¹⁹ and R^{19′} are each independently selected from H, tertiary alkyl, alkenyl, aryl, heteroaryl, carbocyclyl, heterocyclyl,

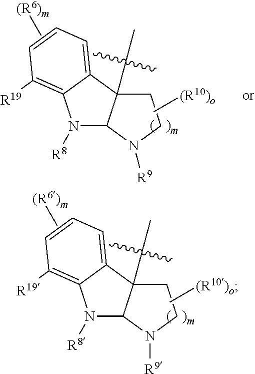

R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R^{8′}, R⁹, and, R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹; R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

In various embodiments, the present disclosure provides a method of preparing the compound of Formula (II) by the extrusion of sulfur dioxide from compounds of Formula (V):

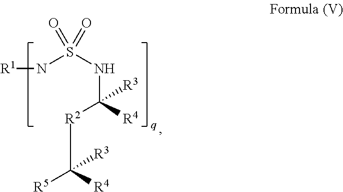

wherein R¹ is tertiary alkyl, alkenyl, aryl, heteroaryl, carbocyclyl, or heterocyclyl or at least one moiety of structure:

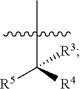

and

R², R³, R⁴, and R⁵ are each occurrence, each independently selected from alkyl, aryl, heteroaryl, carbocyclyl, or heterocyclic, wherein any two of R³, R⁴, and R⁵ taken together with the carbon atoms to which they are attached form a C₅₋₁₄ membered saturated, unsaturated, or aromatic carbocyclic ring or a C₅₋₁₄ membered saturated, unsaturated, or aromatic heterocyclic ring; and wherein any tertiary alkyl, aryl, heteroaryl, carbocyclyl, or heterocyclyl, C₅₋₁₄ membered saturated, unsaturated, or aromatic carbocyclic ring or a C₅₋₁₄ membered saturated, unsaturated, or aromatic heterocyclic ring can be further substituted with one or more halogen, alkyl, heteroaryl, carbocyclyl, heterocyclyl, C₃₋₁₄ membered saturated, unsaturated, or aromatic carbocyclic, or C₃₋₁₄ membered saturated, unsaturated, or aromatic heterocyclic rings; and q is an integer of from 0-8. In some embodiments, q is 1, 2, 3, 4, 5, 6, 7, or 8. In other embodiments, q is an integer from 0 to 1, 0 to 2, 0 to 3, 0 to 4, 0 to 5, 0 to 6, or 0 to 7.

In some embodiments the extrusion of sulfur dioxide is carried out in the presence of an oxidizing reagent. In some embodiments, the oxidizing reagent is N-chlorosuccinimide, N-chloro-N-methyl benzamide, 1,3-dichloro-5,5-dimethylhydantoin, trichloroisocyanuric acid, N-bromosuccinimide, 1,3-dichloro-5,5-dimethylhydantoin, iodosobenzene, PhI(OAc)₂, or PhI(OCOCF₃)₂. In one specific embodiment, the oxidizing reagent used to carry out the extrusion of sulfur dioxide is 1,3-dichloro-5,5-dimethylhydantoin.

In another embodiment, the method as disclosed herein is useful for the preparation of the compound of Formula (II), wherein the compound of Formula (V) is:

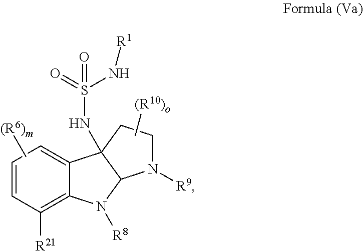

wherein
R¹ is tertiary alkyl, alkenyl, aryl, heteroaryl, carbocyclyl, heterocyclyl,

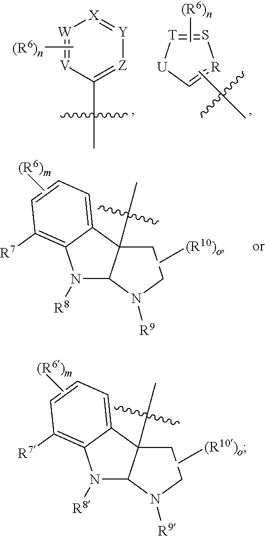

V, W, X, Y, and Z are each independently selected from —CH or N;
R, S, and T are each independently selected from —CH or N;

### U is O, S, or NR¹¹;

R²¹ is H, halogen, alkyl, alkenyl, alkynyl, aryl, heteroaryl, carbocyclyl, heterocyclyl, —N₃,

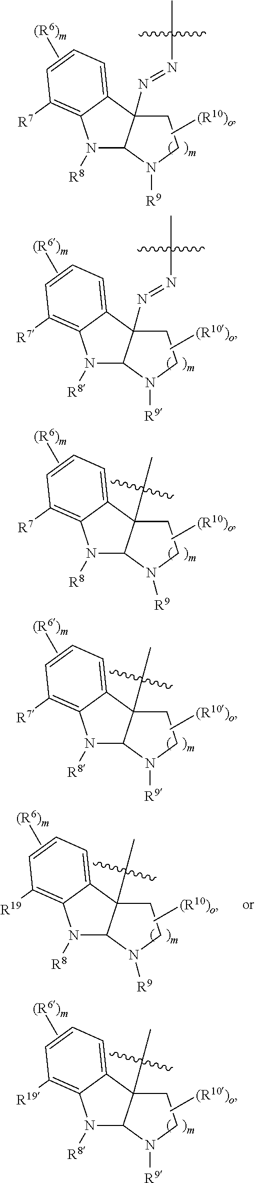

wherein
R⁷ and R^{7′} are each independently selected from H, —N₃, —N(R¹⁵)NH₂, —NHR⁵,

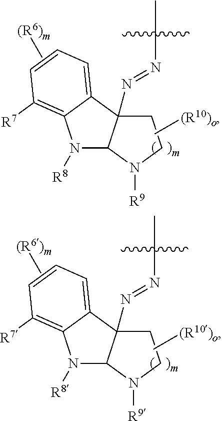

and wherein
R¹⁹ and R^{19′} are each independently selected from H, tertiary alkyl, alkenyl, aryl, heteroaryl, carbocyclyl, heterocyclyl,

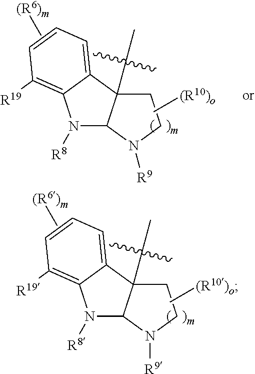

R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R^{8′}, R⁹, and, R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR², —C(═O)CF₃, —C(═O)OR²⁰, —P(═O)R¹³R¹⁴, or, —P(═O)NR¹¹R¹²;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

In other various embodiments, the present disclosure provides a method of preparing compounds of Formula (V) according to the following steps:

a. reacting a compound of Formula (VI) and a compound of Formula (IV) to give a compound of Formula (VII):

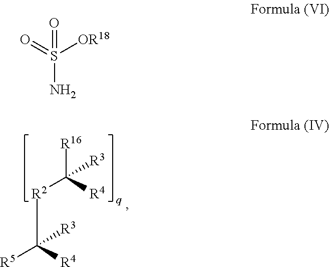

wherein
R¹⁶ is I, Br, Cl, —OH, —OSO₂CF₃, —OS(O)₂R¹³, —OP(═O)R¹³R¹⁴, —OC(═NR¹¹)R¹², —OC(═NR¹¹)CCl₃, —OR¹¹, or —N₂⁺X⁻, wherein X⁻ is halogen; and
R¹⁸ is aryl, or heteroaryl; and
R², R³, R⁴, and R⁵ are defined as above for Formula (I).
b. reacting a compound of Formula (VII) and a compound of Formula (VIII) to provide the compound of Formula (VI):

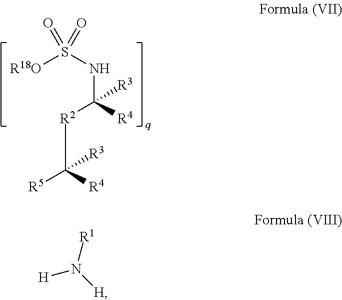

R¹ is tertiary alkyl, alkenyl, aryl, heteroaryl, carbocyclyl, or heterocyclyl.

In another embodiment, the method as disclosed herein is useful for the preparation of the compound of Formula (V) from the compound of Formula (VIII) and the compound of Formula (VII), wherein the compound of Formula (VII) is:

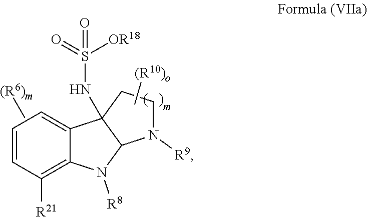

wherein R²¹ is H, halogen, alkyl, alkenyl, alkynyl, aryl, heteroaryl, carbocyclyl, heterocyclyl, —N₃,

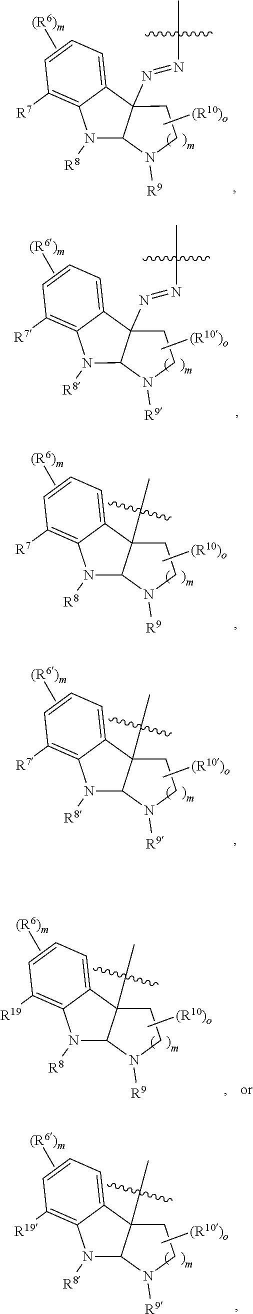

wherein
R⁷ and R^{7′} are each independently selected from H, —N₃, —N(R¹⁵)NH₂, —NHR¹⁵,

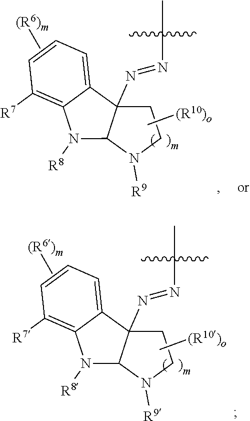

and
wherein R¹⁹ and R^{19′} are each independently selected from H, tertiary alkyl, alkenyl, aryl, heteroaryl, carbocyclyl, heterocyclyl,

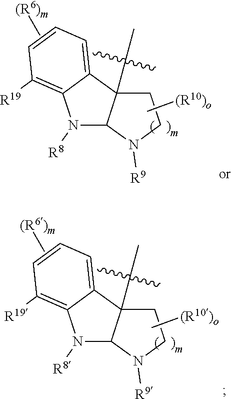

R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R^{8′}, R⁹, and, R^{9′} are each independently selected from H, C₁-C₁₂ alkyl, C₂-C₁₂ alkenyl, C₂-C₁₂ alkynyl, —C(═O)R¹¹, —C(═O)OR¹¹. —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)R¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;

### R¹⁸ is

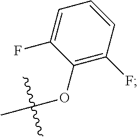 [SMILES: *Oc1c(F)cccc1F]

R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

In other various embodiments, the method as disclosed herein is useful for preparing the compound of Formula (V) from the compound of Formula (VII), and the compound of Formula (VIII), wherein the compound Formula (VIII) is:

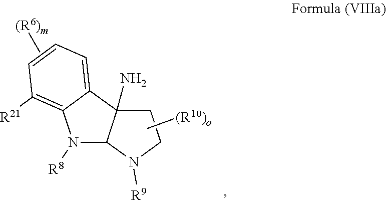

wherein R²¹ is H, halogen, alkyl, alkenyl, alkynyl, aryl, heteroaryl, carbocyclyl, heterocyclyl, —N₃,

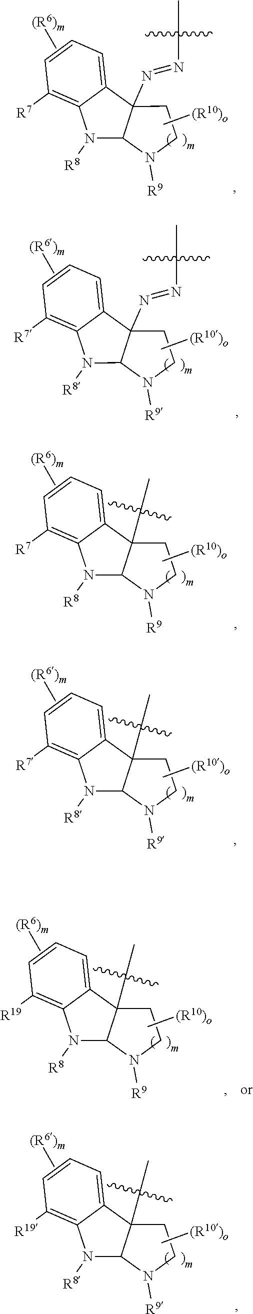

wherein R⁷ and R^{7′} are each independently selected from H, —N₃, —N(R¹⁵)NH₂, —NHR¹⁵,

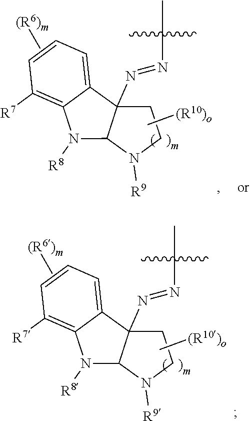

and wherein
R¹⁹ and R^{19′} are each independently selected from H, tertiary alkyl, alkenyl, aryl, heteroaryl, carbocyclyl, heterocyclyl,

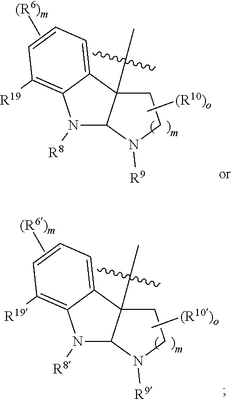

R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R^{8′}, R⁹, and, R^{9′} are each independently selected from H, C₁-C₁₂ alkyl, C₂-C₁₂ alkenyl, C₂-C₁₂ alkynyl, —C(═O)R¹¹, —C(═O)OR¹¹. —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)R¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

In other various embodiments, the method as disclosed herein is useful for preparing the compound of Formula (V) from the compound of Formula (VIIa), and the compound of Formula (VIIIa), as described above.

In other various embodiments, the method as described herein is useful for the preparation of the following compounds of Formula (I):

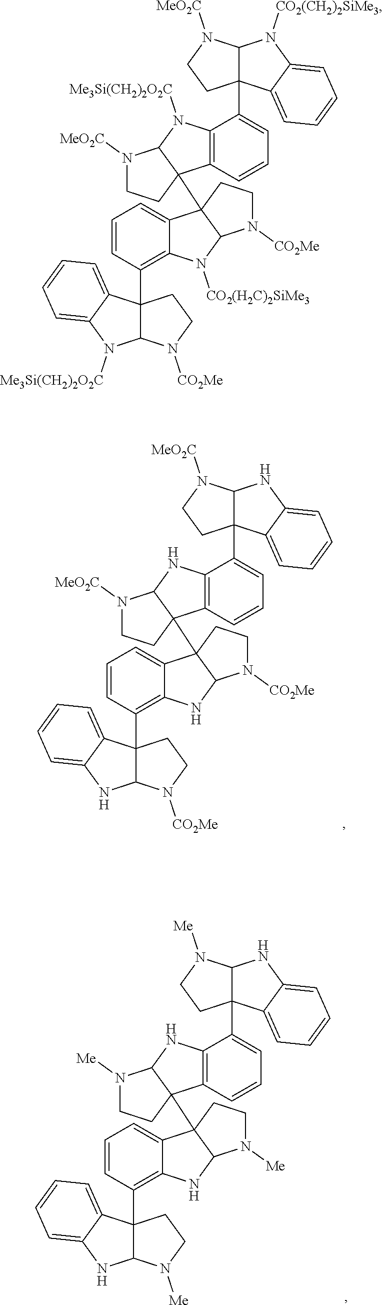

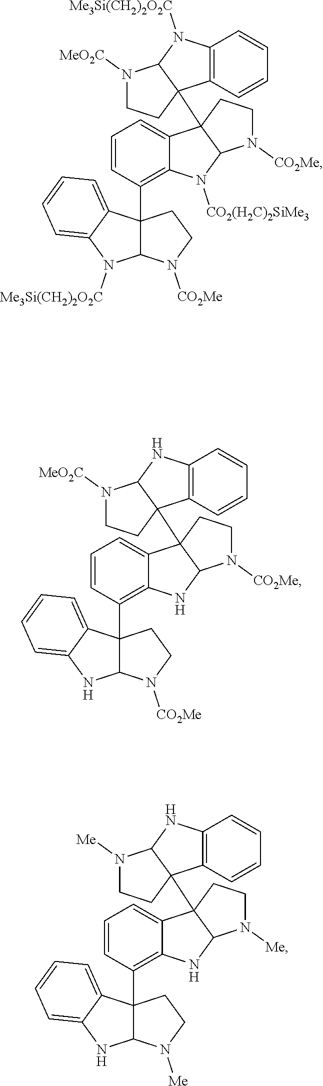

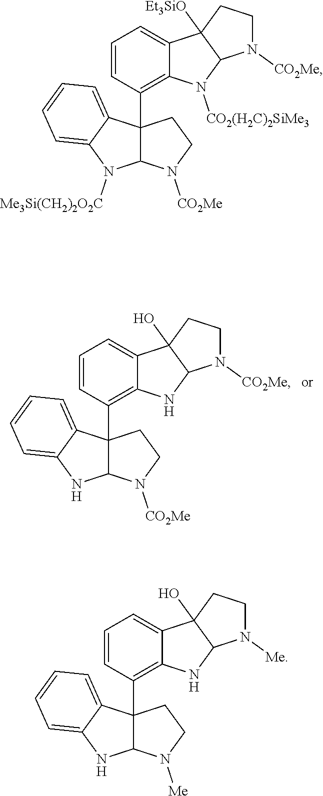

The union of tertiary electrophiles with N1,N1-disubstituted hydrazines containing electron withdrawing groups (EWG) provides diazene intermediates that are further transformed to afford the quaternary stereocenters disclosed herein. It is to be understood that although reference is made to exemplary EWGs, the disclosure is not limited to the EWGs discussed, and additional EWGs are within the scope of the disclosure. The disclosure provides examples of arenes where it is understood that the disclosed syntheses are general in nature, and thus applicable to additional compounds, including but not limited to a variety of heteroarenes, heterocycles, aryl hydrazine components, diazene derivatives, and ultimately the quaternary carbon-containing products that result.

The disclosure also provides a modular synthesis, with examples of polycyclotryptamines and oligocyclotryptamines provided. Specifically, diazene-directed modular synthesis of (−)-quadrigemine C, (−)-hodgkinsine, and (−)-hodgkinsine B is described, and it is to be understood that the methods and approaches discussed herein can be generalized and applied to the synthesis of a variety of compounds, such as those represented by Formula (I). The disclosed methods and processes of diazene-directed modular synthesis provide for preparing a variety of compounds where the stereochemistry of each tricycle is secured before fragment coupling. It is to be understood that variations in the benzene substructure, amine alkyl groups, relative stereochemistry of subunits, the number of subunits, and the number of subunits on each tail (moving away from the Csp3-Csp3 center), are within the scope of the disclosure.

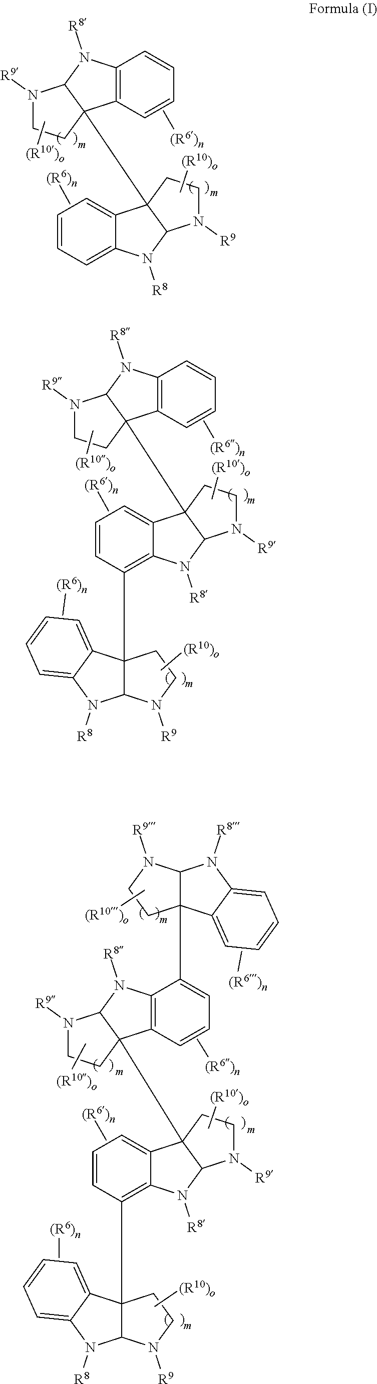

A summary of the advancements facilitating the implementation of new and inventive syntheses, including syntheses of oligocyclotryptamines and related compounds, is provided below. The methods disclosed describe the sole strategy available to access these compounds using a modular and iterative approach that secures the absolute and relative stereochemistry of each cyclotryptamine component. The advancements include:

(1) Development of a new class of hydrazide nucleophiles capable of generating diazenes intermediates directly. This new class avoids complications with oxidation conditions normally needed for diazene synthesis that are incompatible with structures of oligocyclotryptamines. Our approach to diazene synthesis has revolutionized synthesis of diazenes intermediates, complex diazenes, and strategies for fragment assembly, and has numerous additional applications.

(2) Demonstration of the first example of C3-C7′ coupled dimeric cyclotryptamines through expulsion of dinitrogen and “fusion of two cyclotryptamine fragments”. This required the synthesis of diazene intermediate that were previously inaccessible and demonstrated their ability to lose dinitrogen and afford the desired C—C bond of interest.

(3) Development of strategies to carry out coupling of complex fragments and functionalization of sterically crowded substrates. This required development of strategies that rely on “spatial separation of functionalization chemistry from the sterically crowded positions in the substructures”.

(4) Illustration of the synthesis of dimeric, trimeric, and tetrameric oligocyclotryptamines with applications in many other members of the family that have otherwise been inaccessible through chemical synthesis.

(5) Demonstration that this new technology allows controlled modular synthesis of oligocyclotryptamines and derivatives of these and related alkaloids. The chemistry provides the only stereochemically unambiguous solution to a unique class of alkaloids with known biological activity (with application in neurochemistry, neurodegenerative diseases, use as anticancer agents, use as analgesics, etc.) with impact in areas such as Alzheimer's disease, cancer, addiction, and pain treatment. It has been established that the bioactivity of these compounds is directly proportional to the length of the oligomeric chain. Thus, the controlled and modular synthesis of oligomers is a significant advancement with a wide range of applications.

(6) Clarification of the complex stereochemistry possessed by oligotryptamines and derivatives. Compounds such as these are typically very rare in nature and the small samples obtained from nature are plagued by stereochemical ambiguity and uncertainty due to the overlapping signals of the repeating units, hindered bond rotation leading to line broadening due to severe steric congestion, and instability of the compounds to heat and oxidation. Samples prepared by the disclosed methods are formed with predetermined and established stereochemistry in an iterative fashion, thus allowing ease of assignment of the final oligomeric product.

The methods described herein are exemplified with representative synthesis as follows. The demonstration begins with the establishment of an efficient and adaptable synthesis of key building blocks. For example, the following synthesis of versatile monomers is useful for application in the diazene directed modular synthesis of oligocyclotryptamines, as well as other differentially substituted derivatives.

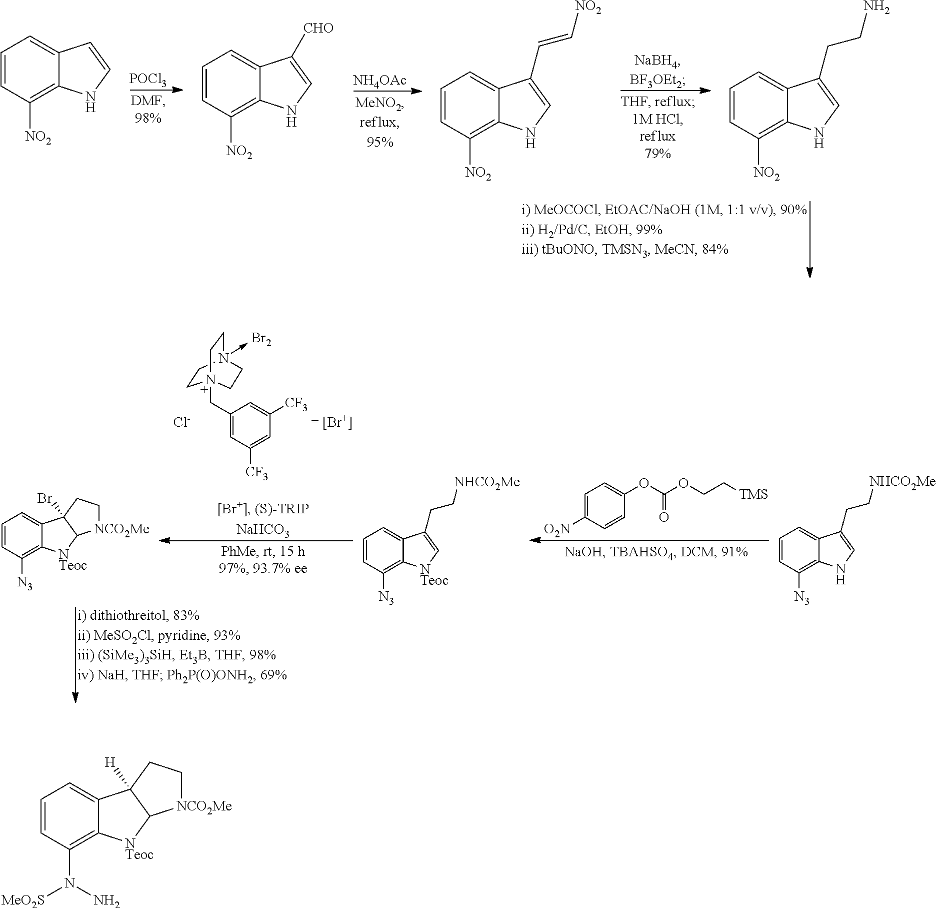

The cyclotryptamine building block provides the repeating tricyclic portion of oligocyclotryptamines, and is properly functionalized for further chemistry. Notably, it contains:
(i) our designed (1) N1-aryl N1-sulfonyl hydrazide portion that we have developed for direct access to diazene upon trapping of carbocations;
(ii) a specific indoline nitrogen protecting group (Teoc, trimethylsilylethoxycarbonyloxy) capable of “remote” deprotection at the final stage of synthesis in a crowded environment of oligocyclotryptamines;
(iii) azide as the precursor for the key sulfonyl hydrazide to allow introduction of the requisite nucleophilic functional group useful in the enantioselective synthesis.

The above timing and order of events can be critical to the success of the synthesis of this building block, according to some embodiments. Although the building block has been experimentally demonstrated to have utility in the synthesis of dimeric, trimeric, and tetrameric cyclotryptamines herein, it can be applied in numerous other applications and to numerous other syntheses.

In addition, the necessary C7-sulfonyl hydrazine can be established by an alternative synthesis starting from readily available materials. The C3a-bromocyclotryptamine derivative can be reduced in high yield to the corresponding C3a-H cyclotryptamine using (Me₃Si)₃SiH/Et₃B. Subsequent treatment with methanesulfonyl azide in the presence of an Ir-catalyst and silver salt promoters leads to a carbamate directed C7-sulfonamidation of the cyclotryptamine structure. The sulfonamide is then converted to the desired sulfonylhydrazine via direct amination of the sulfonamide. One of the benefits of this method is the opportunity to utilize starting materials unsubstituted at the C7 position.

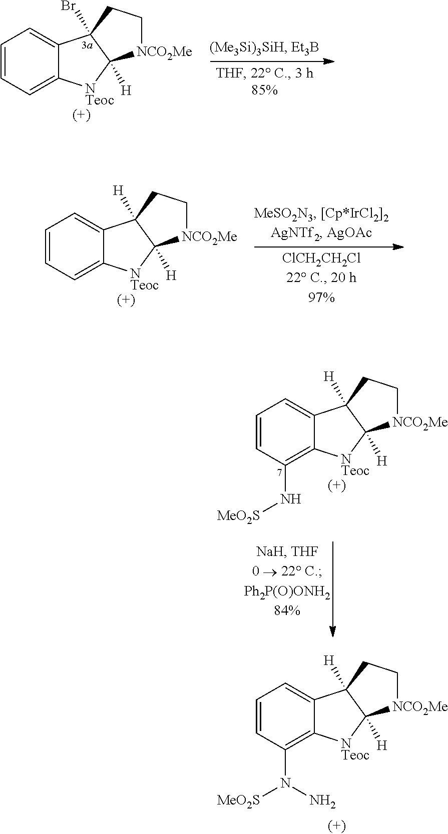

The building blocks described, as well as a range of other electron withdrawing group-substituted hydrazines, have demonstrated utility in the synthesis of diazene intermediates that serve as precursors to C—C bond formation. The following representative series show aspects of the development of the novel technology to access oligocyclotryptamines using a modular diazene-directed synthesis. In these cases, a Csp2-Csp3 bond is formed.

The Use of N1-Aryl N1-Boc Aryl Hydrazine:

A notable advance is the discovery of a hydrazine derivative that is compatible with conditions needed for electrophilic activation of the readily available cyclotryptamine-bromide starting material. The introduction of the electron-withdrawing group on the nitrogen of the hydrazine allows its exposure to silver (I) without undergoing undesired decompositions via redox chemistry. Another advance is that the necessary N—N bond of the projected diazene is already present when using a hydrazine precursor. This aspect of the disclosure has implications and applications for all other diazene related chemistry.

Successful Addition of Hydrazine Nucleophile:

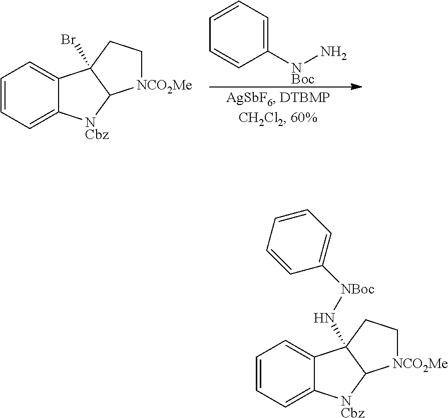

Use of Trifluoroacetylated Hydrazines as Nucleophiles:

The trifluoroacetyl is compatible with mild removal of the acetyl group and oxidation to the desired N-aryl N-cyclotryptaminyl diazene. This strategy allows the first synthesis of cyclotryptamine C3-aryldizene using nucleophilic hydrazine derivatives, retaining the preexisting C3-stereochemistry in the product.

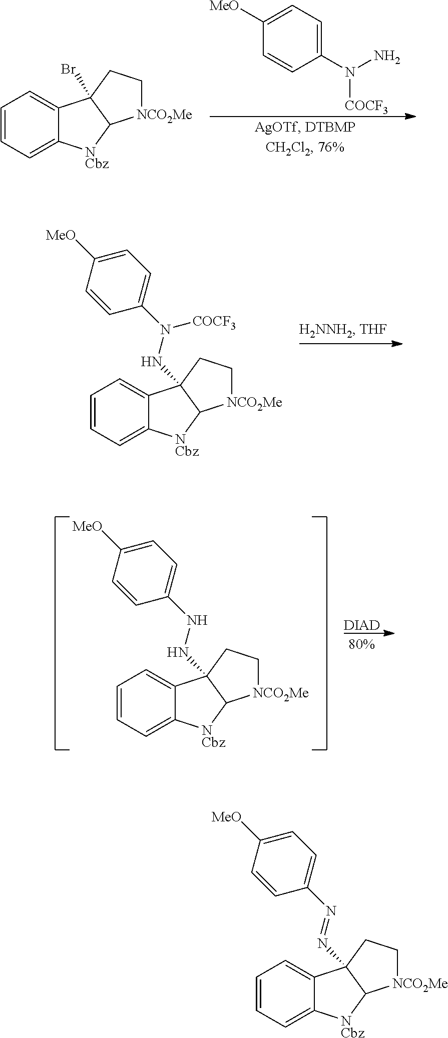

Use of Methanesulfonylated Hydrazines as Nucleophiles:

In addition to the efficient preparation of the C7-sulfonylhydrazine compounds disclosed above, the use of a methanesulfonyl electron-withdrawing on the hydrazine nucleophile has the additional benefit of reducing the overall three step sequence in the previous example to a single step, according to some embodiments. This has revolutionized synthesis of these and other alkyl diazenes. This approach has applications in many other areas, including but not limited to synthesis of heteroaryl derivatives, which are currently being pursued using an alternative, less efficient strategies.

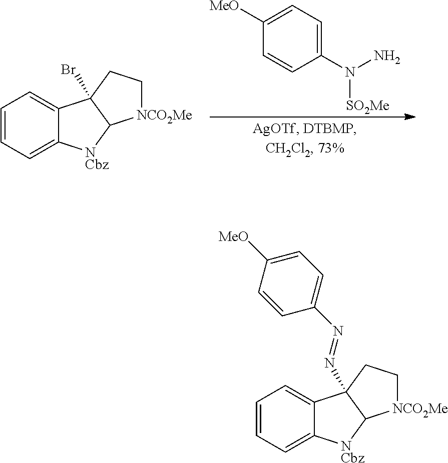

Successful Cyclotryptamine Diazene Synthesis:

The synthesis and use of such N-sulfonyl aryl hydrazine nucleophiles is applicable to challenging substrates and coupling, as shown below. The necessary cyclotryptamines hydrazines needed as repeating substructure of oligocyclotryptamines can be prepared. The example shown represents a successful synthesis of a dimeric cyclotryptamine diazene product.

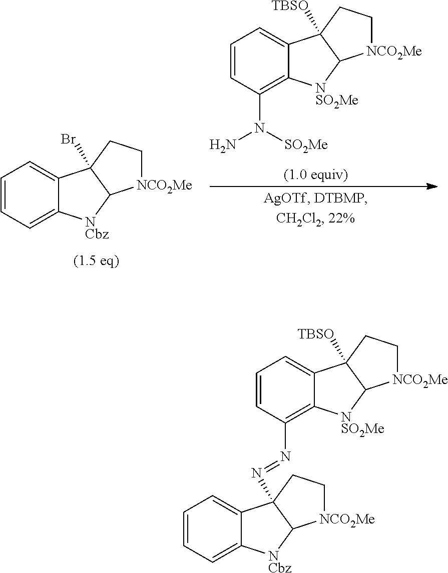

Use of Indoline Nitrogen Protecting Group Allowing Final Stage Removal:

Access to dimeric diazenes revealed the significant challenges in both introduction and removal of the internal indoline protective group once attachments are introduced at the C7-position. While it was discovered that the use of Teoc (trimethylsilylethoxycarbonyloxy) is useful since the deprotection is initiated “remote” (nucleophilic addition at the silicon of trimethylsilyl group) from the congested linkage area, the tight steric pressure at the juncture greatly complicates carrying out chemistry close to the linkage. Even a labile o-nitrobenzenesulfonyl group, typically an easily removable group, once introduced at the internal indoline nitrogen is not subject to deprotection under a variety of conditions due to severe steric pressures. In cases where a free internal nitrogen is deemed desirable, Teoc has proven to be a useful protecting group. However, it is the function, and not the particular group that is the relevant element. Other groups possessing the similar feature of carrying out the chemistry away from the steric congestion can alternatively be used. Furthermore, silyl group variants are also possible. In one embodiment, use of a triisopropylsilyl version instead of trimethylsilyl can provide a similar result, although Teoc is typically preferred.

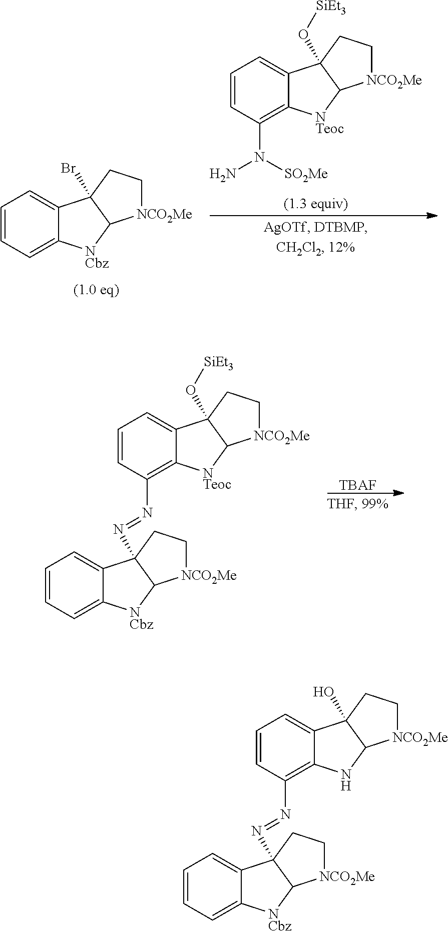

The efficiency of Teoc deprotection in the case above can be compared with the challenges shown in the case where nosylate is used to protect the internal indoline nitrogen.

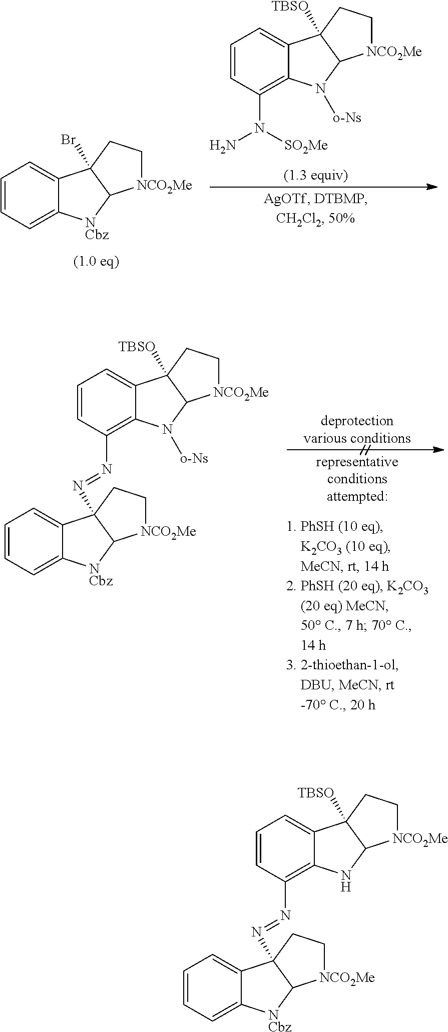

Successful Modular Synthesis of C3-C7 Dimeric Cyclotryptamine Synthesis:

The synthesis of the diazene linked dimer and its conversion to the C—C linked dimer is applicable on a range of substrates. Above we show the repeating unit to highlight the prospects of a controlled chain growth in accessing oligocyclotryptamines. With synthesis of the first diazene linked dimeric cyclotryptamine we proceeded to accomplish the first fusion of the two fragments as shown above constituting the first synthesis of a C3-C7 dimeric cyclotryptamine via this novel strategy.

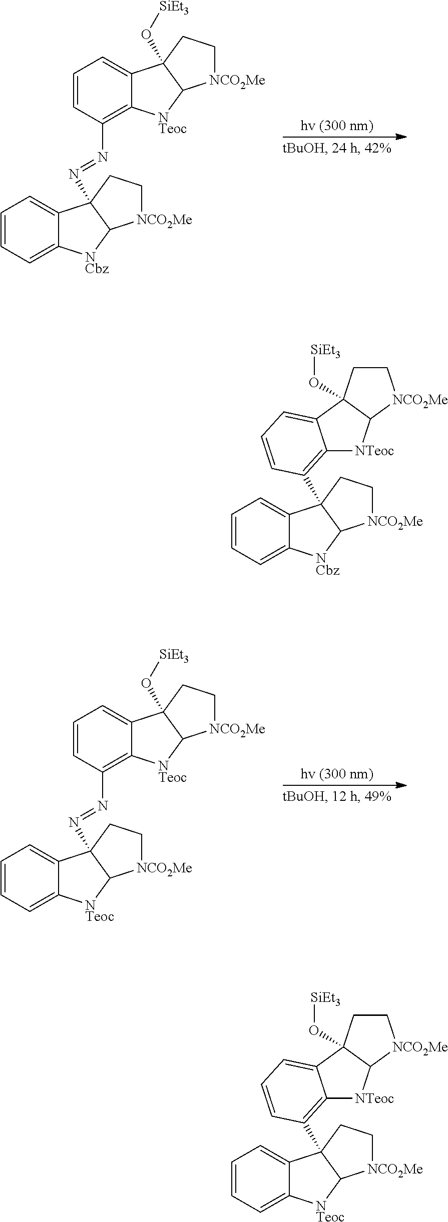

Strategy for “Chain Extension” Via CH-Amination of Dimeric Substrates:

As an alternative to our electrophilic activation at C3 to give coupling products, we developed a strategy based on CH-amination chemistry that alternatively affords the formation of a Csp3-Csp3 bond. This approach is applicable starting from either stereochemical combination (R or S) as shown in the following two examples. Furthermore, the CH-amination approach is also applicable to substrates possessing a pre-existing diazene moiety, or one where the C—C linkage is already established.

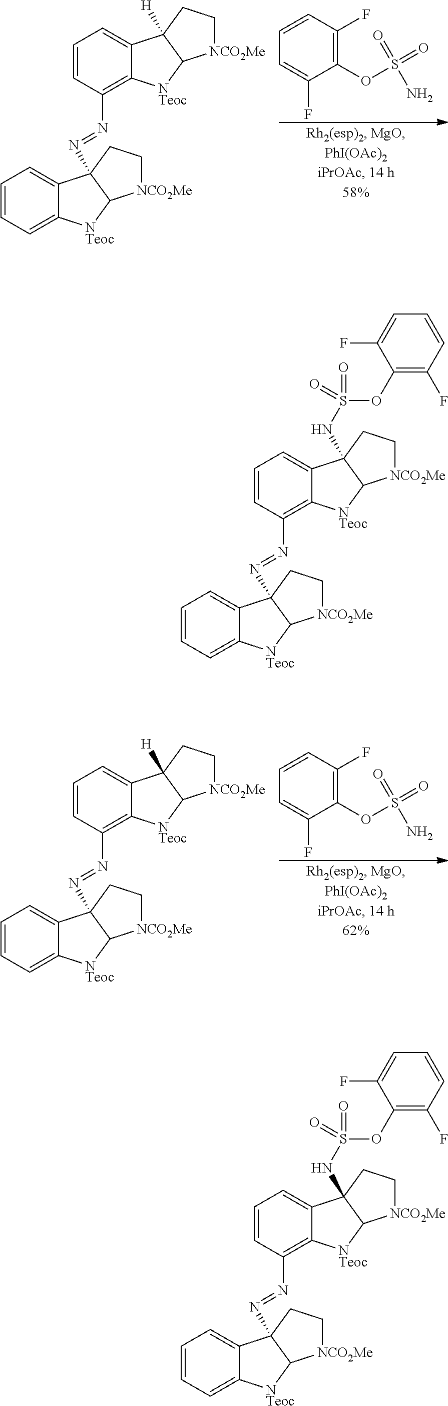

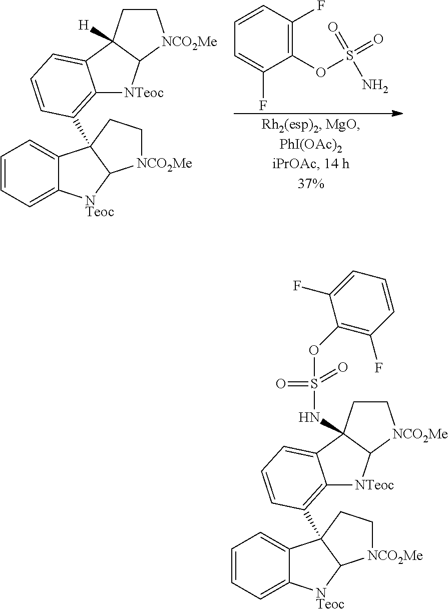

The added versatility of the CH-amination method increases the range of possible target molecules, as it too is demonstrated to be operable in complex molecular environments, as exemplified in the successful synthesis of (−)-quadrigemine C. Application of both the Csp2-Csp3 and Csp3-Csp3 bond forming reactions to the synthesis of trimeric and tetrameric cyclotryptamines serves to illustrate the robust nature of the chemistry developed.

The retrosynthetic analysis of (−)-quadrigemine C, a tetrameric cyclotryptamine:

A modular synthesis employing simple cyclotryptamines as starting material that are fused together via our diazene chemistry is provided. This requires unprecedented multiple diazene fragmentations between from Csp2-Csp3 centers and Csp3-Csp3 centers to assemble the structure. No prior example in complex synthesis exists using such complex diazenes or the iterative use of mono- or poly-diazene intermediates in assembly of multiple fragments. Here four pieces are coupled by loss of three dinitrogen molecules as highlighted by the retrosynthesis below.

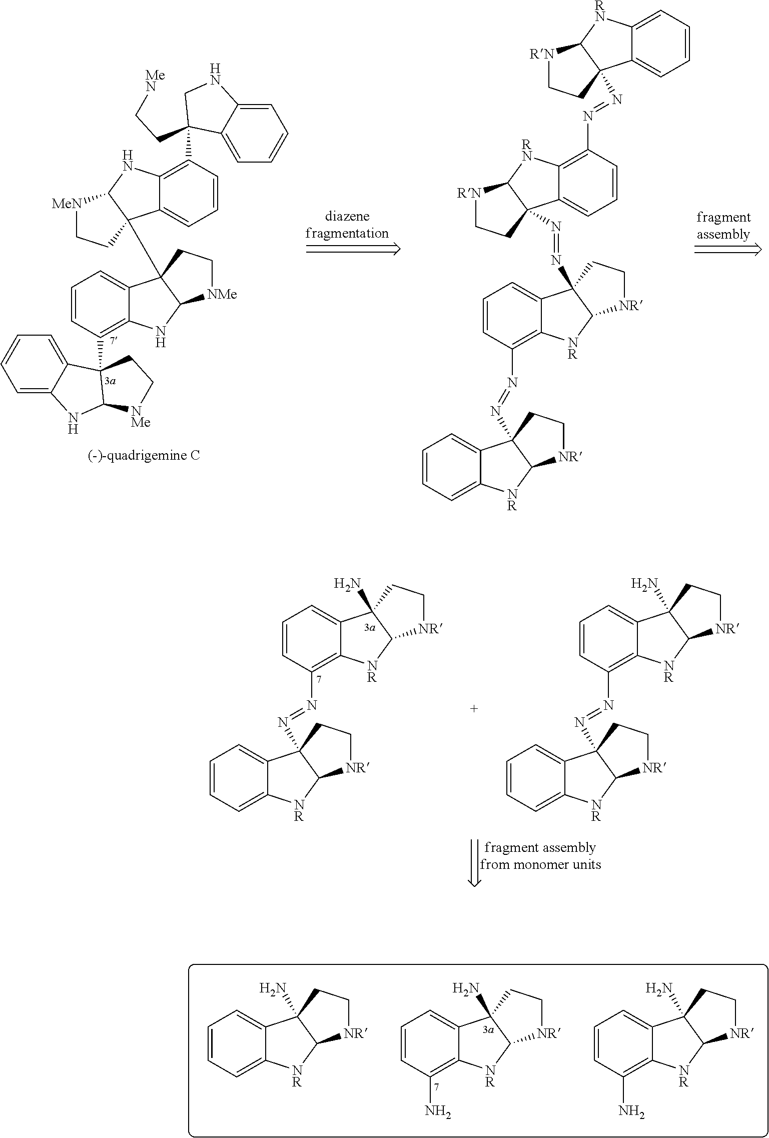

Iterative and Modular Cyclotryptamine-Chain Extension:

The illustrative example below shows how the disclosed modular strategy for the synthesis of (−)-quadrigemine C is begun. Highly efficient sulfonyl hydrazine coupling with a bromocyclotryptamine promoted by AgOTf and base gives rise to the diazene linked dimer in 56%. The building blocks are constructed from either C3-H halogenation, from C3-OH derivatives, or from C3-H amination of the functionalized cyclotryptamines as disclosed above, and form the foundation for some embodiments of our modular and iterative synthesis of oligocyclotryptamines.

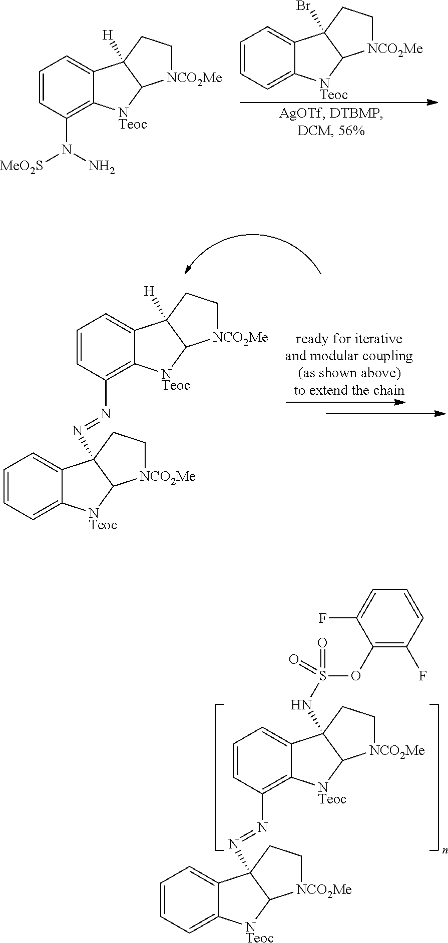

Successful Synthesis of the Proposed Precursor to (−)-Quadrigemine C:

Once the first diazene moiety is installed from Csp2-Csp3 coupling, the CH-amination method is initiated. Installation of the 2-6-difluorosulfamate occurs via CH-activation of the precursor to produce the electrophile ready for addition of a —NH₂-containing dimeric cyclotryptamine. DMAP promotes nucleophilic addition to obtain the tetrameric sulfamide product possessing Teoc groups at the internal indoline nitrogen positions, which undergoes extrusion of sulfur dioxide upon treatment with 1,3-dichloro-5,5-dimethylhydantoin (or other electrophilic chlorinating reagent) to provide the key diazene-containing tetrameric intermediate. Up to this point, the coupling of highly complex fragments where four cyclotryptamines are united via three diazene linkers has been surprisingly demonstrated. All components have predetermined absolute and relative stereochemistry, setting the stage from C—C bond formation to complete the framework of the natural product scaffold.

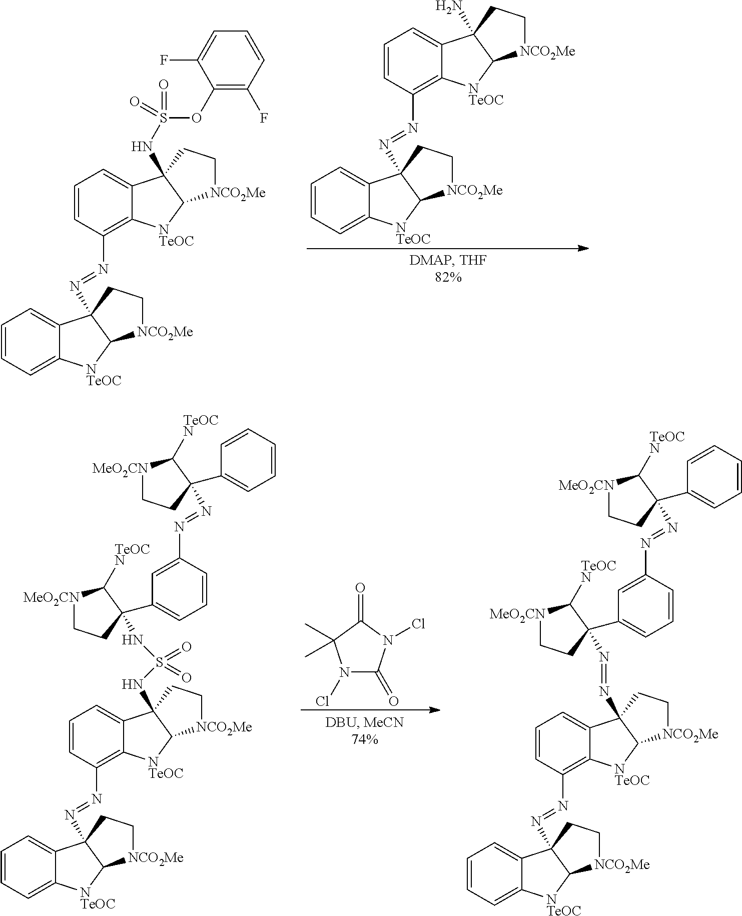

Successful Completion of the (−)-Quadrigemine C Synthesis Using the Modular Assembly Approach:

Photoexpulsion of the diazene linkers occurs in the next stage in stepwise fashion. As shown, the first irradiation at a higher wavelength (i.e., 380 nm) leads to expulsion of dinitrogen and formation of the Csp3-Csp3 bond uniting the C3 positions of the two internal cyclotryptamines. The second irradiation at a lower wavelength (i.e., 300 nm) results in formation of the two Csp2-Csp3 bonds between the external and internal cyclotryptamines to establish the requisite connectivity. Deprotection of TeOC and reduction then provides (−)-quadrigemine C.

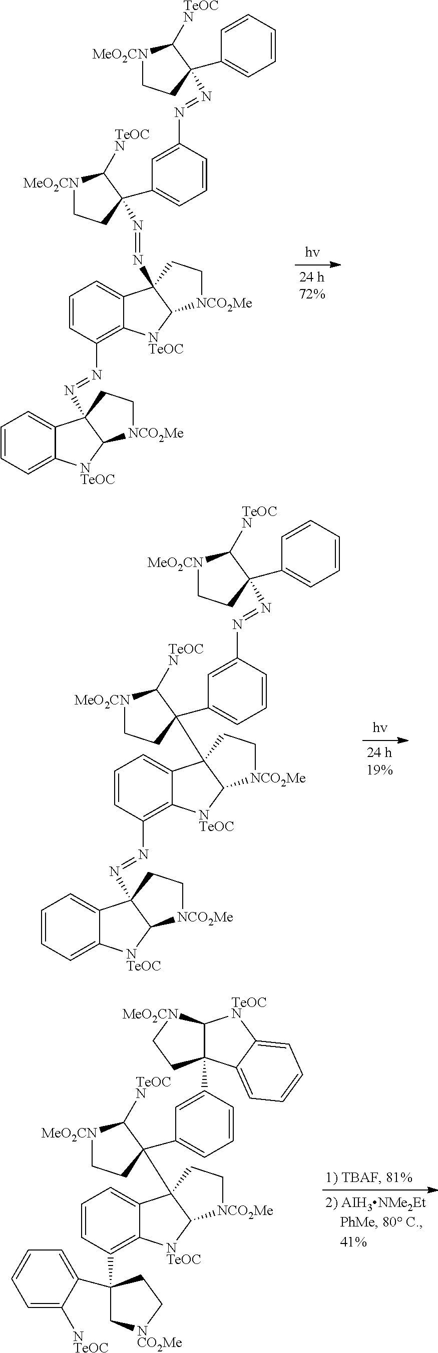

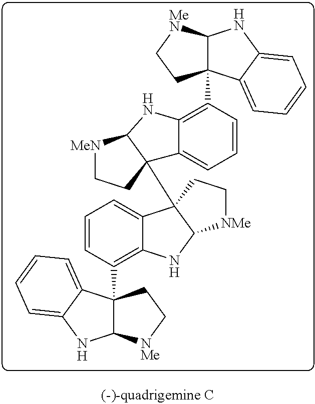 [SMILES: CN1CC[C@]2(c3cccc4c3N[C@@H]3N(C)CC[C@]43[C@@]34CCN(C)[C@@H]3Nc3c([C@]56CCN(C)[C@H]5Nc5ccccc56)cccc34)c3ccccc3N[C@H]12]

Of note in the above synthesis is that a complete control of stereochemistry and regioselectivity in fragment unions has been established in the total synthesis of (−)-quadrigemine C. Generation of four quaternary centers—very challenging structural features—is notable. The ability to distinguish between Csp3-Csp3 vs. Csp3-Csp2 linked diazenes in the fragmentation is also is a substantial achievement. In some embodiments, conditions can be specified to reduce the photoexpulsion of three molecules of dinitrogen from the starting material to a single step.

Successful Application to Synthesis (−)-Hodgkinsine and (−)-Hodgkinsine B Using the Modular Assembly Approach:

Similar to the tetrameric case above, complete control of stereochemistry and regioselectivity in fragment unions is achieved in the synthesis (−)-hodgkinsine and (−)-hodgkinsine B. For both natural products, an azide compound serves as a precursor to the electron withdrawing group-containing hydrazine, although methods are disclosed for alternative preparations of this key building block. Formation of the Csp2-Csp3 diazene occurs via silver (I) promoted electrophilic activation of a C3-bromide, followed by hydrazine addition. Next, the CH-amination reaction is carried out to install the sulfamate moiety. Nucleophilic addition of a C3-aminocyclotryptamine derived from the requisite C3-bromide intermediate, followed by subsequent SO₂ extrusion after treatment with an electrophilic chlorinating reagent affords the bis-diazene compound. Under irradiation conditions, dinitrogen is expelled and (−)-hodgkinsine and (−)-hodgkinsine B are each obtained after Teoc-deprotection and reduction of the methyl carbamate.

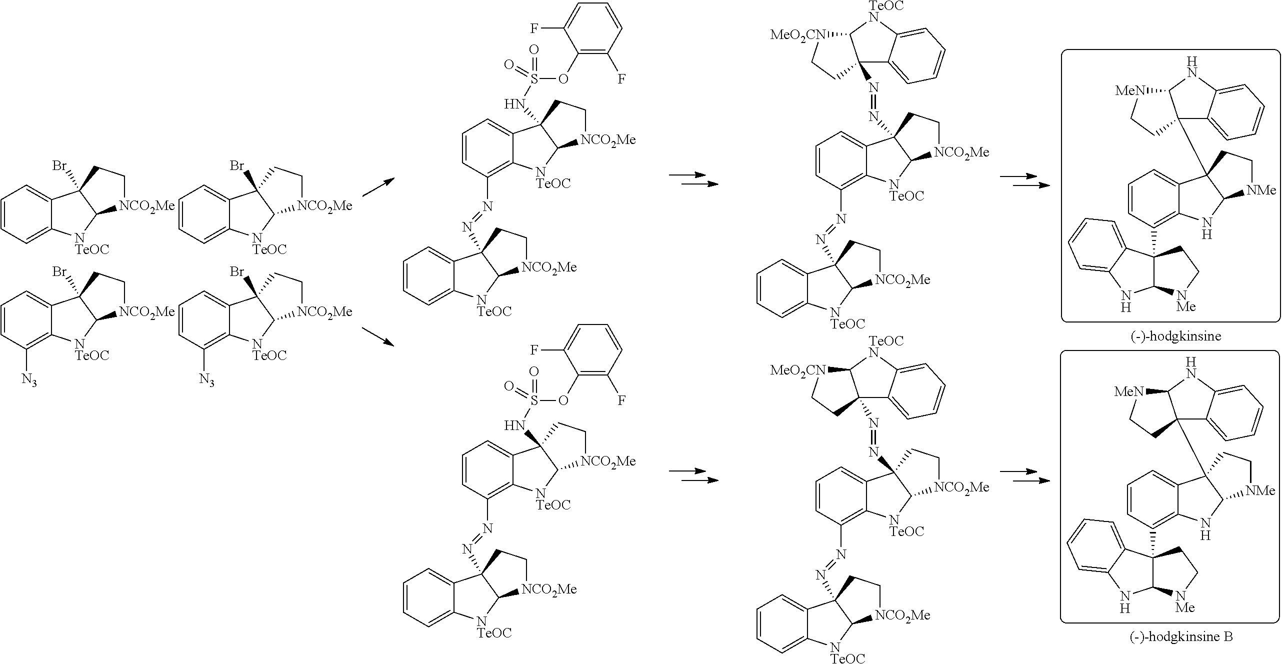

In summary, this technology and chemistry has application to many other areas beyond the examples provided, including but not limited to other arene hydrazines beyond those discussed herein. For ease of understanding, simple arenes and very complex arenes such as indolines are provided herein. However, many additional other compounds including many other heteroarenes (pyridines, pyrimidines, pyrroles, oxazoles, thiazoles, diazoles, etc.) can use a similar strategy for synthesis of the diazene intermediate and the desired product, and are within the scope of this disclosure.

The disclosed methods have application in a variety of fields, including pharmaceutical sciences, medicinal chemistry, and process chemistry, among others, as there are presently no other options for such regiospecific coupling. Transition metal chemistry does not work with such sterically crowded carbon centers for coupling, and other methods of coupling heteroarenes are plagued by competing regioisomer formation.

Although discussed with particular examples and compounds herein, the disclosure applies to many other electrophile classes. Here we have focused on tertiary electrophiles, and we know that secondary electrophiles will cause complications with the intermediate diazene (via tautomerization to a hydrazone). For example, some embodiments of the disclosure can utilize other cyclotryptamines, substituted on the arene-ring, the nitrogens, or the aminoethyl group. As another example, in some embodiments, other tertiary electrophiles can be utilized, including R, R′, and R⁴¹ as carbon or any derivative where one or two are heteroatoms.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

In addition, those of ordinary skill in the art recognize that some functional groups can be protected/deprotected using various protecting groups before a certain reaction takes place. Suitable conditions for protecting and/or deprotecting specific functional group, and the use of protecting groups are well-known in the art.

For example, various kinds of protecting groups are described in T. W. Greene and G. M. Wuts, Protecting Groups in Organic Synthesis, Second edition, Wiley, New York, 1991, and other references cited above.

All documents cited herein are herein incorporated by reference in their entirety for all purposes.

Generally, this disclosure includes a method for modular synthesis comprising:

(a) performing a coupling reaction between a tertiary electrophilic group (I) and a suitably protected N1,N1-disubstituted hydrazine (II) to provide a diazene (III);

(b) performing a photoexpulsion of a nitrogen molecule to obtain a coupled product with a quaternary stereocenter; and

(c) deprotecting the coupled product to obtain a dimeric cyclotryptamine (IV) wherein the general reaction scheme is as follows:

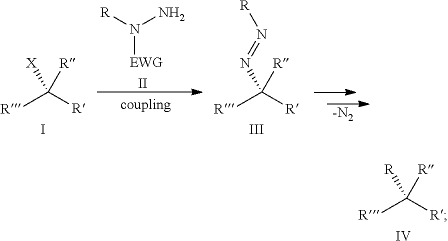

and

The method includes stereoselective synthesis strategies, and can further comprise additional steps/elements, including chain extension. As discussed herein, iterative and modular cyclotryptamine-chain extension is one such representative process:

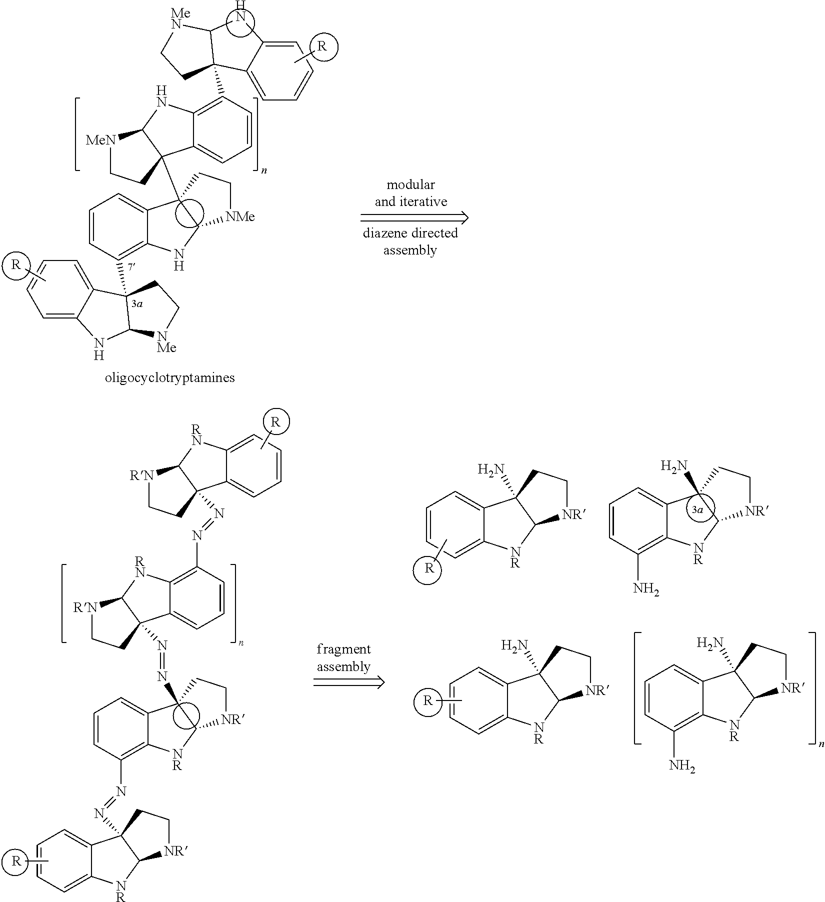

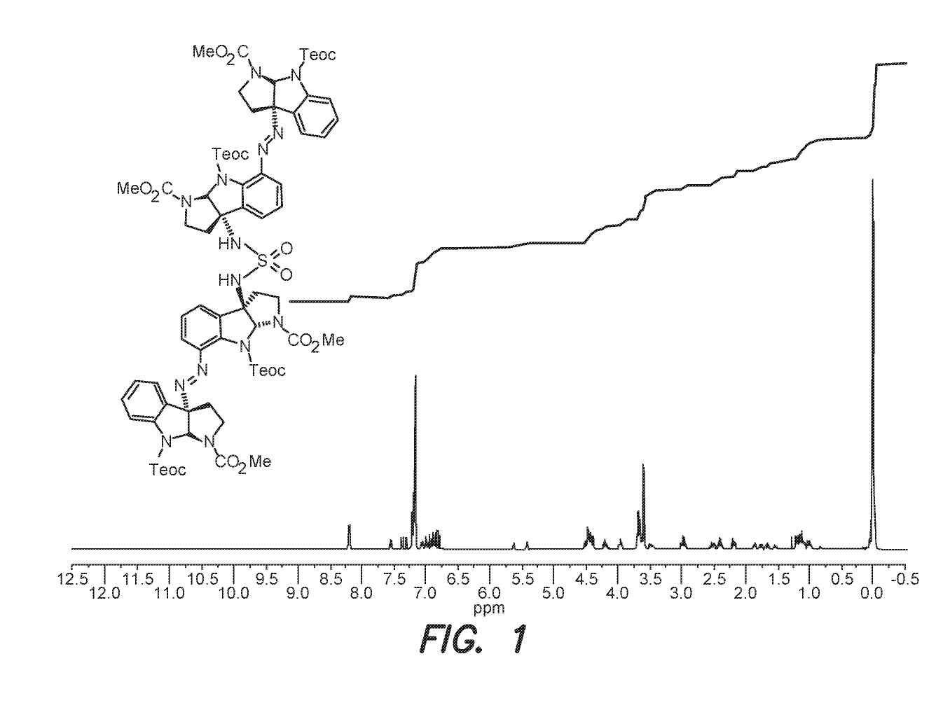

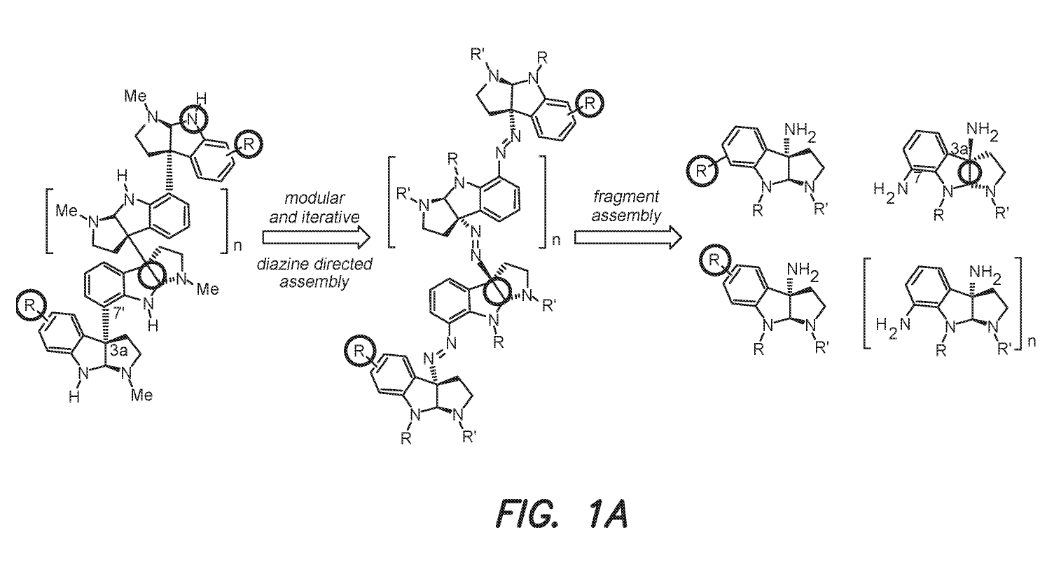

In some embodiments, as illustrated in FIG. 1A, application of this synthesis and related syntheses of the disclosure provide reliable and practical access to not only rare natural products but also various designed derivatives. Variations can be introduced elegantly to the monomers, and will include substitution on the indoline component (red and blue), late stage amine substitution (green), absolute and relative stereochemistry (purple), number of copies of any monomer (yellow).

Using the methods described herein, various stereogenic quaternary carbon-containing compounds, including oligocyclotryptamines, can be prepared from the appropriate starting materials and intermediates, as shown below in the following representative schemes.

### Example 1. Synthesis of (−)-quadrigemine C

### Sulfamide Formation

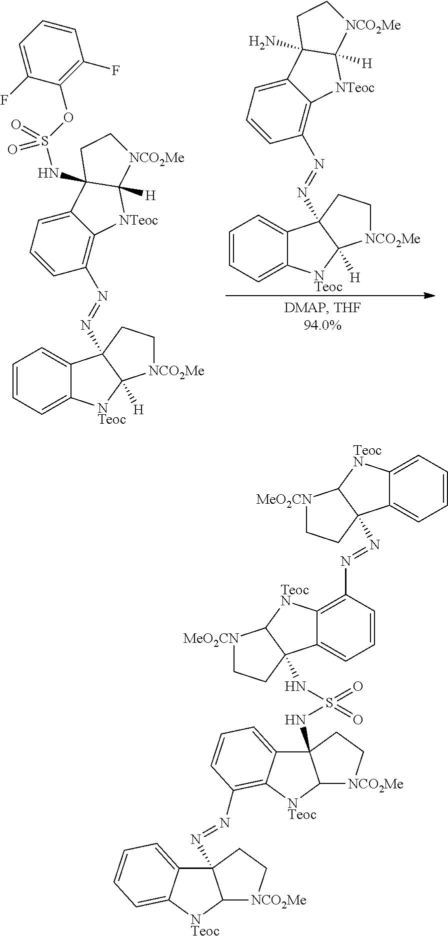

A sample of 4-(dimethylamino)pyridine (137 mg, 1.12 mmol, 2.20 equiv) was added to a solution of cyclotryptamine diazene sulfamate ester (490 mg, 511 μmol, 1 equiv) and cyclotryptamine diazene amine (430 mg, 562 μmol, 1.10 equiv) in tetrahydrofuran (5.10 mL) at 22° C. After 7 h, the bright yellow solution was concentrated under reduced pressure. The resulting residue was purified by flash column chromatography on silica gel (eluent: 30%→75% ethyl acetate in hexanes) to afford cyclotryptamine tetramer (766 mg, 94.0%) as a bright yellow amorphous gum.

As a result of the slow conformational equilibration at ambient temperature, NMR spectra were collected at elevated temperature. Structural assignments were made using additional information from gCOSY, HSQC, and HMBC experiments also collected at elevated temperature.

¹H NMR (400 MHz, C₆D₆, 70° C.): δ 8.19 (d, J=8.1 Hz, 2H), 7.53 (d, J=7.4 Hz, 1H), 7.36 (d, J=8.0 Hz, 1H), 7.30 (d, J=8.0 Hz, 1H), 7.22-7.14 (m, 6H), 7.05 (d, J=7.3 Hz, 1H), 6.99 (td, J=7.5, 0.7 Hz, 1H), 6.93 (t, J=7.8 Hz, 1H), 6.87 (td, J=7.5, 5.6 Hz, 2H), 6.83 (s, 1H), 6.80 (s, 1H), 5.62 (br-s, 1H), 5.42 (br-s, 1H), 4.53-4.35 (m, 6H), 4.19 (app-dtd, J=17.3, 10.9, 6.4 Hz, 2H), 4.00-3.90 (m, 2H), 3.68 (s, 3H), 3.65 (s, 3H), 3.62 (s, 3H), 3.59 (s, 3H), 3.55-3.40 (m, 2H), 2.96 (td, J=11.7, 5.3 Hz, 2H), 2.58-2.44 (m, 2H), 2.39 (ddd, J=15.5, 9.9, 6.1 Hz, 2H), 2.19 (ddd, J=12.3, 4.9, 2.4 Hz, 2H), 1.86 (dd, J=12.0, 4.9 Hz, 1H), 1.77 (dd, J=12.0, 5.1 Hz, 1H), 1.65 (td, J=11.9, 8.1 Hz, 1H), 1.54 (td, J=11.9, 8.5 Hz, 1H), 1.24-0.94 (m, 8), 0.00 (s, 9H), −0.01 (s, 9H), −0.02 (s, 9H), −0.04 (s, 9H). See FIG. 1 for ¹H NMR spectrum.

¹³C NMR (100 MHz, C₆D₆, 70° C.): δ 156.4, 156.2, 155.3 (2C), 154.9, 154.8 (2C), 153.7, 144.5, 144.4, 142.7, 142.6, 141.0, 140.8, 135.0, 134.8, 130.3, 130.2 (2C), 130.0, 126.2, 126.0 (2C), 125.8 (2C), 125.2, 123.6, 123.5, 119.5, 119.2, 116.8, 116.6, 89.9, 89.8, 81.8, 81.6, 79.8, 79.5, 71.1, 70.9, 65.7, 65.5, 64.4, 64.2, 52.4 (4C), 46.0 (2C), 44.6 (2C), 37.1, 36.7 (2C), 36.4, 18.2 (2C), 18.0, 17.9, −1.5 (4C).

FTIR (thin film) cm⁻¹: 3228 (w), 2954 (m), 1700 (s), 1457 (m), 838 (m).

HRMS (ESI) (m/z): calculated for C₇₂H₁₀₀N₁₄NaO₁₈SSi₄ [M+Na]⁺: 1615.6030. found: 1615.6162.

[α]_{D}²⁴: +130 (c=0.59, CH₂Cl₂).

TLC (70% ethyl acetate in hexanes), Rf: 0.21 (UV, CAM).

### Sulfur Extrusion

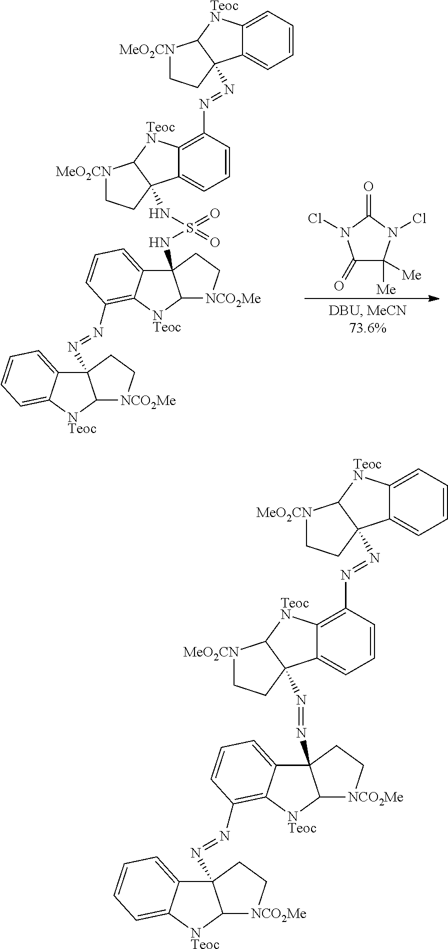

To a solution of mixed sulfamide (766 mg, 481 μmol, 1 equiv) in acetonitrile (24.1 mL) at 22° C. was added via syringe 1,8-diazabicyclo[5.4.0]undec-7-ene (DBU, 215 μL, 1.44 mmol, 3.00 equiv) followed immediately by 1,3-dichloro-5,5-dimethylhydantoin (236 mg, 1.20 mmol, 2.50 equiv) in a single portion. After 1 h, the mixture was diluted with dichloromethane (10 mL) and was washed with a saturated aqueous potassium carbonate-water solution (1:1, 30 mL). The aqueous layer was extracted with dichloromethane (3×30 mL). The combined organic extracts were dried over anhydrous sodium sulfate, were filtered, and were concentrated under reduced pressure. The resulting residue was purified by flash column chromatography on silica gel (eluent: 35→60% ethyl acetate in hexanes) to afford unsymmetrical cyclotryptamine tetramer diazene (541 mg, 73.6%) as a bright yellow amorphous gum.

As a result of the slow conformational equilibration at ambient temperature, NMR spectra were collected at elevated temperature. Structural assignments were made using additional information from gCOSY, HSQC, and HMBC experiments also collected at elevated temperature.

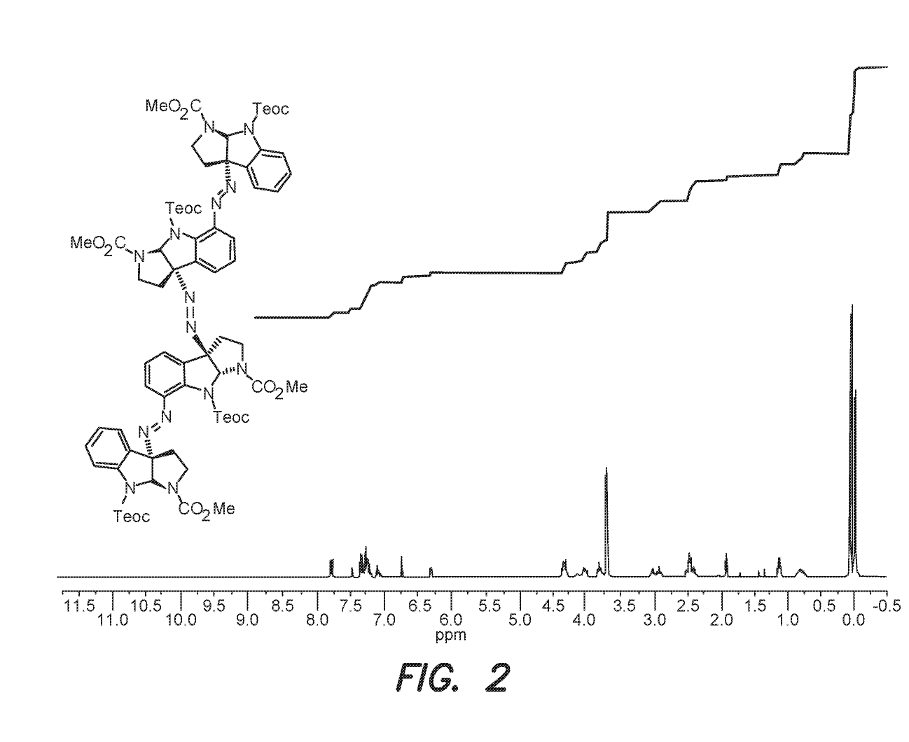

¹H NMR (400 MHz, CD₃CN, 50° C.): δ 7.77 (d, J=8.1 Hz, 2H), 7.48 (d, J=8.9 Hz, 1H), 7.387.18 (m, 9H), 7.09 (app-dtd, J=1.0, 7.5, 11.6 Hz, 2H), 6.74 (app-s, 2H), 6.32 (app-d, J=1.7 Hz, 1H), 6.30 (app-d, J=2.2 Hz, 1H), 4.404.26 (m, 4H), 4.193.94 (m, 4H), 3.893.73 (m, 4H), 3.70 (s, 6H), 3.69 (s, 3H), 3.68 (s, 3H), 3.082.98 (m, 2H), 2.93 (td, J=5.6, 11.4 Hz, 2H), 2.562.35 (m, 8H), 1.181.07 (m, 4H), 0.920.69 (m, 4H), 0.07 (s, 9H), 0.05 (s, 9H), 0.01 (s, 9H), −0.01 (s, 9H). See FIG. 2 for ¹H NMR spectrum.

¹³C NMR (100 MHz, CD₃CN, 50° C.): δ 156.3, 156.2, 156.0 (2C), 155.3, 155.2, 154.5 (2C), 144.7, 144.6, 143.4, 143.2, 141.4, 141.0, 134.7 (2C), 131.1 (3C), 130.7, 127.7, 127.6, 127.1 (2C), 126.8, 126.7, 124.6 (2C), 120.3, 119.9, 117.1, 116.9, 90.3, 90.1, 89.5 (2C), 81.8, 81.7, 79.8, 79.7, 65.8, 65.7, 65.2 (2C), 53.4 (2C), 53.3 (2C), 47.0, 46.9, 46.7 (2C), 37.4, 37.1, 33.7 (2C), 18.7 (4C), −1.1 (4C).

FTIR (thin film) cm⁻¹: 2954 (m), 1717 (s), 1448 (w), 1395 (m).

HRMS (ESI) (m/z): calculated for C₇₂H₉₈N₁₄NaO₁₆SSi₄ [M+Na]⁺: 1549.6255. found: 1549.6665.

[α]_{D}²⁴: +145 (c=0.62, CH₂Cl₂).

TLC (60% ethyl acetate in hexanes), Rf: 0.33 (UV, CAM).

### Csp3-Csp3 Bond Formation

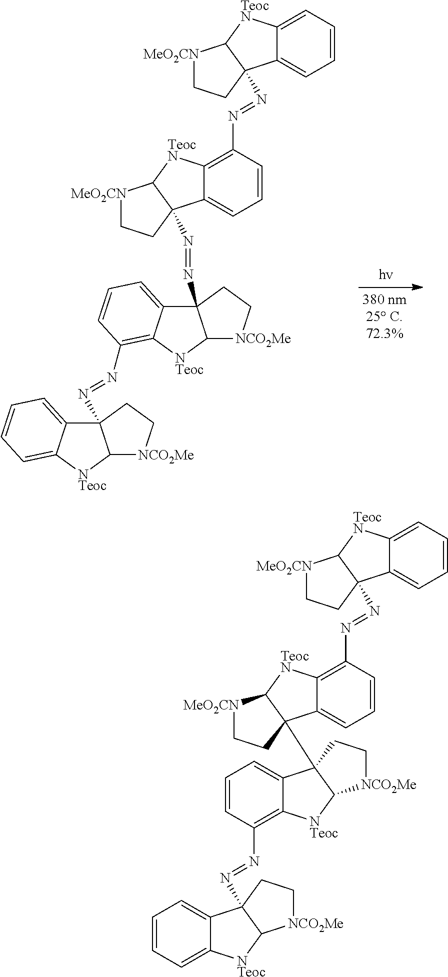

A solution of unsymmetrical tetramer diazene (541 mg, 354 μmol, 1 equiv) in dichloromethane (30 mL) was concentrated under reduced pressure in a 2 L round bottom flask to provide a thin film of diazene coating the flask. The flask was back filled with argon and irradiated in a Rayonet photoreactor equipped with 16 radially distributed (r=12.7 cm) 25 W lamps (λ=380 nm) at 25° C. After 24 h, the lamps were turned off and the resulting residue was purified by flash column chromatography on silica gel (eluent: 30→70% ethyl acetate in hexanes) to afford the cyclotryptamine tetramer (384 mg, 72.3%) as a bright yellow amorphous gum.

As a result of the slow conformational equilibration at ambient temperature, NMR spectra were collected at elevated temperature.

¹H NMR (400 MHz, CD₃CN, 70° C.): δ 7.78 (app-t, J=7.2 Hz, 2H), 7.49 (app-t, J=6.8 Hz, 1H), 7.377.26 (m, 3H), 7.217.14 (m, 2H), 7.147.01 (m, 4H), 6.94 (br-s, 2H), 6.75 (dd, J=6.5, 10.7 Hz, 2H), 6.22 (app-br-s, 2H), 4.414.26 (m, 4H), 4.133.93 (m, 4H), 3.933.78 (m, 2H), 3.783.65 (m, 14H), 3.092.94 (m, 2H), 2.802.66 (m, 2H), 2.572.34 (m, 4H), 2.26 (app-br-s, 4H), 1.201.06 (m, 4H), 1.010.79 (m, 4H), 0.13-0.01 (m, 36H).

¹³C NMR (100 MHz, CD₃CN, 70° C.): δ 156.5, 156.4, 155.9, 155.8, 154.9 (2C), 154.8 (2C), 145.0, 144.9, 143.4, 143.3, 141.3, 140.8, 136.9 (2C), 131.4, 131.3, 131.2, 131.0, 127.4, 127.3, 127.0 (2C), 126.9, 124.8, 124.7, 119.8, 119.4, 117.2 (2C), 90.4 (2C), 82.0, 81.9, 80.5, 80.0, 66.2 (2C), 65.4, 65.3, 62.3 (2C), 53.5 (2C), 53.5 (2C), 47.2, 47.1, 46.7 (2C), 37.8, 37.6, 34.7, 34.6, 19.1 (3C), 19.0, −0.8 (2C), −0.9 (2C).

FTIR (thin film) cm⁻¹: 2954 (m), 1717 (s), 1457 (m), 1251 (w).

HRMS (ESI) (m/z): calculated for C₇₂H₉₈N₁₂NaO₁₆SSi₄ [M+Na]⁺: 1521.6193. found: 1521.6283.

[α]_{D}²⁴: +155 (c=0.55, CH₂Cl₂).

TLC (50% ethyl acetate in hexanes), Rf: 0.13 (UV, CAM).

### Csp2-Cs3 Bond Formation

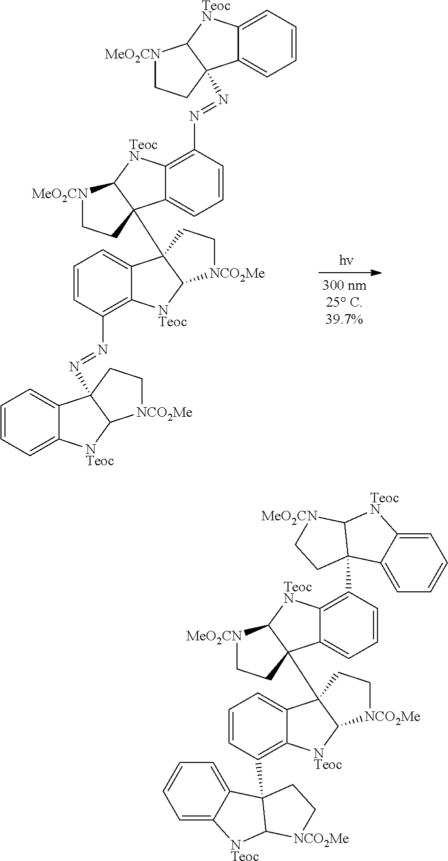

A solution of unsymmetrical tetramer diazene (17.3 mg, 11.5 μmol, 1 equiv) in dichloromethane (5 mL) was concentrated under reduced pressure in a 100 mL round bottom flask to provide a thin film of diazene coating the flask. The flask was back filled with argon and irradiated in a Rayonet photoreactor equipped with 16 radially distributed (r=12.7 cm) 25 W lamps (λ=300 nm) at 25° C. After 24 h, the lamps were turned off and the resulting residue was purified by flash column chromatography on silica gel (eluent: 25→50% ethyl acetate in hexanes) to afford the cyclotryptamine tetramer (6.60 mg, 39.7%) as an off-white amorphous gum.

As a result of the slow conformational equilibration at ambient temperature, NMR spectra were collected at elevated temperature.

### Teoc Deprotection

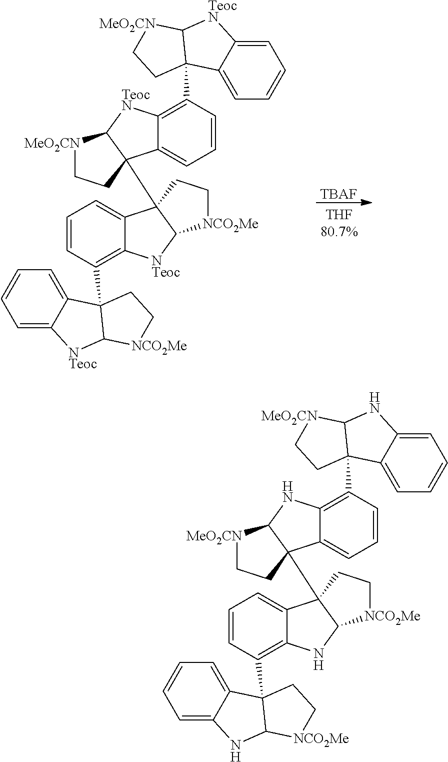

Tetrabutylammonium fluoride (1M in THF, 60.0 μL, 60.0 μmol, 20.0 equiv) was added dropwise to dry cyclotryptamine tetramer (4.70 mg, 3.00 μmol, 1 equiv) at 22° C. After 4.5 h, the reaction mixture was concentrated under reduced pressure. The resulting residue was purified by flash column chromatography on silica gel (eluent: 30→40% acetone in hexanes) to afford deprotected cyclotryptamine tetramer (2.10 mg, 80.7%).

### Carbamate Reduction to (−)-Quadrigemine C

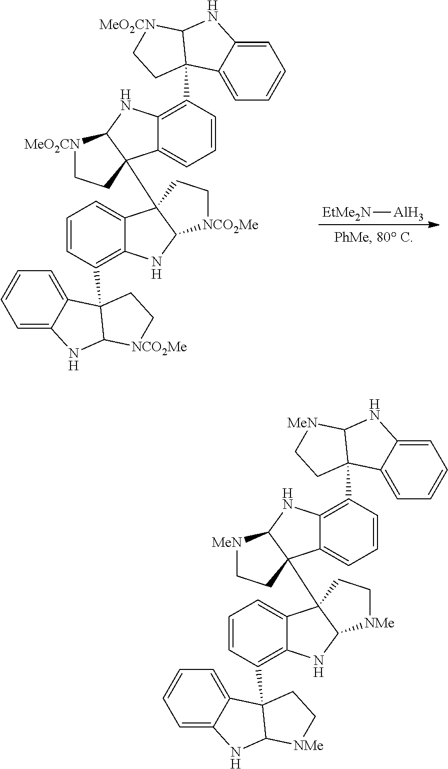

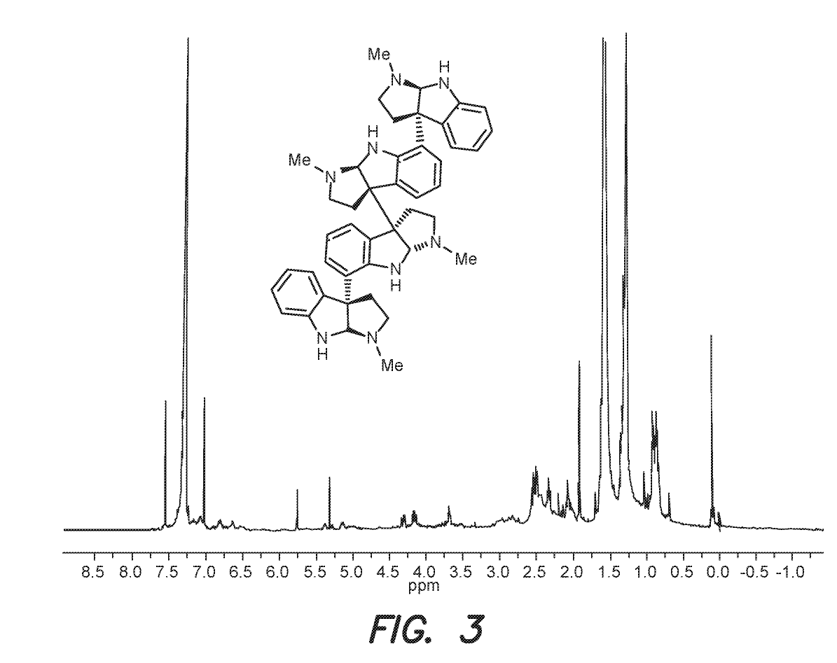

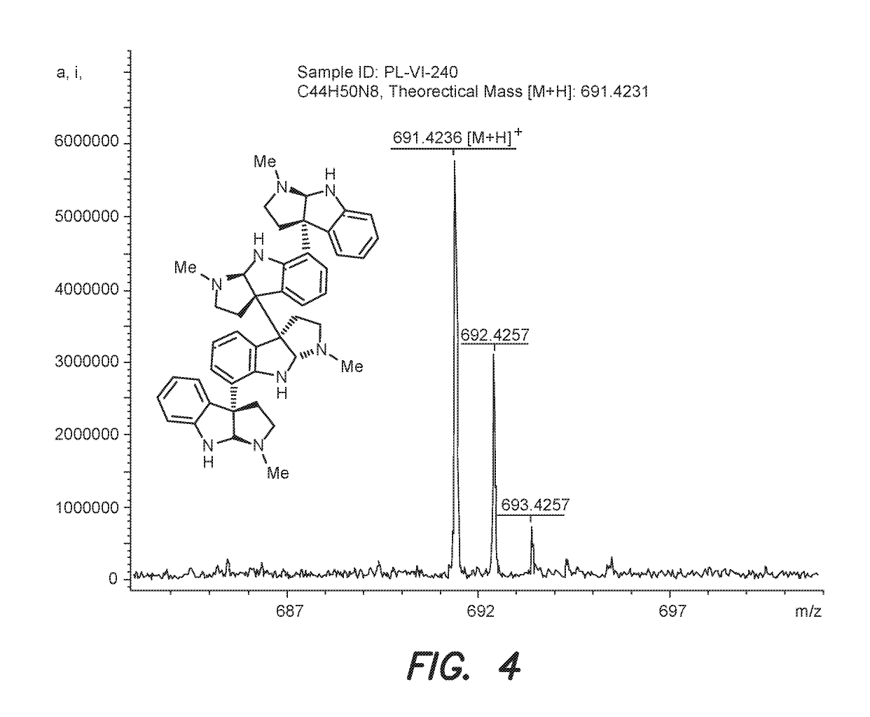

Cyclotryptamine tetramer (0.70 mg, 10 μmol, 1 equiv) was azeotropically dried from anhydrous benzene (2×1 mL) and the residue was dissolved in toluene (50 μL). A solution of alane N,N-dimethylethylamine complex in toluene (0.5 M, 0.15 mL, 60 μmol, 60 equiv) was added via syringe at 23° C. The reaction flask was sealed and heated to 80° C. After 1 h, the reaction mixture was allowed to cool to 23° C. and excess reducing reagent was quenched by the addition of saturated aqueous sodium sulfate solution (0.10 mL). The resulting heterogeneous mixture was stirred for 10 min and then solid anhydrous sodium sulfate was added. The mixture was filtered through a plug of Celite and the filter cake was rinsed with ethyl acetate (5 mL). The filtrate was concentrated under reduced pressure. The resulting residue was purified by flash column chromatography on silica gel (eluent: 30%→100% CMA in chloroform) to afford quadrigemine C (0.28 mg, 40%). See FIG. 3 for ¹H NMR spectrum and FIG. 4 for mass spectral data.

### Example 2. Synthesis of (−)-hodgkinsine

### Sulfamide Formation

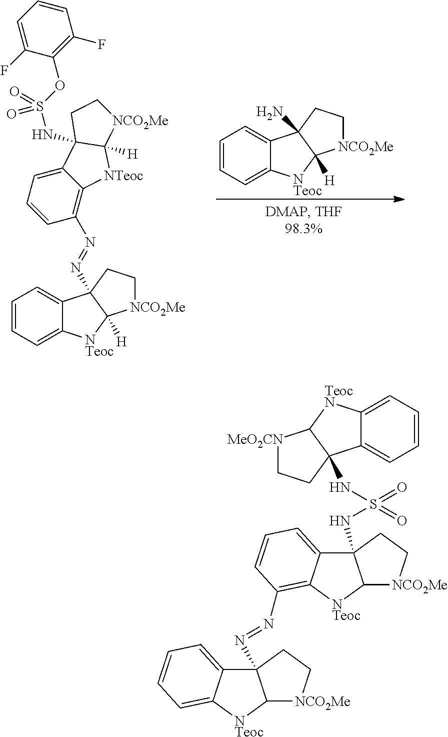

A sample of 4-(dimethylamino)pyridine (109 mg, 891 μmol, 2.20 equiv) was added to a solution of cyclotryptamine diazene sulfamate ester (388 mg, 405 μmol, 1 equiv) and cyclotryptamine amine (168 mg, 446 μmol, 1.10 equiv) in tetrahydrofuran (4.10 mL) at 22° C. After 24 h, the bright yellow solution was concentrated under reduced pressure. The resulting residue was purified by flash column chromatography on silica gel (eluent: 20%→70% ethyl acetate in hexanes) to afford cyclotryptamine trimer (480 mg, 98.3%) as a bright yellow amorphous gum.

As a result of the slow conformational equilibration at ambient temperature, NMR spectra were collected at elevated temperature. Structural assignments were made using additional information from gCOSY, HSQC, and HMBC experiments also collected at elevated temperature.

¹³C NMR (100 MHz, C₆D₆, 70° C.): δ 156.0, 155.3, 155.2, 154.9, 154.4, 153.9, 144.6, 144.1, 142.8, 141.1, 134.6, 130.9, 130.5, 130.3, 126.1, 125.8, 125.3, 124.6, 123.9, 123.6, 119.4, 117.6, 116.8, 89.9, 82.2, 80.2 (2C), 71.4, 71.3, 65.5, 64.8, 64.3, 52.4 (3C), 46.1, 45.0, 44.7, 37.2, 36.7, 36.2, 18.2 (2C), 18.1, −1.5 (3C).

FTIR (thin film) cm⁻¹: 3228 (m), 2955 (m), 1715 (s), 1402 (m).

HRMS (ESI) (m/z): calculated for C₅₄H₇₆N₁₀NaO₁₄SSi₃ [M+Na]⁺: 1227.4463. found: 1227.4462.

[α]_{D}²⁴: −83 (c=0.64, CH₂Cl₂).

TLC (60% ethyl acetate in hexanes), Rf: 0.28 (UV, CAM).

### Sulfur Extrusion

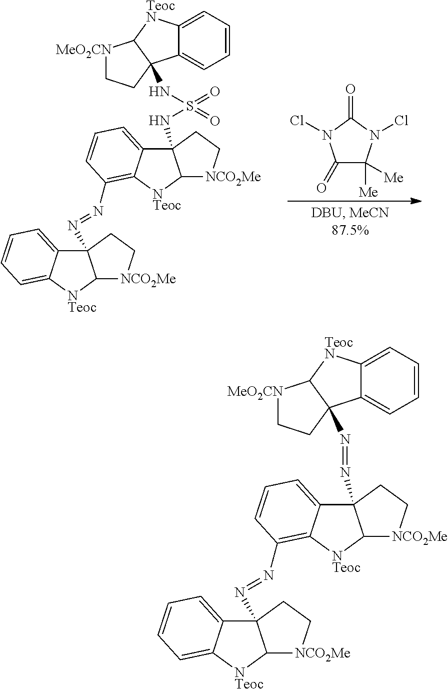

To a solution of mixed sulfamide (480 mg, 398 μmol, 1 equiv) in acetonitrile (20.0 mL) at 22° C. was added via syringe 1,8-diazabicyclo[5.4.0]undec-7-ene (DBU, 178 μL, 1.19 mmol, 3.00 equiv) followed immediately by 1,3-dichloro-5,5-dimethylhydantoin (196 mg, 995 μmol, 2.50 equiv) in a single portion. After 1 h, the mixture was diluted with dichloromethane (20 mL) and was washed with a saturated aqueous potassium carbonate-water solution (1:1, 30 mL). The aqueous layer was extracted with dichloromethane (3×30 mL). The combined organic extracts were dried over anhydrous sodium sulfate, were filtered, and were concentrated under reduced pressure. The resulting residue was purified by flash column chromatography on silica gel (eluent: 20→50% ethyl acetate in hexanes) to afford unsymmetrical cyclotryptamine trimer diazene (397 mg, 87.5%) as a bright yellow amorphous gum.

As a result of the slow conformational equilibration at ambient temperature, NMR spectra were collected at elevated temperature. Structural assignments were made using additional information from gCOSY, HSQC, and HMBC experiments also collected at elevated temperature.

¹H NMR (400 MHz, CD₃CN, 50° C.): δ 7.77 (d, J=8.0 Hz, 1H), 7.70 (d, J=8.0 Hz, 1H), 7.427.19 (m, 6H), 7.12 (app-t, J=6.4 Hz, 1H), 7.08 (app-t, J=7.5 Hz, 1H), 7.02 (app-t, J=7.4 Hz, 1H), 6.73 (s, 1H), 6.46 (s, 1H), 6.39 (s, 1H), 4.384.20 (m, 4H), 4.204.08 (m, 1H), 4.01 (br-dd, J=7.4, 10.3 Hz, 1H), 3.933.76 (m, 3H), 3.69 (app-s, 6H), 3.65 (s, 3H), 3.082.89 (m, 2H), 2.57 (dd, J=5.1, 12.5 Hz, 1H), 2.532.38 (m, 3H), 2.372.20 (m, 2H), 1.171.03 (m, 4H), 0.900.67 (m, 2H), 0.07 (app-s, 18H), −0.03 (s, 9H).

¹³C NMR (100 MHz, CD₃CN, 50° C.): δ 156.2, 156.0 (2C), 155.3, 154.4, 154.3, 144.6, 144.5, 143.1, 141.3, 134.8, 131.2, 131.1, 130.6, 130.1, 127.7, 127.1, 126.6, 125.9, 124.6, 124.5, 119.8, 117.2, 117.0, 90.2, 89.6, 89.3, 81.8, 79.7, 79.6, 65.7, 65.2, 65.1, 53.3 (2C), 53.2, 46.9, 46.6 (2C), 36.9, 36.0, 33.6, 18.6 (3C), −1.2 (2C), −1.3.

FTIR (thin film) cm⁻¹: 2954 (m), 1713 (s), 1401 (m), 1552 (w).

HRMS (ESI) (m/z): calculated for C₅₄H₇₄N₁₀NaO₁₂Si₃ [M+Na]⁺: 1161.4688. found: 1161.4673.

[α]_{D}²⁴: −86 (c=0.61, CH₂Cl₂).

TLC (50% ethyl acetate in hexanes), Rf: 0.23 (UV, CAM).

### Csp3-Csp3 Bond Formation

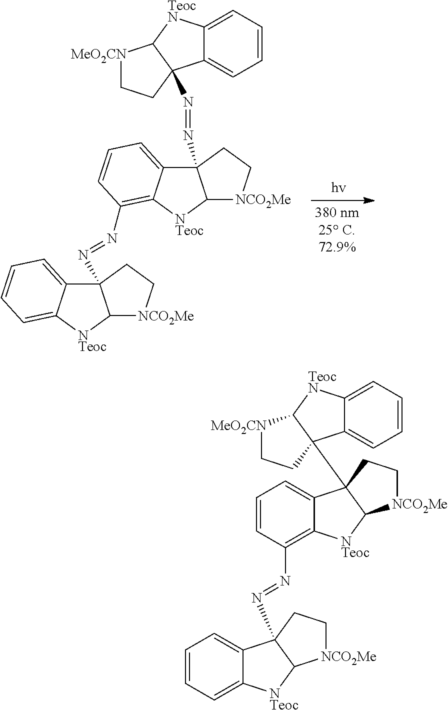

A solution of trimer diazene (397 mg, 348 μmol, 1 equiv) in dichloromethane (30 mL) was concentrated under reduced pressure in a 1 L round bottom flask to provide a thin film of diazene coating the flask. The flask was back filled with argon and irradiated in a Rayonet photoreactor equipped with 16 radially distributed (r=12.7 cm) 25 W lamps (λ=380 nm) at 25° C. After 15 h, the lamps were turned off and the resulting residue was purified by flash column chromatography on silica gel (eluent: 20→60% ethyl acetate in hexanes) to afford the cyclotryptamine trimer (282 mg, 72.9%) as a bright yellow amorphous gum.

As a result of the slow conformational equilibration at ambient temperature, NMR spectra were collected at elevated temperature.

¹H NMR (400 MHz, CD₃CN, 60° C.): δ 7.75 (d, J=8.0 Hz, 1H), 7.66 (d, J=8.0 Hz, 1H, 7.387.27 (m, 2H), 7.277.15 (m, 4H), 7.06 (app-t, J=7.5 Hz, 1H), 6.77 (app-t, J=7.8 Hz, 1H), 6.69 (s, 1H), 6.41 (br-d, J=5.8 Hz, 1H), 6.23 (s, 1H), 6.08 (s, 1H), 4.394.28 (m, 3H), 4.21 (td, J=7.0, 10.5 Hz, 1H), 3.99 (dd, J=7.8, 11.1 Hz, 1H), 3.943.85 (m, 1H), 3.82 (dd, J=7.7, 11.0 Hz, 1H), 3.75 (dd, J=7.9, 10.9 Hz, 1H), 3.723.62 (m, 10H), 3.01 (td, J=5.6, 11.6 Hz, 1H), 2.75 (app-dtd, J=5.5, 11.5, 14.1 Hz, 2H), 2.47 (td, J=7.8, 12.1 Hz, 1H), 2.35 (app-ddd, J=7.0, 12.2, 13.5 Hz, 3H), 2.282.16 (m, 2H), 1.181.04 (m, 4H), 0.940.81 (m, 2H), 0.10 (s, 9H), 0.07 (s, 9H), 0.06 (s, 9H).

¹³C NMR (100 MHz, CD₃CN, 60° C.): δ 156.4, 155.9, 155.8, 154.6, 154.4, 154.3, 144.8, 144.5, 143.0, 141.2, 137.3, 132.2, 131.1, 130.8, 130.4, 127.0, 126.9, 125.3, 124.8, 124.6, 119.2, 117.3, 117.1, 90.2, 81.7, 80.3, 79.9, 65.8, 65.4, 65.3, 62.7, 61.8, 53.4 (2C), 53.3, 47.1, 46.9, 46.5, 37.2, 36.2, 34.2, 18.9, 18.8, 18.6, −1.1 (3C).

FTIR (thin film) cm⁻¹: 2954 (m), 1717 (s), 1448 (w), 1400 (w).

HRMS (ESI) (m/z): calculated for C₅₄H₇₄N₈NaO₁₂Si₃ [M+Na]⁺: 1133.4626. found: 1133.4601.

[α]_{D}²⁴: 162 (c=0.54, CH₂Cl₂).

TLC (50% ethyl acetate in hexanes), Rf: 0.19 (UV, CAM).

### Csp2-Csp3 Bond Formation

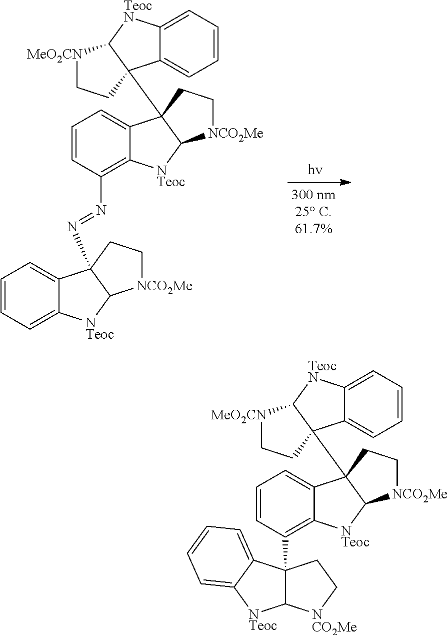

A solution of trimer diazene (141 mg, 127 μmol, 1 equiv) in dichloromethane (15 mL) was concentrated under reduced pressure in a 2 L round bottom flask to provide a thin film of diazene coating the flask. The flask was back filled with argon and irradiated in a Rayonet photoreactor equipped with 16 radially distributed (r=12.7 cm) 25 W lamps (λ=300 nm) at 25° C. After 15 h, the lamps were turned off and the resulting residue was purified by flash column chromatography on silica gel (eluent: 30→40% ethyl acetate in hexanes) to afford the cyclotryptamine trimer (84.9 mg, 61.7%) as an off-white solid.

As a result of the slow conformational equilibration at ambient temperature, NMR spectra were collected at elevated temperature.

¹H NMR (400 MHz, CD₃CN, 60° C.): δ 7.74 (d, J=8.0 Hz, 1H0, 7.70 (d, J=8.1 Hz, 1H), 7.29 (app-dd, J=7.2, 14.2 Hz, 2H), 7.22 (app-t, J=7.8 Hz, 1H), 7.137.00 (m, 2H), 6.91 (d, J=7.5 Hz, 1H), 6.78 (d, J=7.5 Hz, 1H), 6.69 (s, 1H), 6.48 (d, J=7.9 Hz, 1H), 6.40 (br-d, J=5.8 Hz, 1H), 6.36 (s, 1H), 6.04 (s, 1H), 4.504.34 (m, 2H), 4.334.22 (m, 2H), 3.94 (td, J=6.0, 11.4 Hz, 1H), 3.80 (dd, J=7.4, 11.1 Hz, 1H), 3.763.56 (m, 13H), 2.95 (td, J=8.3, 11.8 Hz, 1H), 2.792.36 (m, 3H), 2.352.15 (m, 3H), 2.09 (td, J=8.5, 11.9 Hz, 1H), 1.251.06 (m, 4H), 1.04-0.81 (m, 2H), 0.12 (s, 9H), 0.08 (s, 9H), 0.06 (s, 9H).

¹³C NMR (100 MHz, CD₃CN, 60° C.): δ 156.2, 156.0, 155.8, 155.0 (2C), 154.4, 144.5, 144.4, 142.7, 139.5, 138.2, 134.8, 133.0, 132.3, 130.4, 129.8, 127.4, 125.5, 125.4, 125.3, 125.0, 124.3, 117.6, 117.2, 83.0, 80.7, 80.2, 66.1, 65.4, 65.0, 61.7 (2C), 61.5, 53.4, 53.2, 53.1, 46.9 (2C), 46.1, 35.7, 34.3, 33.4, 19.0 (2C), 18.7, −1.0 (2C), −1.1.

FTIR (thin film) cm⁻¹: 2954 (m), 1716 (s), 1447 (w), 1400 (m).

HRMS (ESI) (m/z): calculated for C₅₄H₇₄N₆NaO₁₂Si₃ [M+Na]⁺: 1105.4565. found: 1105.4539.

[α]_{D}²⁴: −35 (c=0.57, CH₂C₁₂).

TLC (50% ethyl acetate in hexanes), Rf: 0.28 (UV, CAM).

M.p.: 108-110 (CH₂Cl₂).

### Teoc Deprotection

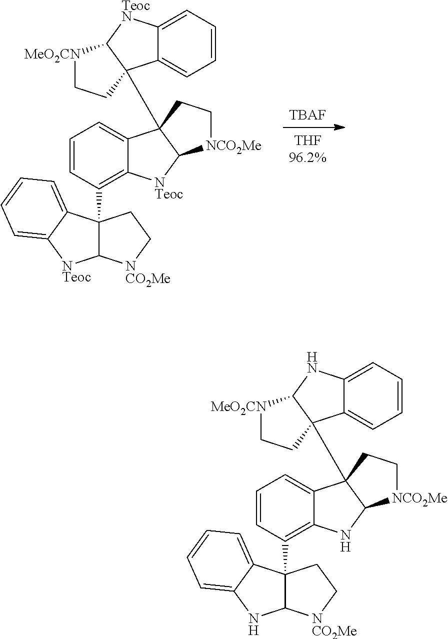

Tetrabutylammonium fluoride (1M in THF, 1.80 mL, 1.80 mmol, 15.0 equiv) was added dropwise to dry cyclotryptamine trimer (130 mg, 120 μmol, 1 equiv) at 22° C. After 2 h, the reaction mixture was concentrated under reduced pressure. The resulting residue was purified by flash column chromatography on silica gel (eluent: 20→40% acetone in hexanes) to afford deprotected cyclotryptamine trimer (75.1 mg, 96.2%).

FTIR (thin film) cm⁻¹: 3335 (m), 2955 (m), 1700 (s), 1608 (w), 1457 (s).

HRMS (ESI) (m/z): calculated for C₃₆H₃₉N₆O₆ [M+H]⁺: 651.2926. found: 651.2916.

[α]_{D}²⁴: +187 (c=0.54, CH₂Cl₂).

TLC (70% ethyl acetate in hexanes), Rf: 0.10 (UV, CAM).

M.p.: 153-155 (CH₂Cl₂).

### Carbamate Reduction to (−)-Hodgkinsine

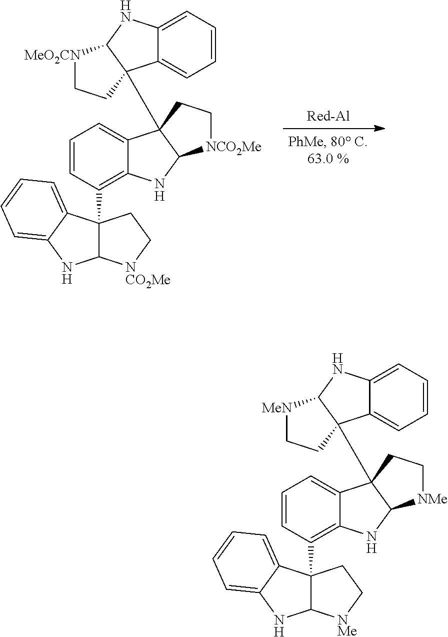

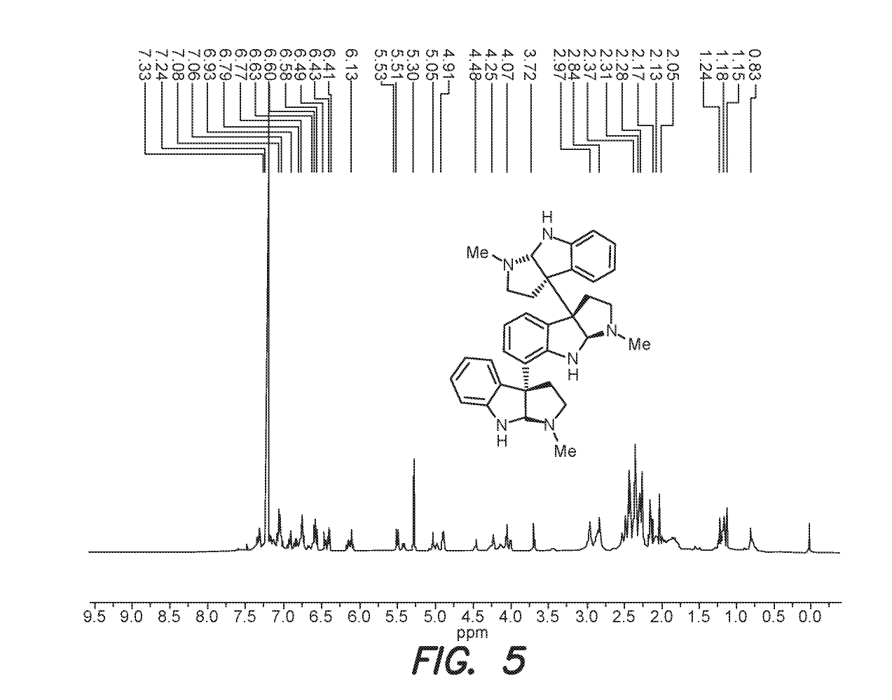

Cyclotryptamine trimer (4.90 mg, 8.00 μmol, 1 equiv) was azeotropically dried from anhydrous benzene (2×1 mL) and the residue was dissolved in toluene (80.0 μL). A solution of sodium bis(2-methoxyethoxy)aluminum hydride in toluene (Red-Al, 70% wt, 53.0 μL, 184 μmol, 23.0 equiv) was added via syringe at 22° C. The reaction flask was sealed and heated to 80° C. After 1 h, the reaction mixture was allowed to cool to 22° C. and excess reducing reagent was quenched by the addition of saturated aqueous sodium sulfate solution (100 μL). The resulting heterogeneous mixture was stirred for 10 min and then solid anhydrous sodium sulfate was added. The mixture was filtered through a plug of Celite and the filter cake was rinsed with ethyl acetate (5 mL). The filtrate was concentrated under reduced pressure. The resulting residue was purified by flash column chromatography on silica gel (eluent: 35%→50% CMA in chloroform) to afford hodgkinsine (2.60 mg, 63.0%). See FIG. 5 for ¹H NMR spectrum.

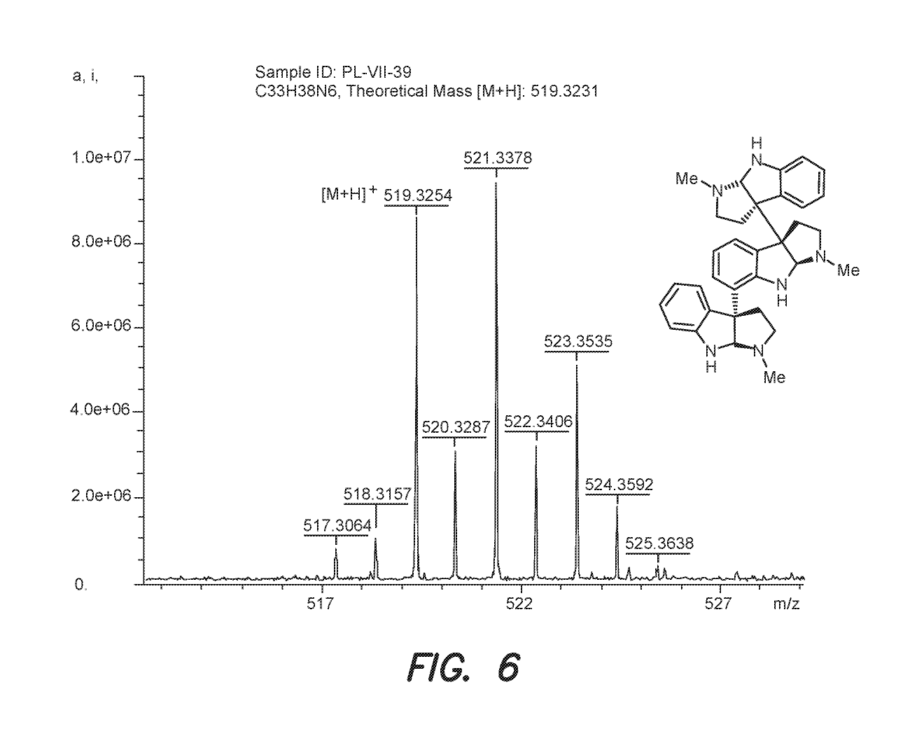

HRMS (DART) (m/z): calculated for C₃₃H₃₉N₆ [M+H]⁺: 519.3231. found: 519.3254 (FIG. 6).

### Example 3. Building Block Synthesis

### Formation of Azide

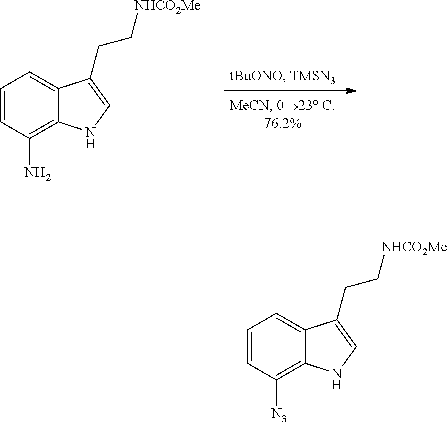

To a solution of methyl (2-(7-amino-1H-indol-3-yl)ethyl)carbamate (998 mg, 4.28 mmol, 1 equiv) in acetonitrile (54.0 mL) at 0° C. were sequentially added tert-butyl nitrite (825 μL, 6.24 mmol, 1.50 equiv) and azidotrimethylsilane (1.01 mL, 7.28 mmol, 1.70 equiv). The reaction mixture was allowed to warm to 22° C. After 24 h, the reaction mixture was concentrated under reduced pressure. The resulting residue was purified by flash column chromatography on silica gel (eluent: 30%→40% ethyl acetate in hexanes) to afford cyclotryptamine azide (846 mg, 76.2%).

¹H NMR (400 MHz, CDCl₃, 20° C.): δ 8.29 (s, 1H, NH), 7.39 (d, J=7.9 Hz, 1H, C₄H), 7.13 (app-t, J=7.8 Hz, 1H, C₅H), 7.02 (br-s, 1H, C₈ₐH), 6.98 (d, J=7.5 Hz, 1H, C₆H), 4.80 (s, 1H, NHCO₂CH₃), 3.67 (s, 3H, NHCO₂CH₃), 3.51 (dd, J=6.1, 12.4 Hz, 2H, C₂H₂), 2.95 (t, J=6.8 Hz, 2H, C₃H₂).

¹³C NMR (100 MHz, CDCl₃, 20° C.): δ 157.2 (NHCO₂CH₃), 129.1 (C₄ₐ), 128.6 (C₇ₐ), 124.6 (C₇), 122.7 (C₈ₐ), 120.3 (C₅), 115.5 (C₄), 113.6 (C₃ₐ), 110.6 (C₆), 52.2 (NHCO₂CH₃), 41.4 (C₂), 25.9 (C₃).

FTIR (thin film) cm⁻¹: 3322 (s), 2946 (m), 2115 (s), 1700 (s), 1522 (m), 1282 (m).

HRMS (DART) (m/z): calculated for C₁₂H₁₄N₅O₂ [M+H]⁺: 260.1142. found 260.1152.

TLC (50% ethyl acetate in hexanes), Rf: 0.43 (UV, CAM).

M.p.: 87-89 (CH₂Cl₂).

Nitrogen Protection with Teoc

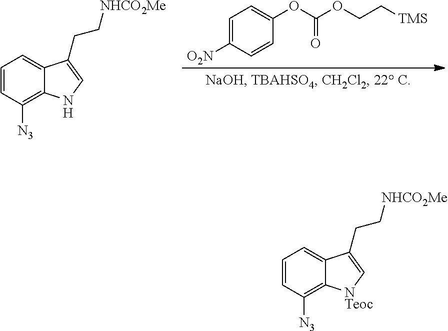

To a solution of methyl (2-(7-azido-1H-indol-3-yl)ethyl)carbamate (1.66 g, 6.40 mmol, 1 equiv), 4-nitrophenyl (2-(trimethylsilyl)ethyl) carbonate (2.72 g, 9.60 mmol, 1.50 equiv) and tetrabutylammonium hydrogensulfate (217 mg, 640 μmol, 10.0 mol %) in dichloromethane (64.0 mL) at 22° C. was added finely powdered sodium hydroxide (768 mg, 19.2 mmol, 3.00 equiv). After 15.5 h, the reaction mixture was washed with aqueous sodium hydroxide (1N, 100 mL) and the aqueous layer was extracted with dichloromethane (3×50 mL). The combined organic layers were dried over anhydrous sodium sulfate, were filtered and were concentrated under reduced pressure. The resulting residue was purified by flash column chromatography on silica gel (eluent: 5%→15% acetone in hexanes) to afford 2-(trimethylsilyl)ethyl-7-azido-3-(2-((methoxycarbonyl)amino)ethyl)-1H-indole-1-carboxylate (2.53 g, 98.0%) as an off-white amorphous gum. Structural assignments were made using additional information from gHSQC and gHMBC experiments.

¹H NMR (400 MHz, CDCl₃, 20° C.): δ 7.42 (s, 1H, C₈ₐH), 7.35 (d, J=7.7 Hz, 1H, C₄H), 7.28 (app-t, J=7.8 Hz, 1H, C₅H), 7.14 (d, J=8.4 Hz, 1H, C₆H), 4.78 (br-s, 1H, NHCO₂CH₃), 4.544.44 (m, 2H, NCO₂CH₂CH₂Si(CH₃)₃), 3.67 (s, 3H, NHCO₂CH₃), 3.49 (dd, J=6.4, 12.9 Hz, 2H, C₂H₂), 2.88 (t, J=6.8 Hz, 2H, C₃H₂), 1.261.17 (m, 2H, NCO₂CH₂CH₂Si(CH₃)₃), 0.09 (s, 9H, NCO₂CH₂CH₂Si(CH₃)₃).

¹³C NMR (100 MHz, CDCl₃, 20° C.): δ 157.1 (NHCO₂CH₃), 150.4 (NCO₂CH₂CH₂Si(CH₃)₃), 133.9 (C₄ₐ), 127.9 (C₇), 126.8 (C₇ₐ), 126.1 (C₈ₐ), 124.1 (C₅), 117.8 (C₃ₐ), 116.0 (C₆), 115.9 (C₄), 66.4 (NCO₂CH₂CH₂Si(CH₃)₃), 52.2 (NCO₂CH₃), 40.6 (C₂), 25.6 (C₃), 17.7 (NCO₂CH₂CH₂Si(CH₃)₃), −1.4 (NCO₂CH₂CH₂Si(CH₃)₃).

FTIR (thin film) cm⁻¹: 3370 (m), 2954 (s), 2115 (s), 1728 (s), 1526 (w).

HRMS (DART) (m/z): calculated for C₁₈H₂₉N₆O₄Si [M+NH₄]⁺: 421.2014. found 421.2005.

TLC (20% ethyl acetate in hexanes), Rf: 0.34 (UV, CAM).

### Cyclization to Cyclotryptamine

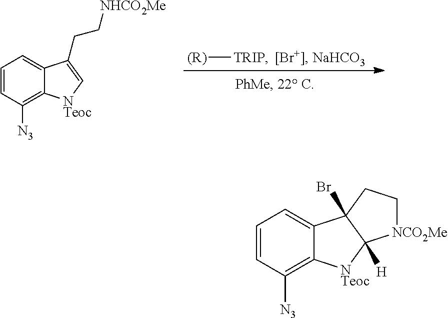

A sample of bromine salt S1₁ (4.32 g, 8.09 mmol, 1.30 equiv) was added to a suspension of tryptamine (2.51 g, 6.22 mmol, 1 equiv), (R)-3,3′-Bis(2,4,6-triisopropyl-phenyl)-1,1′-binaphthyl-2,2′-diyl hydrogen phosphate ((R)-TRIP, 468 mg, 622 μmol, 10.0 mol %), and sodium hydrogen carbonate (2.09 g, 24.9 mmol, 4.00 equiv) in toluene (124 mL) at 22° C. After stirring for 22 h, the yellow suspension was diluted with a saturated aqueous sodium thiosulfate solution (50 mL) and was stirred vigorously for 10 min. The biphasic mixture was further diluted with deionized water (50 mL) and was then extracted with dichloromethane (3×50 mL). The combined organic extracts were dried over anhydrous sodium sulfate, were filtered, and were concentrated under reduced pressure. The resulting residue was purified by flash column chromatography on silica gel (eluent: 5→10% acetone in hexanes) to afford bromocyclotryptamine (2.69 g, 89.7%, 97:3 er) as a colorless amorphous gum. ₂ The enantiomeric ratio was determined by chiral HPLC analysis (Chiralpak IA, 5% iPrOH/95% hexanes, 0.75 mL/min, 254 nm, t_{R} (major)=8.69 min, t_{R} (minor)=11.0 min). Structural assignments were made using additional information from gHSQC and gHMBC experiments. ₁ Xie, W.; Jiang. G.; Liu, H.; Hu, J.; Pan, X.; Zhang, H.; Wan, X.; Lai, Y.; Ma. D. Angew. Chem. Int. Ed. 2013. 52, 12924 (the entirety is herein explicitly incorporate by reference).₂ Further elution with 60% ethyl acetate in hexanes allows for the recovery of the (S)-TRIP catalyst.

¹H NMR (400 MHz, CDCl₃, 20° C.): δ 7.21 (app-t, J=7.6 Hz, 1H, C₅H), 7.16 (dd, J=1.4, 7.6 Hz, 1H, C₄H), 7.03 (d, J=7.5 Hz, 1H, C₆H), 6.31 (br-s, 1H, C₈ₐH), 4.484.29 (m, 2H, NCO₂CH₂CH₂Si(CH₃)₃) 3.74 (s, 3H, NCO₂CH₃), 3.67 (t, J=7.9 Hz, 1H, C₃Hₐ), 2.88-2.67 (m, 3H, C₃H_{b}, C₂H₂), 1.13 (t, J=8.8 Hz, 2H, NCO₂CH₂CH₂Si(CH₃)₃) 0.03 (s, 9H, NCO₂CH₂CH₂Si(CH₃)₃).

¹³C NMR (100 MHz, CDCl₃, 20° C.): δ 154.8 (NCO₂CH₃), 154.3 (NCO₂CH₂CH₂Si(CH₃)₃), 137.4 (C₄ₐ), 133.8 (C₇ₐ), 132.2 (C₇), 127.5 (C₅), 122.4 (C₆), 119.7 (C₄), 86.0 (C₅ₐ), 65.7 (NCO₂CH₂CH₂Si(CH₃)₃), 60.9 (C₃ₐ), 53.0 (NCO₂CH₃), 46.0 (C₃), 39.2 (C₂), 17.7 (NCO₂CH₂CH₂Si(CH₃)₃), −1.4 (NCO₂CH₂CH₂Si(CH₃)₃).

FTIR (thin film) cm⁻¹: 2955 (m), 2116 (s), 1716 (s), 1449 (m), 1311 (m).

HRMS (DART) (m/z): calculated for C₁₈H₂₅N₅O₄Si [M+H]⁺: 482.0854. found 482.0870.

[α]_{D}²⁴: −222 (c=0.62, CH₂Cl₂).

TLC (20% acetone in hexanes), Rf: 0.33 (UV, CAM).

### Formation of 7-Amino Group by Azide Reduction

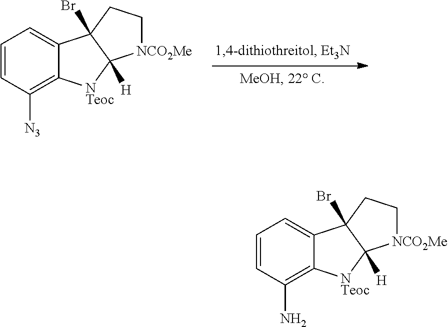

Triethylamine (4.91 mL, 35.2 mmol, 6.30 equiv) was added via syringe to a solution of azide (2.69 g, 5.58 mmol, 1 equiv) and dithiothreitol (4.40 mL, 28.5 mmol, 5.11 equiv) in methanol (56.0 mL). After 15 h, the reaction was diluted with dichloromethane (30 mL) and with a saturated aqueous sodium periodate solution (5 mL). After vigorous stirring for 10 min, the heterogeneous biphasic mixture was washed with saturated aqueous sodium chloride solution (50 mL) then extracted with dichloromethane (3×30 mL). The combined organic extracts were dried over anhydrous sodium sulfate, were filtered, and were concentrated under reduced pressure. The resulting residue was purified by flash column chromatography on silica gel (eluent: 15→25% ethyl acetate in hexanes) to afford cyclotryptamine amine (2.15 g, 84.4%) as a colorless amorphous gum. Structural assignments were made using additional information from gHSQC and gHMBC experiments.

¹H NMR (400 MHz, CDCl₃, 20° C.): δ 7.03 (app-t, J=7.7 Hz, 1H, C₅H), 6.78 (d, J=7.5 Hz, 1H, C₄H), 6.67 (d, J=7.9 Hz, 1H, C₆H), 6.35 (br-s, 1H, C₈ₐH), 4.67 (br-s, 2H, NH₂), 4.504.28 (m, 2H, NCO₂CH₂CH₂Si(CH₃)₃), 3.71 (s, 3H, NCO₂CH₃), 3.64 (br-s, 1H, C₂Hₐ), 2.88-2.75 (m, 2H, C₂H_{b}, C₃Hₐ), 2.752.62 (m, 1H, C₃H_{b}), 1.24-1.06 (m, 2H, NCO₂CH₂CH₂Si(CH₃)₃), −0.07 (s, 9H, NCO₂CH₂CH₂Si(CH₃)₃).

¹³C NMR (100 MHz, CDCl₃, 20° C.): δ 155.3 (2C, NCO₂CH₃, (NCO₂CH₂CH₂Si(CH₃)₃) 138.4 (C₇), 135.5 (C₄ₐ), 128.5 (C₇ₐ), 127.4 (C₅), 119.1 (C₆), 112.7 (C₄), 84.9 (C₅ₐ), 65.5 (NCO₂CH₂CH₂Si(CH₃)₃), 61.9 (C₃ₐ), 52.8 (NCO₂CH₃), 46.1 (C₂), 39.6 (C₃), 17.9 (NCO₂CH₂CH₂Si(CH₃)₃), −1.4 (NCO₂CH₂CH₂Si(CH₃)₃).

FTIR (thin film) cm⁻¹: 3423 (m), 2955 (s), 1700 (s), 1623 (m).

HRMS (DART) (m/z): calculated for C₁₈H₂₇BrN₃O₄Si [M+H]⁺: 456.0949. found 456.0938.

[α]_{D}²⁴: −322 (c=0.53, CH₂Cl₂).

TLC (50% ethyl acetate in hexanes), Rf: 0.51 (UV, CAM).

### Formation of C₇-Sulfonamide and Debromination

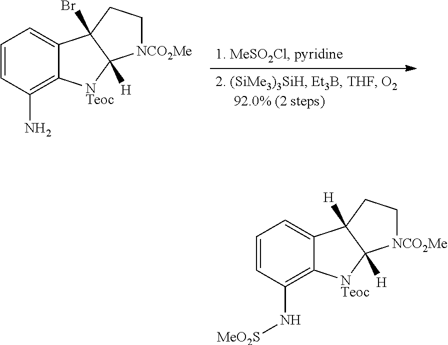

To a solution of cyclotryptamine (2.09 g, 4.58 mmol, 1 equiv) in pyridine (46.0 mL) was added methanesulfonyl chloride (709 μL, 9.16 mmol, 2.00 equiv). After 2 h, the reaction mixture was diluted with dichloromethane (30 mL), washed with saturated aqueous sodium chloride solution (50 mL) then extracted with dichloromethane (3×30 mL). The combined organic extracts were dried over anhydrous sodium sulfate, were filtered, and were concentrated under reduced pressure to afford crude methanesulfonyl cyclotryptamine as a yellow amorphous gum that was used in the next step without further purification.

For characterization purposes, the crude methanesulfonyl cyclotryptamine was purified by flash column chromatography on silica gel (eluent: 15→25% ethyl acetate in hexanes) to afford cyclotryptamine amine as a colorless amorphous gum.

Structural assignments were made using additional information from gCOSY, gHSQC, and gHMBC experiments.

¹H NMR (400 MHz, CDCl₃, 20° C.): δ 9.09 (s, 1H, NHSO₂CH₃), 7.61-7.47 (m, 1H, C₆H), 7.317.21 (m, 2H, C₄H, C₅H), 6.34 (s, 1H, C₈ₐH), 4.51-4.29 (m, 2H, NCO₂CH₂CH₂Si(CH₃)₃), 3.66 (app-s, 4H, NCO₂CH₃, C₂Hₐ), 2.91-2.82 (m, 1H, C₃Hₐ), 2.80-2.71 (m, 5H, NHSO₂CH₃, C₂H_{b}, C₃H_{b}), 1.27-1.09 (m, 2H, NCO₂CH₂CH₂Si(CH₃)₃) 0.07 (s, 9H, NCO₂CH₂CH₂Si(CH₃)₃).

¹³C NMR (100 MHz, CDCl₃, 20° C.): δ 155.9 (NCO₂CH₂CH₂Si(CH₃)₃), 154.2 (NCO₂CH₃), 136.0 (C₄ₐ), 134.3 (C₇ₐ), 127.9 (2C, C₄ or C₅, C₇), 126.9 (C₆), 120.8 (C₄ or C₅), 84.9 (C₈ₐ), 66.9 (NCO₂CH₂CH₂Si(CH₃)₃), 60.1 (C₃ₐ), 52.9 (NCO₂CH₃), 46.1 (C₂), 39.2 (NHSO₂CH₃), 39.0 (C₃), 17.9 (NCO₂CH₂CH₂Si(CH₃)₃), 1.4 (NCO₂CH₂CH₂Si(CH₃)₃).

FTIR (thin film) cm⁻¹: 3175 (w), 2956 (m), 1716 (s), 1688 (s), 1161 (s).

HRMS (ESI) (m/z): calculated for C₁₉H₂₈BrN₃NaO₆SSi [M+Na]⁺: 556.0544. found 556.0550.

[α]_{D}²⁴: −259 (c=0.61, CH₂Cl₂).

TLC (50% ethyl acetate in hexanes), Rf: 0.38 (UV, CAM).

C3a-bromide Reduction

Triethylborane (1.0 M in THF, 916 μL, 916 μmol, 0.200 equiv) was added via syringe to a solution of crude methanesulfonyl cyclotryptamine and tris(trimethylsilyl)silane (4.23 mL, 13.7 mmol, 3.00 equiv) in tetrahydrofuran (46.0 mL) at 22° C. under an air atmosphere. After 1 h, the reaction mixture was washed with a saturated aqueous sodium bicarbonate solution (50 mL), then extracted with dichloromethane (3×50 mL). The combined organic extracts were dried over anhydrous sodium sulfate, were filtered, and were concentrated under reduced pressure to yield a colorless semi-solid suspended in a colorless oil. The resulting residue was purified by flash column chromatography on silica gel (eluent: 0→20% acetone in hexanes) to afford the reduced cyclotryptamine (1.92 g, 92.0%) as a colorless amorphous gum over two steps.

Structural assignments were made using additional information from gCOSY, gHSQC, and gHMBC experiments.

¹H NMR (400 MHz, CDCl₃, 20° C.): δ 9.08 (s, 1H, NHSO₂CH₃), 7.42 (d, J=8.0 Hz, 1H, C₆H), 7.20 (app-t, J=7.8 Hz, 1H, C₅H), 7.10 (app-dt, J=1.1, 7.5 Hz, 1H, C₄H), 6.32 (d, J=5.7 Hz, 1H, C₅ₐH), 4.46-4.27 (m, 2H, NCO₂CH₂CH₂Si(CH₃)₃), 4.06 (app-br-s, 1H, C₃ₐH), 3.65 (app-s, 4H, NCO₂CH₃, C₂Hₐ), 2.79 (app-dd, J=9.6, 18.6 Hz, 1H, C₂H_{b}), 2.68 (s, 3H, NHSO₂CH₃), 2.262.14 (m, 2H, C₃H₂), 1.23-1.03 (m, 2H, NCO₂CH₂CH₂Si(CH₃)₃), 0.05 (s, 9H, NCO₂CH₂CH₂Si(CH₃)₃).

¹³C NMR (100 MHz, CDCl₃, 20° C.): δ 155.8 (NCO₂CH₂CH₂Si(CH₃)₃), 154.8 (NCO₂CH₃), 135.5 (C₄ₐ), 135.4 (C₇ₐ), 127.1 (2C, C₇, C₅), 125.8 (C₆), 121.6 (C₄), 77.8 (C₈ₐ), 66.3 (NCO₂CH₂CH₂Si(CH₃)₃), 52.6 (NCO₂CH₃), 46.0 (C₃ₐ), 45.2 (C₂), 38.7 (NHSO₂CH₃), 28.7 (C₃), 17.9 (NCO₂CH₂CH₂Si(CH₃)₃), −1.5 (NCO₂CH₂CH₂Si(CH₃)₃).

FTIR (thin film) cm⁻¹: 3163 (w), 2955 (m), 1711 (s), 1680 (s), 1160 (s).

HRMS (ESI) (m/z): calculated for C₁₉H₂₉N₃NaO₆SSi [M+Na]⁺: 478.1439. found 478.1430.

[α]_{D}²⁴: −207 (c=0.68, CH₂Cl₂).

TLC (20% acetone in hexanes), Rf: 0.20 (UV, CAM).

### Example 4. Efficient Synthesis of Sulfonylhydrazides

### Synthesis of Sulfonamide (+)-10

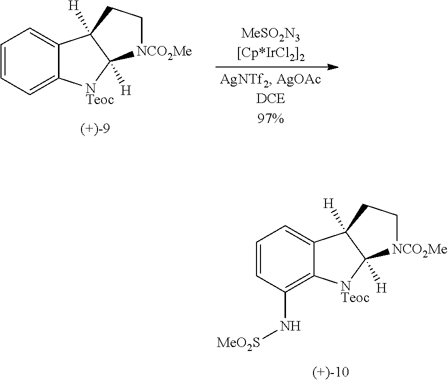

To a suspension of cyclotryptamine (+)-9 (96.6 mg, 266 μmol, 1 equiv), dichloro(pentamethylcyclopentadienyl)iridium (III) dimer ([Cp*IrCl2]₂, 17.0 mg, 21.3 μmol, 8.00 mol %), silver bis(trifluoromethanesulfonyl)imide (33.0 mg, 85.1 μmol, 0.320 equiv) and silver acetate (26.7 mg, 160 μmol, 0.600 equiv) in dichloroethane (0.27 mL) was added methanesulfonyl azide₃ (48.3 mg, 399 μmol, 1.50 equiv) via syringe. The reaction flask was sealed and the reaction was allowed to stir for 20 h. The reaction mixture was filtered through a pad of Celite and the filter cake was rinsed with ethyl acetate (10 mL). The filtrate was concentrated under reduced pressure. The resulting residue was purified by flash column chromatography on silica gel (eluent: gradient, 20→40% ethyl acetate in hexanes) to afford cyclotryptamine sulfonamide (+)-10 (117 mg, 96.5%) as a pale yellow amorphous gum. Structural assignments were made using additional information from gCOSY, HSQC, and HMBC experiments. ₃ Matano, Y.; Ohkubo, H.; Honsho, Y.; Saito, A.; Seki, S.; Imahori, H. Org. Lett. 2013, 15, 932 (the entirety of which is herein explicitly incorporated by reference).

¹H NMR (400 MHz, CDCl₃, 25° C.): δ 9.08 (s, 1H, NHSO₂CH₃), 7.42 (d, J=8.0 Hz, 1H, C₆H), 7.20 (app-t, J=7.8 Hz, 1H, C₅H), 7.10 (app-dt, J=1.1, 7.5 Hz, 1H, C₄H), 6.32 (d, J=5.7 Hz, 1H, C₈ₐH), 4.46-4.27 (m, 2H, C₁₀H₂), 4.06 (app-br-s, 1H, C₃ₐH), 3.65 (app-s, 4H, NCO₂CH₃, C₂Hₐ), 2.79 (app-dd, J=9.6, 18.6 Hz, 1H, C₂H_{b}), 2.68 (s, 3H, NHSO₂CH₃), 2.26-2.14 (m, 2H, C₃H₂), 1.23-1.03 (m, 2H, C₁₁H₂), 0.05 (s, 9H, (C₁₂H₃)₃).

¹³C NMR (100 MHz, CDCl₃, 25° C.): δ 155.8 (C₉), 154.8 (NCO₂CH₃), 135.5 (C₄ₐ), 135.4 (C₇ₐ), 127.1 (2C, C₇, C₅), 125.8 (C₆), 121.6 (C₄), 77.8 (C₈ₐ), 66.3 (C₁₀), 52.6 (NCO₂CH₃), 46.0 (C₃ₐ), 45.2 (C₂), 38.7 (NHSO₂CH₃), 28.7 (C₃), 17.9 (C₁₁), −1.5 (C₁₂).

FTIR (thin film) cm⁻¹: 3163 (w), 2955 (m), 1711 (s), 1680 (s), 1160 (s).

HRMS (ESI) (m/z): calculated for C₁₉H₂₉N₃NaO₆SSi [M+Na]⁺: 478.1439. found 478.1430.

[α]_{D}²⁴: +226 (c=0.61, CH₂Cl₂).

TLC (20% acetone in hexanes), Rf: 0.20 (UV, CAM).

### Synthesis of Sulfonamide (−)-10

To a suspension of cyclotryptamine (−)-9 (278 mg, 767 μmol, 1 equiv), dichloro(pentamethylcyclopentadienyl)iridium (III) dimer ([Cp*IrCl2]2, 48.9 mg, 61.4 μmol, 8.00 mol %), silver bis(trifluoromethanesulfonyl)imide (95.1 mg, 245 μmol, 0.320 equiv) and silver acetate (76.8 mg, 460 μmol, 0.600 equiv) in dichloroethane (0.77 mL) was added methanesulfonyl azide (139 mg, 1.15 mmol, 1.50 equiv) via syringe. The reaction flask was sealed and the reaction was allowed to stir for 20 h. The reaction mixture was filtered through a pad of Celite and the filter cake was rinsed with ethyl acetate (15 mL). The filtrate was concentrated under reduced pressure. The resulting residue was purified by flash column chromatography on silica gel (eluent: gradient, 20→40% ethyl acetate in hexanes) to afford cyclotryptamine sulfonamide (−)-10 (331 mg, 94.7%) as a pale yellow amorphous gum. For full characterization data for cyclotryptamine sulfonamide (−)-10 ([α]_{D}²⁴=−207 (c=0.68, CH₂Cl₂)) see previous procedure in this document.

### Synthesis of Sulfonylhydrazide(−)-11

Cyclotryptamine sulfonamide (−)-10 (2.02 g, 4.43 mmol, 1 equiv) was azeotropically dried from anhydrous benzene (3×5 mL) and the residue was dissolved in tetrahydrofuran (44 mL). The solution was cooled to 0° C. and sodium hydride (60% in mineral oil, 230 mg, 5.76 mmol, 1.30 equiv) was added in one portion. The ice-water bath was removed and after 30 min, O-(diphenylphosphinyl)hydroxylamine (1.34 g, 5.76 mmol, 1.30 equiv) was added in one portion. After 1 h, the reaction mixture was diluted with ethyl acetate (30 mL), washed with mixture of saturated aqueous sodium bicarbonate and water (10:1 v/v, 25 mL) and the aqueous layer was extracted with ethyl acetate (3×20 mL). The combined organic extracts were dried over anhydrous sodium sulfate, were filtered, and were concentrated under reduced pressure. The resulting residue was purified by flash column chromatography on silica gel (eluent: 25→50% ethyl acetate in hexanes) to afford hydrazidocyclotryptamine (−)-11 (1.70 g, 81.5%) as an orange amorphous gum.

¹H NMR (400 MHz, CDCl₃, 25° C.): δ 7.31 (app-p, J=3.7 Hz, 1H, C₅H), 7.20-7.14 (m, 2H, C₄H, C₆H), 6.27 (br-d, J=5.7 Hz, 1H, C₈ₐH), 4.50 (br-s, 2H, NH₂), 4.29 (t, J=8.9 Hz, 2H, C₁₀H₂), 4.02 (t, J=6.0 Hz, 1H, C₃ₐH), 3.66 (s, 3H, NCO₂CH₃), 3.55 (br-s, 1H, C₂Hₐ), 3.04 (s, 3H, SO₂CH₃), 2.79 (td, J=6.1, 11.0 Hz, 1H, C₂H_{b}), 2.21-2.01 (m, 2H, C₃H₂), 1.22-1.02 (m, 2H, C₁₁H₂), 0.03 (s, 9H, (C₁₂H₃)₃).

¹³C NMR (100 MHz, CDCl₃, 25° C.): δ 154.9 (C₉), 154.6 (NCO₂CH₃), 140.0 (C₇ₐ), 136.7 (C₄ₐ), 133.3 (C₇), 126.8 (C₅), 123.9 (2C, C₄, C₆), 78.1 (C₈ₐ), 65.2 (C₁₀), 52.5 (NCO₂CH₃), 46.1 (br, C₃ₐ), 44.8 (C₂), 37.7 (SO₂CH₃), 29.3 (C₃), 17.9 (C₁₁), −1.5 (C₁₂).

FTIR (thin film) cm⁻¹: 3366 (m), 2954 (m), 1700 (s), 1653 (w), 1559 (w), 1457 (s), 1337 (m).

HRMS (ESI) (m/z): calculated for C₁₉H₃₀N₄NaO₆SSi [M+Na]⁺: 493.1548. found 493.1519.

[α]_{D}²⁴: −119 (c=0.49, CH₂Cl₂).

TLC (50% ethyl acetate in hexanes), Rf: 0.18 (UV, CAM).

### Example 5. Diazene Formation Reactions

Diazene Formation with Hydrazidobenzene

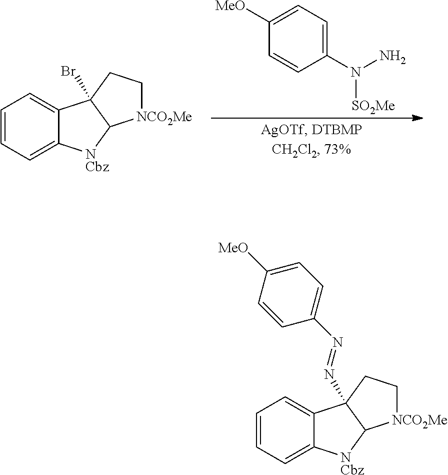

A sample of silver trifluoromethanesulfonate (20.0 mg, 78.0 μmol, 2.00 equiv) was added to a solution of bromocyclotryptamine (16.8 mg, 39.0 μmol, 1 equiv), N-(4-methoxyphenyl)methanesulfonohydrazide (12.8 mg, 59.0 μmol, 1.50 equiv), and 2,6-di-tert-butyl-4-methylpyridine (20.1 mg, 98.0 μmol, 2.50 equiv) in dichloromethane (400 μL) at 22° C. After 1.5 h, the off-white suspension was filtered through a pad of Celite. The filter cake was washed with ethyl acetate (5 mL) and the filtrate was concentrated under reduced pressure. The resulting residue was purified by flash column chromatography on silica gel (eluent: 20%→30% ethyl acetate in hexanes) to afford diazene (13.9 mg, 73.3%) as a bright yellow oil.

Diazene Formation with Hydrazidocyclotryptamine

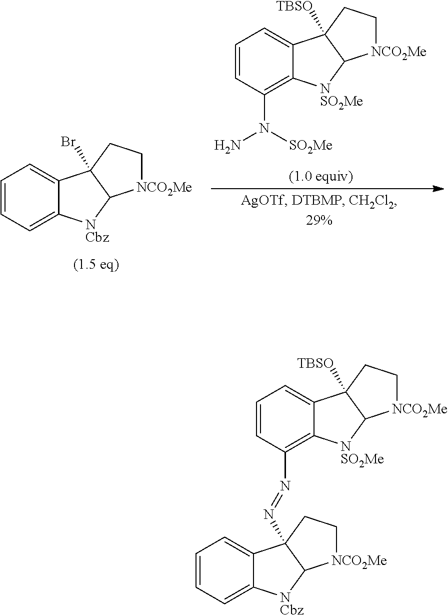

A sample of silver trifluoromethanesulfonate (13.9 mg, 54.0 μmol, 2.00 equiv) was added to a solution of bromocyclotryptamine (15.1 mg, 35.0 μmol, 1.30 equiv), cyclotryptamine methanesulfonohydrazide (14.6 mg, 27.0 μmol, 1 equiv), and 2,6-di-tert-butyl-4-methylpyridine (14.0 mg, 68.0 μmol, 2.50 equiv) in dichloromethane (350 μL) at 22° C. After 1.5 h, the off-white suspension was filtered through a pad of Celite. The filter cake was washed with ethyl acetate (5 mL) and the filtrate was concentrated under reduced pressure. The resulting residue was purified by flash column chromatography on silica gel (eluent: 15%→80% ethyl acetate in hexanes). A fraction of material was then repurified by HPLC to afford cyclotryptamine diazene (6.20 mg, 28.5%) as a bright yellow solid.

As a result of the slow conformational equilibration at ambient temperature, NMR spectra were collected at elevated temperature.

Diazene Formation with Hydrazidocyclotryptamine

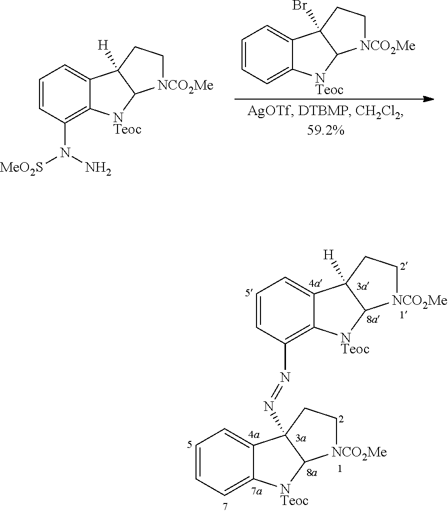

A sample of silver trifluoromethanesulfonate (673 mg, 2.62 mmol, 2.00 equiv) was added to a solution of bromocyclotryptamine (578 mg, 1.31 mmol, 1 equiv), cyclotryptamine methanesulfonohydrazide (802 mg, 1.70 mmol, 1.30 equiv), and 2,6-di-tert-butyl-4-methylpyridine (674 mg, 3.28 mmol, 2.50 equiv) in dichloromethane (13.0 mL) at 22° C. After 1 h, the off-white suspension was filtered through a pad of Celite. The filter cake was washed with ethyl acetate (20 mL) and the filtrate was concentrated under reduced pressure. The resulting residue was purified by flash column chromatography on silica gel (eluent: 25%→35% ethyl acetate in hexanes) to yield the cyclotryptamine dimer (582 mg, 59.2%) as a bright yellow amorphous gum.

¹H NMR (400 MHz, CDCl₃, 20° C.): δ 7.80 (d, J=8.1 Hz, 1H, C₇H), 7.337.20 (m, 3H, C₆H, C₄H, C_{4′}H), 7.17 (d, J=7.7 Hz, 1H, C_{6′}H), 7.157.08 (m, 1H, C_{5′}H), 7.03 (app-t, J=7.5 Hz, 1H, C₅H), 6.89 (s, 1H, C₈ₐH), 6.41 (br-s, 1H, C_{8a′}H), 4.434.31 (m, 2H, N₈CO₂CH₂CH₂Si(CH₃)₃), 4.314.23 (m, 1H, C_{3a′}H), 4.143.96 (m, 3H, C₂Hₐ, N_{8′}CO₂CH₂CH₂Si(CH₃)₃), 3.893.67 (m, 7H, C_{2′}Hₐ, N₁CO₂CH₃, N₁CO₂CH₃), 3.10 (td, J=5.2, 11.7 Hz, 1H, C₂H_{b}), 2.93 (td, J=6.9, 10.8 Hz, 1H, C_{2′}H_{b}), 2.622.46 (m, 1H, C₃Hₐ), 2.37 (dd, J=5.1, 12.6 Hz, 1H, C₃H_{b}), 2.282.13 (m, 2H, Cy₃H₂), 1.15 (dd, J=6.8, 10.8 Hz, 2H, N₈CO₂CH₂CH₂Si(CH₃)₃), 0.96 (br-s, 2H, N_{8′}CO₂CH₂CH₂Si(CH₃)₃), 0.06 (s, 9H, N₈CO₂CH₂CH₂Si(CH₃)₃), −0.02 (s, 9H, N_{8′}CO₂CH₂CH₂Si(CH₃)₃).

¹³C NMR (100 MHz, CDCl₃, 20° C.): δ 155.4 (N₁CO₂CH₃ or N₁CO₂CH₃), 155.3 (N₁CO₂CH₃ or N₁CO₂CH₃), 154.9 (N₈CO₂CH₂CH₂Si(CH₃)₃ or N_{8′}CO₂CH₂CH₂Si(CH₃)₃), 153.8 (N₈CO₂CH₂CH₂Si(CH₃)₃ or N_{8′}CO₂CH₂Si(CH₃)₃), 143.5 (C₇ₐ), 141.6 (C_{7′}), 139.7 (C_{7a′}), 135.7 (C_{4a′}), 130.0 (C₆), 129.2 (C₄ₐ), 125.7 (C_{5′}), 125.4 (C₄ or C_{4′}), 125.3 (C₄ or C_{4′}), 123.5 (C₅), 117.1 (C_{6′}), 116.2 (C₇), 88.9 (C₃ₐ), 79.6 (C₈ₐ), 79.1 (C_{8a′}), 64.8 (C_{3a′}), 64.5 (N₈CO₂CH₂CH₂Si(CH₃)₃), 52.8 (N₁CO₂CH₃ or N₁CO₂CH₃), 52.7 (N₁CO₂CH₃ or N₁CO₂CH₃), 46.4 (N_{8′}CO₂CH₂CH₂Si(CH₃)₃), 46.1 (C₂), 45.2 (C_{2′}), 35.9 (C₃), 29.6 (C_{3′}), 17.9 (2C, N₈CO₂CH₂CH₂Si(CH₃)₃, N_{8′}CO₂CH₂CH₂Si(CH₃)₃), −1.4 (N₈CO₂CH₂CH₂Si(CH₃)₃ or N_{8′}CO₂CH₂CH₂Si(CH₃)₃), −1.5 (N₈CO₂CH₂CH₂Si(CH₃)₃ or N_{8′}CO₂CH₂CH₂Si(CH₃)₃).

FTIR (thin film) cm⁻¹: 2954 (m), 1707 (s), 1602 (w), 1397 (m), 1259 (m).

HRMS (ESI) (m/z): calculated for C₃₆H₅₀N₆NaO₈Si₂ [M+Na]⁺: 773.3121. found 773.3115.

[α]_{D}²⁴: −86 (c=0.61, CH₂Cl₂).

TLC (50% ethyl acetate in hexanes), Rf: 0.32 (UV, CAM).

### Example 6. Representative Csp2-Csp3 Bond Formation

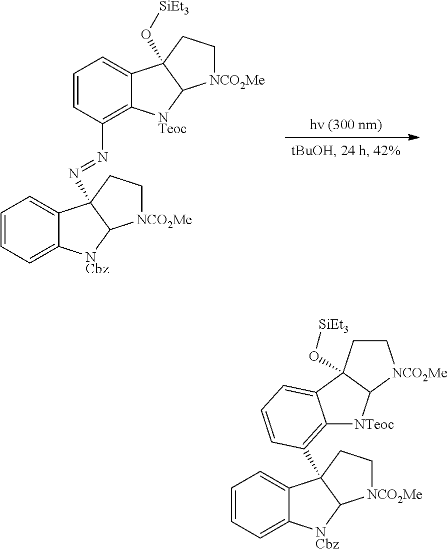

A solution of cyclotryptamine diazene dimer (6.00 mg, 7.00 μmol, 1 equiv) in degassed (N₂ stream, 5 min) tert-butanol (700 L) was irradiated in a Rayonet photoreactor equipped with 16 radially distributed (r=12.7 cm) 25 W lamps (A=300 nm) at 25° C. After 24 h, the reaction mixture was concentrated under reduced pressure. The resulting residue was purified by flash column chromatography on silica gel (eluent: 30% ethyl acetate in hexanes) to afford coupled cyclotryptamine dimer (2.50 mg, 42.4%) as an off-white solid.

### Example 7. Representative Examples of CH-Amination

In a diazene-containing intermediate

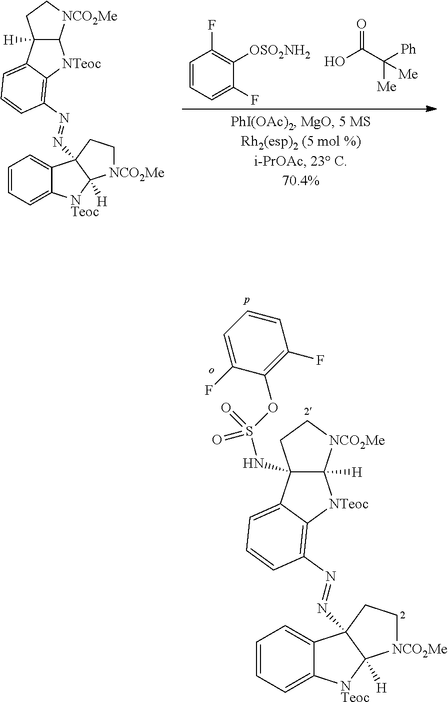

A round bottom flask was charged with 5 Å molecular sieves (13.4 mg, 200 mg/mmol of diazene starting material), magnesium oxide (10.8 mg, 268 μmol, 4.00 equiv) and flame-dried under vacuum for 5 min. The reaction vessel was allowed to cool to 22° C. and back filled with argon. Solid 2,6-difluorophenyl sulfamate₄ (17.8 mg, 87.0 mol, 1.30 equiv), 2-methyl-2-phenylpropionic acid (5.60 mg, 34.0 μmol, 0.500 equiv), and Rh₂(esp)₂ (0.500 mg, 0.70 μmol, 0.0100 equiv) were added sequentially. A solution of cyclotryptamine diazene dimer (50.0 mg, 67.0 μmol, 1 equiv) in isopropyl acetate (130 L) was added via syringe at 22° C. and the mixture was allowed to stir. After 5 min, (diacetoxyiodo)benzene (43.2 mg, 134 μmol, 2.00 equiv) was added and the green heterogeneous mixture was agitated by vigorous stirring at 22° C. After 14 h, another portion of Rh₂(esp)₂ (1.00 mg, 1.40 μmol, 0.0200 equiv) was added. After 4 h, the reaction mixture was filtered through a pad of Celite and the filter cake was rinsed with ethyl acetate (5 mL). The filtrate was concentrated under reduced pressure. The resulting residue was purified by flash column chromatography on silica gel (eluent: gradient, 35→50% ethyl acetate in hexanes) to afford cyclotryptamine dimer sulfamate ester (45.2 mg, 70.4%) as a bright yellow amorphous gum. Structural assignments were made using additional information from gCOSY, HSQC, and HMBC experiments. ₄ J. L. Roizen. D. N. Zalatan and J. Du Bois, Angew. Chem. Int. Ed, 2013, Early View, DOI: 10.1002/anie.201304238 (the entirety of which is herein explicitly incorporated by reference).

¹H NMR (400 MHz, CDCl₃, 20° C.): δ 7.77 (d, J=8.1 Hz, 1H, C₇H), 7.48 (d J=6.6 Hz, 1H, C_{4′}H), 7.337.27 (m, 2H, C₆H, C_{6′}H), 7.257.13 (m, 3H, C_{4′}H, C_{5′}H, CₚH), 7.01 (app-t, J=7.2 Hz, 1H, C₅H), 6.96 (t, J=8.1 Hz, 2H, CₘH), 6.85 (br-s, 1H, C₈ₐH), 6.58 (br-s, 1H, C_{8a′}H). 6.16 (br-s, 1H, NH), 4.394.27 (m, 2H, N₈CO₂CH₂CH₂Si(CH₃)₃ or N_{8′}CO₂CH₂CH₂Si(CH₃)₃), 4.274.19 (m, 1H, N₈CO₂CHₐCH₂Si(CH₃)₃ or N_{8′}CO₂CHₐCH₂Si(CH₃)₃), 4.133.96 (m, 2H, N₈CO₂CH_{b}CH₂Si(CH₃)₃ or N_{8′}CO₂CH_{b}CH₂Si(CH₃)₃, C₂Hₐ), 3.833.68 (m, 7H, C_{2′}Hₐ, N₁CO₂CH₃, N₁CO₂CH₃), 3.07 (td, J=5.2, 11.7 Hz, 1H, C₂H_{b}), 2.90 (br-s, 1H, C_{3′}Hₐ), 2.81 (br-s, 1H, C_{2′}H_{b}), 2.632.45 (m, 2H, C₃Hₐ, C_{3′}H_{b}), 2.35 (dd, J=5.1, 12.5 Hz, 1H, C₃H_{b}), 1.171.07 (m, 2H, N₈CO₂CH₂CH₂Si(CH₃)₃ or N_{8′}CO₂CH₂CH₂Si(CH₃)₃), 1.030.80 (m, 2H, N₈CO₂CH₂CH₂Si(CH₃)₃ or N_{8′}CO₂CH₂CH₂Si(CH₃)₃), 0.05 (s, 9H, N₈CO₂CH₂CH₂Si(CH₃)₃ or N_{8′}CO₂CH₂CH₂Si(CH₃)₃), −0.03 (s, 9H, N₈CO₂CH₂CH₂Si(CH₃)₃ or N_{8′}CO₂CH₂CH₂Si(CH₃)₃).

¹³C NMR (100 MHz, CDCl₃, 20° C.): δ 156.0 (dd, J=3.3, 253.7 Hz, Cₒ), 155.4 (2C, N₁CO₂CH₃, N₁CO₂CH₃), 154.6 (N₈CO₂CH₂CH₂Si(CH₃)₃ or N_{8′}CO₂CH₂CH₂Si(CH₃)₃), 153.7 (N₈CO₂CH₂CH₂Si(CH₃)₃ or N_{8′}CO₂CH₂CH₂Si(CH₃)₃), 143.5 (C₇ₐ), 141.8 (C_{7′}), 140.3 (C_{7a′}), 133.1 (C_{4a′}), 130.0 (C₆), 129.0 (C₄ₐ), 127.8 (t, J=9.2 Hz, Cₚ), 126.9 (t, J=15.6 Hz, Cᵢ), 126.2 (C_{5′}), 125.5 (C₄), 125.4 (C_{4′}), 123.5 (C₅), 119.7 (C_{6′}), 116.3 (C₇), 112.7 (dd, J=4.4, 17.7 Hz, Cₘ), 88.9 (C₃ₐ), 81.6 (C_{8a′}), 79.7 (C₈ₐ), 71.6 (C_{3a′}), 65.2 (N₈CO₂CH₂CH₂Si(CH₃)₃ or N_{8′}CO₂CH₂CH₂Si(CH₃)₃), 64.5 (N₈CO₂CH₂CH₂Si(CH₃)₃ or N_{8′}CO₂CH₂CH₂Si(CH₃)₃), 52.8 (2C, N₁CO₂CH₃, N₁CO₂CH₃), 46.1 (C₂), 45.0 (C_{2′}), 35.6 (C₃), 33.4 (C_{3′}), 17.9 (N₈CO₂CH₂CH₂Si(CH₃)₃ or N_{8′}CO₂CH₂CH₂Si(CH₃)₃), 17.7 (N₈CO₂CH₂CH₂Si(CH₃)₃ or N_{8′}CO₂CH₂CH₂Si(CH₃)₃), −1.4 (N₈CO₂CH₂CH₂Si(CH₃)₃ or N_{8′}CO₂CH₂CH₂Si(CH₃)₃), −1.5 (N₈CO₂CH₂CH₂Si(CH₃)₃ or N_{8′}CO₂CH₂CH₂Si(CH₃)₃).

FTIR (thin film) cm⁻¹: 3162 (s), 2955 (s), 1717 (s), 1457 (m), 862 (w), 733 (w).

HRMS (ESI) (m/z): calculated for C₄₂H₅₃F₂N₇NaO₁₁SSi₂ [M+Na]⁺: 980.2923. found 980.2917.

[α]_{D}²⁴: 76 (c=0.72, CH₂Cl₂).

TLC (50% ethyl acetate in hexanes), Rf: 0.28 (UV, CAM).

In a Dimer with a Csp2-Csp3 Bond in Place

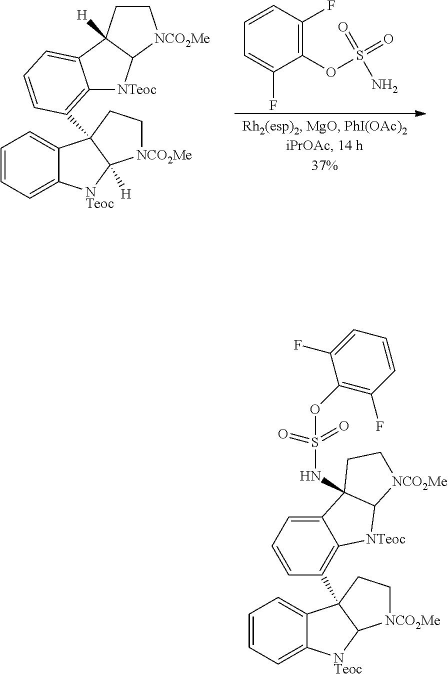

A round bottom flask was charged with 5 Å molecular sieves (39.0 mg, 200 mg/mmol of cyclotryptamine dimer starting material), magnesium oxide (31.4 mg, 780 μmol, 4.00 equiv) and flame-dried under vacuum for 5 min. The reaction vessel was allowed to cool to 22° C. and back filled with argon. Solid 2,6-difluorophenyl sulfamate₅ (59.8 mg, 293 μmol, 1.30 equiv), 2-methyl-2-phenylpropionic acid (16.1 mg, 98.0 μmol, 0.500 equiv), and Rh₂(esp)₂ (1.50 mg, 2.00 μmol, 0.0100 equiv) were added sequentially. A solution of cyclotryptamine diazene dimer (141 mg, 195 μmol, 1 equiv) in isopropyl acetate (390 μL) was added via syringe at 22° C. and the mixture was allowed to stir. After 5 min, (diacetoxyiodo)benzene (126 mg, 390 μmol, 2.00 equiv) was added and the green heterogeneous mixture was agitated by vigorous stirring at 22° C. After 3 h, another portion of Rh₂(esp)₂ (1.50 mg, 2.00 μmol, 0.0100 equiv) was added. After 22 h, the reaction mixture was filtered through a pad of Celite and the filter cake was rinsed with ethyl acetate (5 mL). The filtrate was concentrated under reduced pressure. The resulting residue was purified by flash column chromatography on silica gel (eluent: gradient, 8→20% acetone in hexanes) to afford cyclotryptamine dimer sulfamate ester (66.7 mg, 36.8%) as a bright yellow amorphous gum. ₅ J. L. Roizen, D. N. Zalatan and J. Du Bois Angew. Chem. Int. Ed, 2013, Early View, DOI: 10.1002/anie.201304238 (the entirety of which is herein explicitly incorporated by reference).

### Example 8. Representative Procedure for Silyl Group Deprotection

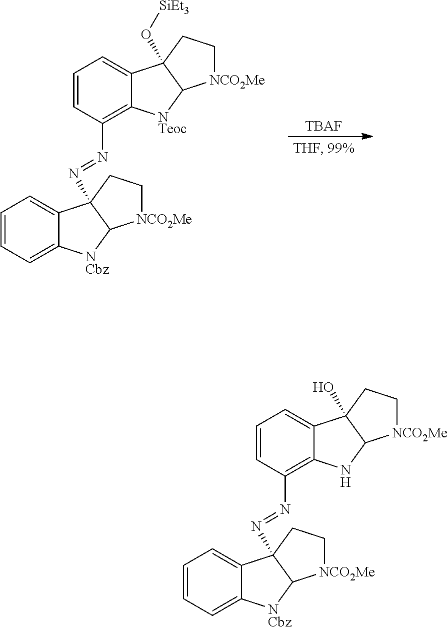

Tetrabutylammonium fluoride (1M in THF, 60.0 μL, 60.0 μmol, 10.0 equiv) was added dropwise to a solution of cyclotryptamine dimer (5.60 mg, 6.00 μmol, 1 equiv) in tetrahydrofuran (100 μL) at 22° C. After 1.5 h, the reaction mixture was concentrated under reduced pressure. The resulting residue was purified by flash column chromatography on silica gel (eluent: 50% ethyl acetate in hexanes) to afford deprotected cyclotryptamine dimer (4.40 mg, >99.9%) as a bright yellow oil.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

We claim:

1. A method of preparing compounds of Formula (I), or a pharmaceutically acceptable salt, tautomer or stereoisomer thereof, comprising reacting a compound of Formula (II), and thereby extruding dinitrogen to provide a compound of Formula (I): 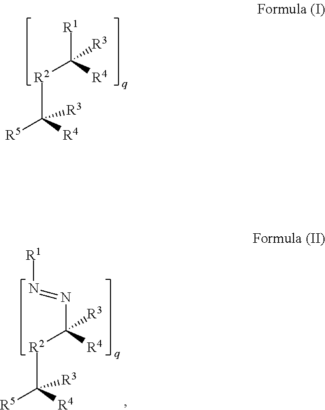
R¹ is tertiary alkyl, alkenyl, aryl, heteroaryl, carbocyclyl, or heterocyclyl or at least one moiety of structure: 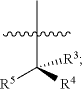
and
R², R³, R⁴, and R⁵ are each occurrence, each independently selected from tertiary alkyl, aryl, heteroaryl, carbocyclyl, or heterocyclic, wherein any two of R³, R⁴, and R⁵ taken together with the carbon atoms to which they are attached form a C₅₋₁₄ membered saturated, unsaturated, or aromatic carbocyclic ring or a C₅₋₁₄ membered saturated, unsaturated, or aromatic heterocyclic ring; and wherein any tertiary alkyl, aryl, heteroaryl, carbocyclyl, or heterocyclyl, C₅₋₁₄ membered saturated, unsaturated, or aromatic carbocyclic ring or a C₅₋₁₄ membered saturated, unsaturated, or aromatic heterocyclic ring can be further substituted with one or more halogen, alkyl, heteroaryl, carbocyclyl, heterocyclyl, C₃₋₁₄ membered saturated, unsaturated, or aromatic carbocyclic, or C₃₋₁₄ membered saturated, unsaturated, or aromatic heterocyclic rings; and
q is an integer from 0-8.

2. The method of claim 1, wherein the compound of Formula (I) is: 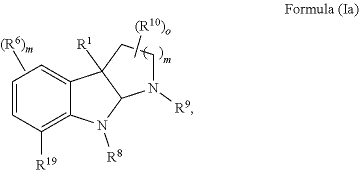
wherein
R¹ is tertiary alkyl, alkenyl, aryl, heteroaryl, carbocyclyl, heterocyclyl, 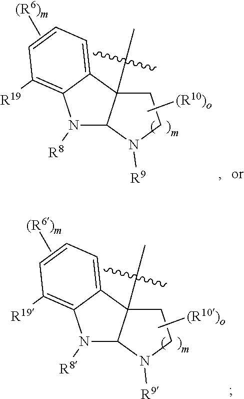
R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R⁹, R^{8′}, and R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)R¹³, —OH, —OR¹¹, —OC(═O)R¹¹, —NR¹¹R¹², aryl, heteroaryl, carbocyclyl, or heterocyclyl, wherein two R¹⁰ or two R¹⁰ groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R¹⁹ and R^{19′} are each independently selected from H, tertiary alkyl, alkenyl, aryl, heteroaryl, carbocyclyl, heterocyclyl, 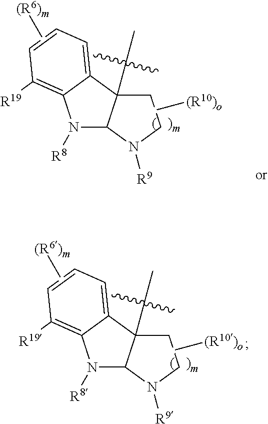
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

3. The method of claim 2, wherein R¹ is comprised of 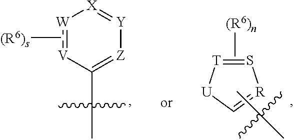
wherein
V, W, X, Y, and Z are each independently selected from —CH or N;
R, S, and T are each independently selected from —CH or N; and
U is O, S, or NR¹¹; wherein
R¹¹ is independently selected from H, C₁-C₁₂ alkyl, C₂-C₁₂ alkenyl, C₂-C₁₂ alkynyl, aryl, heteroaryl, carbocyclyl, or heterocyclyl; and
n is an integer from 0-4; and
s is an integer from 0-5.

4. The method of claim 2, wherein the compound of Formula (I) is: 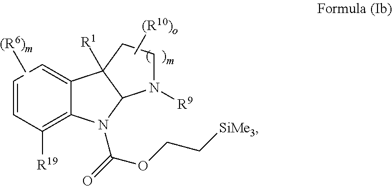
wherein
R¹⁹ is H, tertiary alkyl, alkenyl, aryl, heteroaryl, carbocyclyl, heterocyclyl, 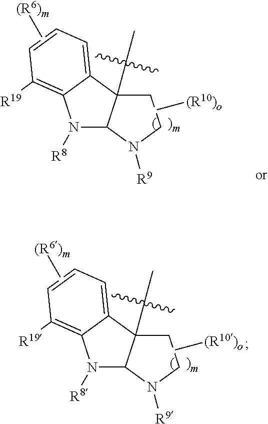
and
R⁸ and R^{8′} is —C(═O)O(CH₂)₂SiMe₃.

5. The method of claim 1, wherein the compound of Formula (II) is: 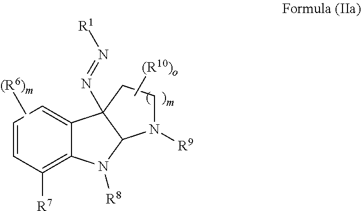
wherein
R¹ is tertiary alkyl, alkenyl, aryl, heteroaryl, carbocyclyl, heterocyclyl, 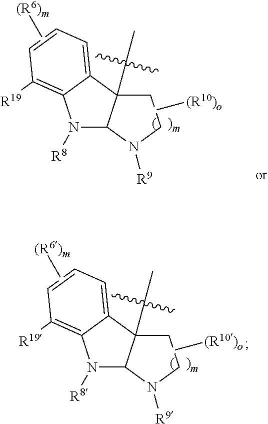
R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁷ and R^{7′} are each independently selected from H, —N₃, —N(R¹⁵)NH₂, —NHR¹⁵, 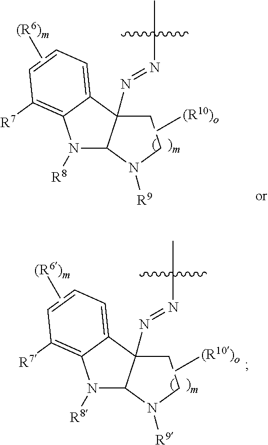
R⁸, R^{8′}, R⁹, and, R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R^{1′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R¹⁹ and R^{19′} are each independently H;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

6. The method of claim 5, wherein R¹ is, 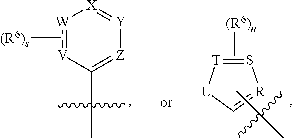
wherein
V, W, X, Y, and Z are each independently selected from —CH or N;
R, S, and T are each independently selected from —CH or N;
U is O, S, or NR¹¹; wherein
R¹¹ is independently selected from H, C₁-C₁₂ alkyl, C₂-C₁₂ alkenyl, C₂-C₁₂ alkynyl, —Si(alkyl)₃, —Si(alkyl)₂aryl, Si(aryl)₂alkyl, aryl, heteroaryl, carbocyclyl, or heterocyclyl, and wherein R¹¹ and R¹² taken together with the carbon atoms to which they are attached form an aryl, heteroaryl, carbocyclic, or heterocyclic ring; and
n is an integer from 0-4; and
s is an integer from 0-5.

7. The method of claim 1, wherein the compound of Formula (II) is prepared by reacting a compound of Formula (III), and a compound of Formula (IV): 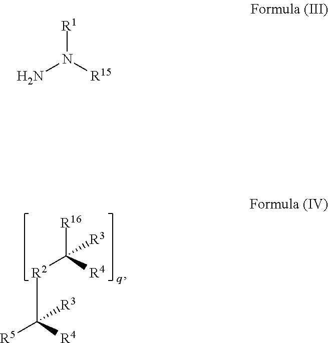
wherein
R¹ is alkenyl, aryl, or heteroaryl; and
R², R³, R⁴, and R⁵ are each occurrence, each independently selected from alkyl, aryl, heteroaryl, carbocyclyl, or heterocyclic, wherein any two of R³, R⁴, and R⁵ taken together with the carbon atoms to which they are attached form a C₅₋₁₄ membered saturated, unsaturated, or aromatic carbocyclic ring or a C₅₋₁₄ membered saturated, unsaturated, or aromatic heterocyclic ring; and wherein any tertiary alkyl, aryl, heteroaryl, carbocyclyl, or heterocyclyl, C₅₋₁₄ membered saturated, unsaturated, or aromatic carbocyclic ring or a C₅₋₁₄ membered saturated, unsaturated, or aromatic heterocyclic ring can be further substituted with one or more halogen, alkyl, heteroaryl, carbocyclyl, heterocyclyl, C₃₋₁₄ membered saturated, unsaturated, or aromatic carbocyclic, or C₃₋₁₄ membered saturated, unsaturated, or aromatic heterocyclic rings;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR¹¹, —P(═O)R¹³R¹⁴, or, —P(═O)NR¹¹R¹²;
R¹⁶ is I, Br, Cl, —OH, —OSO₂CF₃, —OS(O)₂R¹³, —OP(═O)R¹³R¹⁴, —OC(═NR¹¹)R¹², —OC(═NR¹¹)CCl₃, —OR¹¹, or —N₂⁺X⁻, wherein X⁻ is halogen;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2;
q is an integer of from 0-8; and
r is an integer from 1 to 4.

8. The method of claim 7, wherein the compound of Formula (III) is: 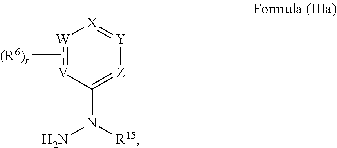
wherein
V, W, X, Y, and Z are each independently selected from —CH or N; and
r is an integer from 0 to 5.

9. The method of claim 7, wherein the compound of Formula (III) is: 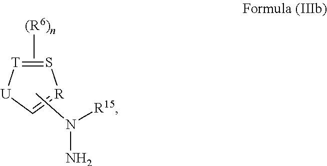
wherein
R, S, and T are each independently selected from —CH or N; and
U is O, S, or NR¹¹, wherein R¹¹ is independently selected from H, C₁-C₁₂ alkyl, C₂-C₁₂ alkenyl, C₂-C₁₂ alkynyl, aryl, heteroaryl, carbocyclyl, or heterocyclyl; and
n is an integer from 0 to 4.

10. The method of claim 7, wherein the compound of Formula (III) is: 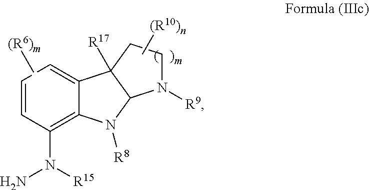
wherein
R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R¹⁷ is H, —OH, —OR¹¹, —NR¹¹R¹², aryl, heteroaryl, carbocyclyl, heterocyclyl, 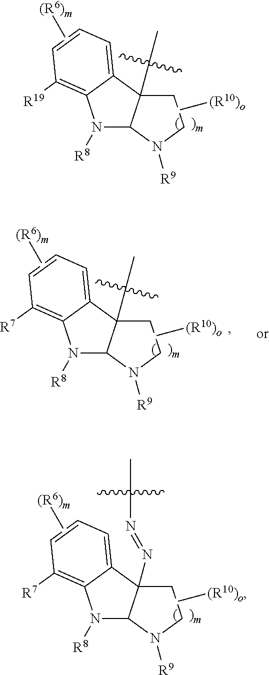
R⁷ and R^{7′} are each independently selected from H, —N₃, —N(R¹⁵)NH₂, —NHR⁵, 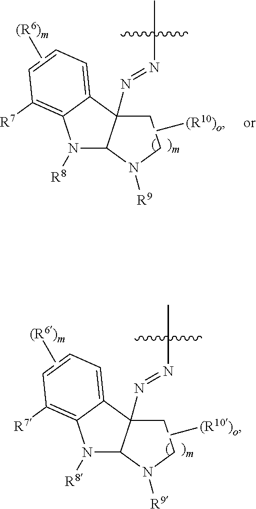
and wherein
R¹⁹ and R^{19′} are each independently selected from H, tertiary alkyl, alkenyl, aryl, heteroaryl, carbocyclyl, heterocyclyl 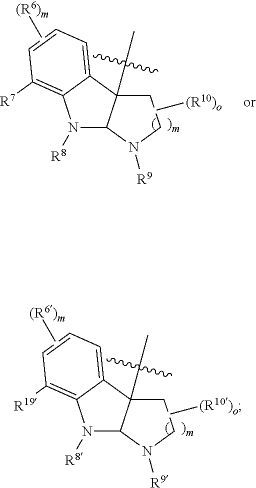
R⁸, R^{8′}, R⁹, and, R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R¹⁰ or two R¹⁰ groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

11. The method of claim 7, wherein R¹⁵ is —S(═O)ₚR¹³, wherein p is 2, and R¹³ is C₁-C₁₂ alkyl.

12. The method of claim 11, wherein the compound of Formula (II) is formed in one synthetic step.

13. The method of claim 7, wherein the compound of Formula (IV) is: 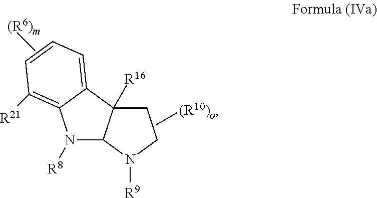
wherein
R¹⁶ is I, Br, Cl, —OH, —OSO₂CF₃, —OS(O)₂R¹³, —OP(═O)R¹³R¹⁴, —OC(═NR¹¹)R¹², —OC(═NR¹¹)CCl₃, —OR¹¹, or —N₂⁺X⁻, wherein X⁺ is halogen;
R²¹ is H, halogen, alkyl, alkenyl, alkynyl, aryl, heteroaryl, carbocyclyl, heterocyclyl, —N₃, 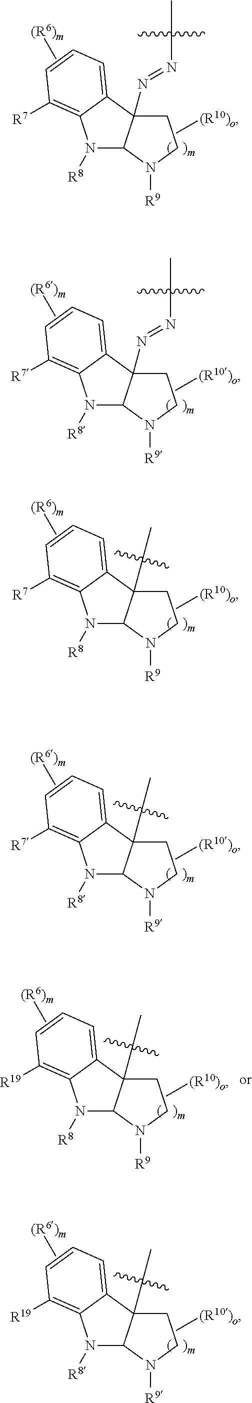
wherein
R⁷ and R^{7′} are each independently selected from H, —N₃, —N(R¹⁵)NH₂, —NHR¹⁵, 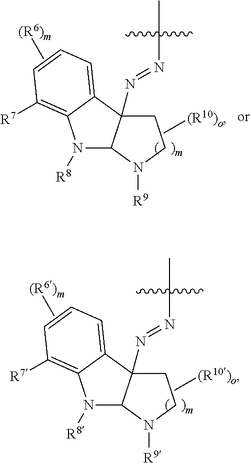
and wherein
R¹⁹ and R^{19′} are each independently selected from H, tertiary alkyl, alkenyl, aryl, heteroaryl, carbocyclyl, heterocyclyl, 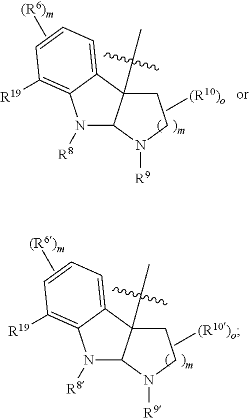
R⁶ and R^{6′} are each independently selected from halogen, —OH, —OR¹¹, —OC(═O)R¹¹, —NR¹¹R¹², —S(═O)ₚR¹¹, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R^{8′}, R⁹, and, R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹¹, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

14. The method of claim 10, wherein the compound of Formula (IV) is: 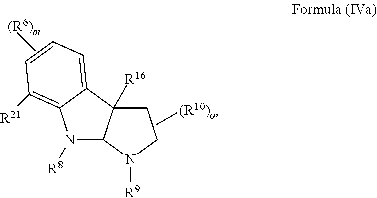
wherein
R¹⁶ is I, Br, Cl, —OH, —OSO₂CF₃, —OS(O)₂R¹³, —OP(═O)R¹³R¹⁴, —OC(═NR¹¹)R¹², —OC(═NR¹¹)CCl₃, —OR¹¹, or —N₂⁺X⁻, wherein X⁻ is halogen;
R²¹ is H, halogen, alkyl, alkenyl, alkynyl, aryl, heteroaryl, carbocyclyl, heterocyclyl, —N₃, 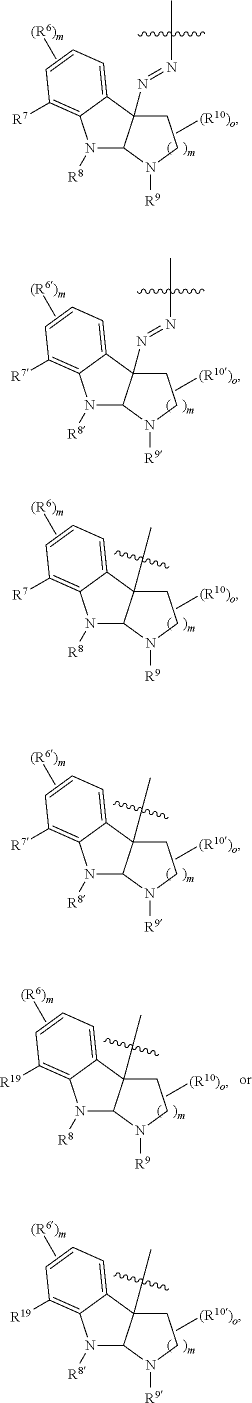
wherein
R⁷ and R^{7′} are each independently selected from H, —N₃, —N(R¹⁵)NH₂, —NHR¹⁵, 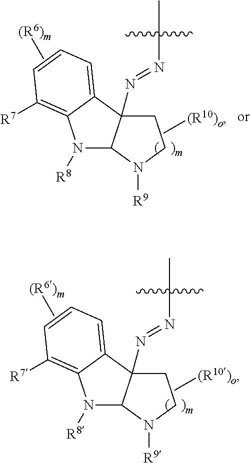
and wherein
R¹⁹ and R^{19′} are each independently selected from H, tertiary alkyl, alkenyl, aryl, heteroaryl, carbocyclyl, heterocyclyl 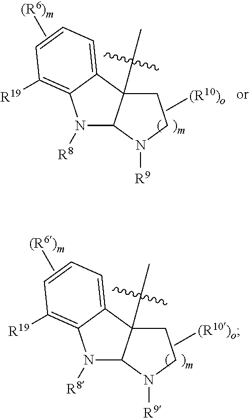
R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R^{8′}, R⁹, and, R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹¹, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

15. The method of claim 1, wherein the compound of Formula (II) is prepared by the extrusion of sulfur dioxide from a compound of Formula (V): 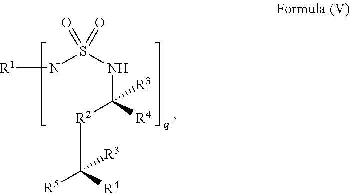
wherein
R¹ is tertiary alkyl, alkenyl, aryl, heteroaryl, carbocyclyl, or heterocyclyl or at least one moiety of structure: 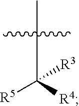
and
R², R³, R⁴, and R⁵ are each occurrence, each independently selected from alkyl, aryl, heteroaryl, carbocyclyl, or heterocyclic, wherein any two of R³, R⁴, and R⁵ taken together with the carbon atoms to which they are attached form a C₅₋₁₄ membered saturated, unsaturated, or aromatic carbocyclic ring or a C₅₋₁₄ membered saturated, unsaturated, or aromatic heterocyclic ring; and wherein any tertiary alkyl, aryl, heteroaryl, carbocyclyl, or heterocyclyl, C₅₋₁₄ membered saturated, unsaturated, or aromatic carbocyclic ring or a C₅₋₁₄ membered saturated, unsaturated, or aromatic heterocyclic ring can be further substituted with one or more halogen, alkyl, heteroaryl, carbocyclyl, heterocyclyl, C₃₋₁₄ membered saturated, unsaturated, or aromatic carbocyclic, or C₃₋₁₄ membered saturated, unsaturated, or aromatic heterocyclic rings; and
q is an integer of from 0-8.

16. The method of claim 15, wherein the compound of Formula (V) is: 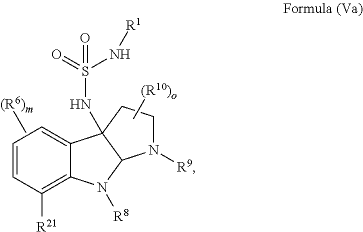
wherein
R¹ is tertiary alkyl, alkenyl, aryl, heteroaryl, carbocyclyl, heterocyclyl, 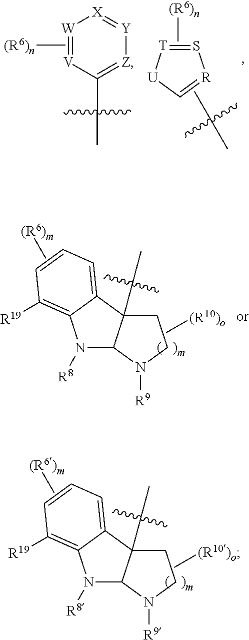
V, W, X, Y, and Z are each independently selected from —CH or N;
R, S, and T are each independently selected from —CH or N;
U is O, S, or NR¹¹;
R²¹ is H, halogen, alkyl, alkenyl, alkynyl, aryl, heteroaryl, carbocyclyl, heterocyclyl, —N₃, 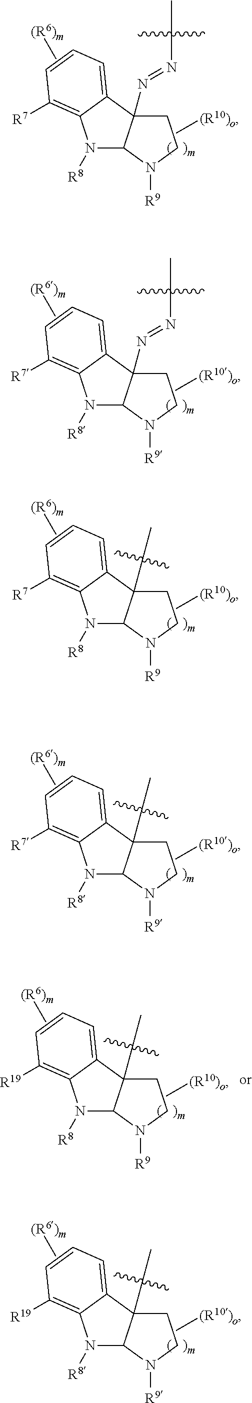
wherein
R⁷ and R^{7′} are each independently selected from H, —N₃, —N(R¹⁵)NH₂, —NHR¹⁵, 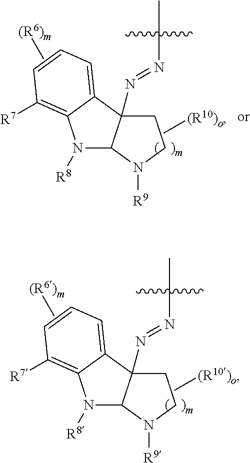
and wherein
R¹⁹ and R^{19′} are each independently selected from H, tertiary alkyl, alkenyl, aryl, heteroaryl, carbocyclyl, heterocyclyl 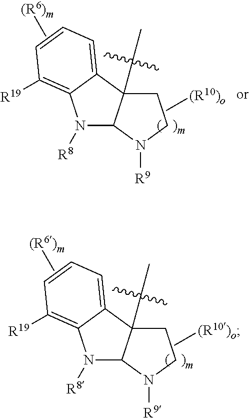
R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R^{8′}, R⁹, and R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², —SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

17. The method of claim 15, wherein the compound of Formula (V) is prepared by the following steps:
a. reacting a compound of Formula (VI), and a compound of Formula (IV) to give a compound of Formula (VII): 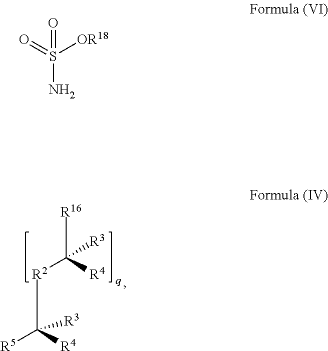
wherein
R¹⁶ is I, Br, Cl, —OH, —OSO₂CF₃, —OS(O)₂R¹³, —OP(═O)R¹³R¹⁴, —OC(═NR¹¹)R¹², —OC(═NR¹¹)CCl₃, —OR¹¹, or —N₂⁺X⁻, wherein X⁻ is halogen; and
R¹⁸ is aryl, or heteroaryl; and
b. reacting a compound of Formula (VII), and a compound of Formula (VIII) to provide the compound of Formula (VI): 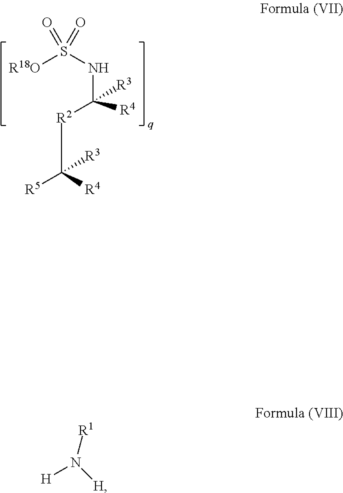
wherein
R¹ is tertiary alkyl, alkenyl, aryl, heteroaryl, carbocyclyl, or heterocyclyl.

18. The method of claim 17, wherein the compound of Formula (VII) is: 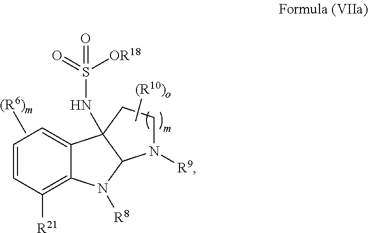
wherein
R²¹ is H, halogen, alkyl, alkenyl, alkynyl, aryl, heteroaryl, carbocyclyl, heterocyclyl, —N₃, 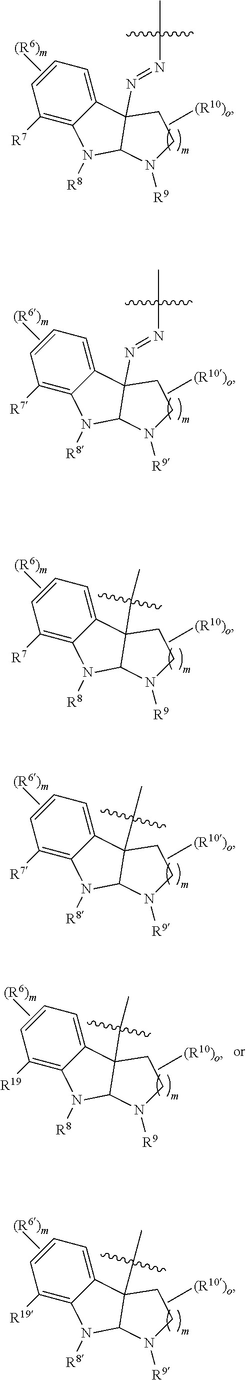
R⁷ and R^{7′} are each independently selected from H, —N₃, —N(R¹⁵)NH₂, —NHR¹⁵, 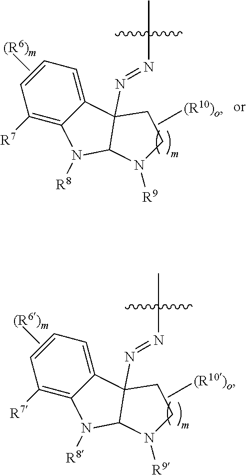
and wherein
R¹⁹ and R^{19′} are each independently selected from H, tertiary alkyl, alkenyl, aryl, heteroaryl, carbocyclyl, heterocyclyl, 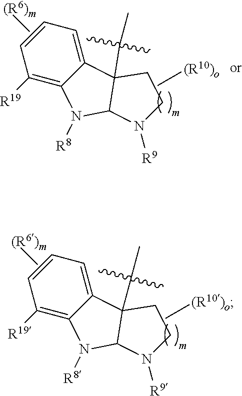
R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R^{8′}, R⁹, and R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², −SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R¹⁸ is 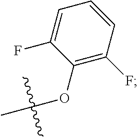 [SMILES: *Oc1c(F)cccc1F]
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

19. The method of claim 17, wherein the compound of Formula (VIII) is: 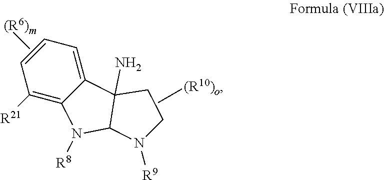
wherein
R²¹ is H, halogen, alkyl, alkenyl, alkynyl, aryl, heteroaryl, carbocyclyl, heterocyclyl, —N₃, 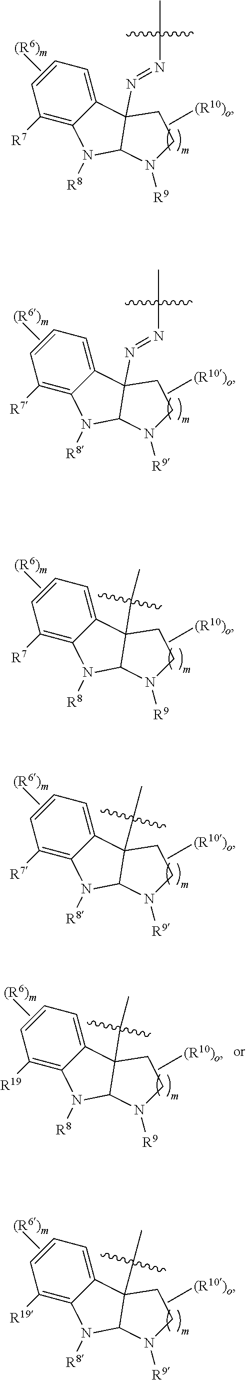
wherein
R⁷ and R^{7′} are each independently selected from H, —N₃, —N(R¹⁵)NH₂, —NHR¹⁵, 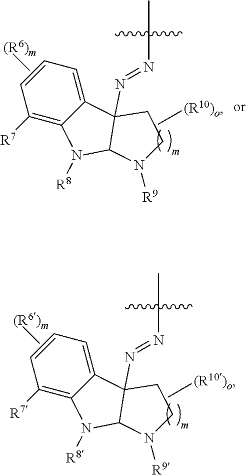
and wherein
R¹⁹ and R^{19′} are each independently selected from H, tertiary alkyl, alkenyl, aryl, heteroaryl, carbocyclyl, heterocyclyl 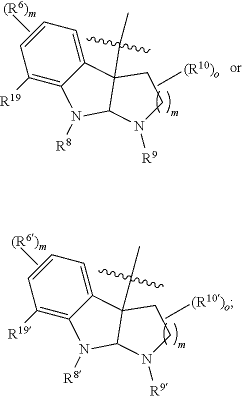
R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R^{8′}, R⁹, and R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², −SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹³, —C(═O)R²⁰, —C(═O)O(CH₂)ᵣR²⁰, —C(═O)CF₃, —C(═O)OR¹¹, —P(═O)R¹³R¹⁴, or, —P(═O)NR¹¹R¹²;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

20. The method of claim 18, wherein the compound of Formula (VIII) is: 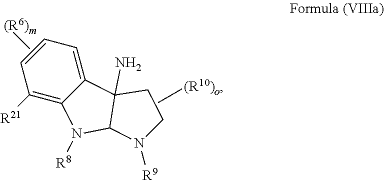
wherein
R²¹ is H, halogen, alkyl, alkenyl, alkynyl, aryl, heteroaryl, carbocyclyl, heterocyclyl, —N₃, 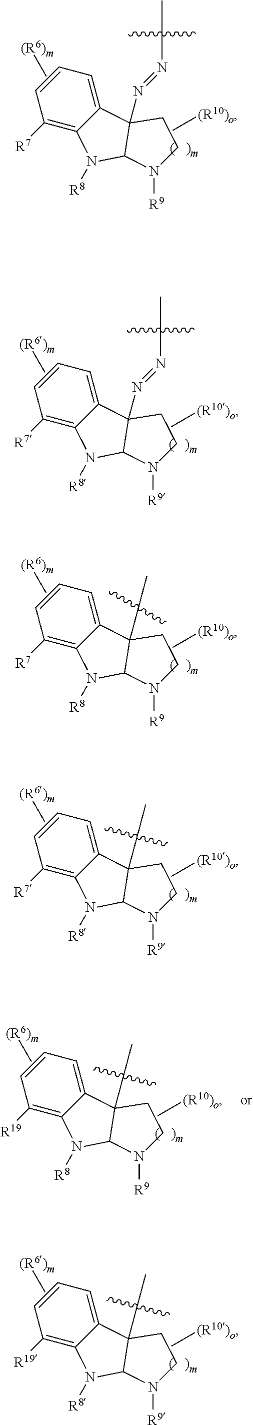
R⁷ and R^{7′} are each independently selected from H, —N₃, —N(R¹⁵)NH₂, —NHR¹⁵, 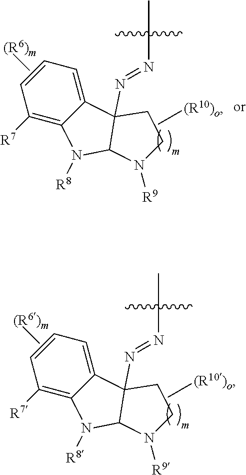
and wherein
R¹⁹ and R^{19′} are each independently selected from H, tertiary alkyl, alkenyl, aryl, heteroaryl, carbocyclyl, heterocyclyl, 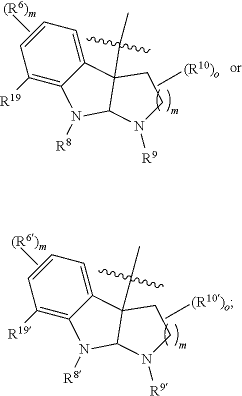
R⁶ and R^{6′} are each independently selected from halogen, —OH, —OR¹¹, —OC(═O)R¹¹, —NR¹¹R¹², —S(═O)ₚR¹³, —C(═O)R¹¹, —C(═O)OR¹¹, —C(═O)NR¹¹NR¹², C₁-C₁₂ alkyl, C₂-C₁₂ alkenyl, C₂-C₁₂ alkynyl, aryl, heteroaryl, carbocyclyl, or heterocyclyl; wherein two R⁶ or two R^{6′} groups taken together with the carbon atoms to which they are attached form an aryl, heteroaryl, carbocyclic, or heterocyclic ring;
R⁸, R^{8′}, R⁹, and R^{9′} are each independently selected from H, C₁-C₁₂ alkyl, C₂-C₁₂ alkenyl, C₂-C₁₂ alkynyl, —C(═O)R¹¹, —C(═O)OR¹¹, —C(═O)NR¹¹R¹², −SR¹¹, —S(═O)ₚR¹³, —S(═O)₂NR¹¹R¹², —C(═O)O(CH₂)ₒR¹¹, aryl, heteroaryl, carbocyclyl, or heterocyclyl;
R¹⁰ and R^{10′} are each independently selected from H, C₁-C₁₂ alkyl; C₂-C₁₂ alkenyl, C₂-C₁₂ alkynyl, —C(═O)R¹¹, —C(═O)OR¹¹, —C(═O)NR¹¹R¹², —S(═O)ₚR¹³, —OH, —OR¹¹, —OC(═O)R¹¹, —NR¹¹R¹², aryl, heteroaryl, carbocyclyl, or heterocyclyl, wherein two R¹⁰ or two R^{10′} groups taken together with the carbon atoms to which they are attached form an aryl, heteroaryl, carbocyclic, or heterocyclic ring;
R¹¹ and R¹² are each independently selected from H, C₁-C₁₂ alkyl, C₂-C₁₂ alkenyl, C₂-C₁₂ alkynyl, aryl, heteroaryl, carbocyclyl, or heterocyclyl, wherein R¹¹ and R¹² taken together with the carbon atoms to which they are attached form an aryl, heteroaryl, carbocyclic, or heterocyclic ring;
R¹³ and R¹⁴ are each independently selected from C₁-C₁₂ alkyl, C₂-C₁₂ alkenyl, C₂-C₁₂ alkynyl, aryl, heteroaryl, carbocyclyl, heterocyclyl, —OR¹¹, —(CH₂)ᵣSiMe₃, or —(CH₂)ᵣR¹¹;
R¹⁵ is —S(═O)ₚR¹³, —S(═O)₂NR¹¹R¹², —C(═O)R¹¹, —C(═O)R²⁰, —C(═O)O(CH₂)ᵣR²⁰, —C(═O)CF₃, —C(═O)OR²⁰, —P(═O)R¹³R¹⁴, or, —P(═O)NR¹¹R¹²;
R²⁰ is —Si(alkyl)₃, —Si(alkyl)₂aryl, or Si(aryl)₂alkyl; and
m is an integer from 0 to 3;
n and o are each independently an integer from 0 to 4;
p is 1 or 2; and
r is an integer from 1 to 4.

21. The method of claim 1, wherein the reaction is a radical recombination reaction.

22. The method of claim 21, wherein the reaction is carried out by irradiation.

23. The method of claim 22, wherein the irradiation occurs in a photoreactor.

24. The method of claim 23, wherein the photoreactor is equipped with 1 to about 20 lamps operating at a wavelength λ from about 250 nm to about 400 nm.

25. The method of claim 24 wherein the wavelength λ is 300 nm or 380 nm.

26. The method of claim 21, wherein the radical recombination reaction results in the formation of a Csp3-Csp3 bond or a Csp3-Csp2 bond.

27. The method of claim 1, wherein the stereochemical configuration of the compound of Formula (II) is retained in the compound of Formula (I) following the reaction.

28. The method of claim 7, wherein the reaction comprises an electrophilic activation of a compound of Formula (IV).

29. The method of claim 28, wherein the electrophilic activation comprises reaction of the compound of Formula (IV) with a silver (I) salt, and a base.

30. The method of claim 29, wherein the silver (I) salt is AgOSO₂CF₃ or AgSF₆.

31. The method of claim 29, wherein the compound of Formula (III) is resistant to oxidation by the silver (I) salt.

32. The method of claim 15, wherein the sulfur dioxide extrusion is carried out in the presence of an oxidizing agent.

33. The method of claim 32, wherein the oxidizing agent is an electrophilic halogenating reagent.

34. The method of claim 1, wherein the compounds of Formula (I) are: 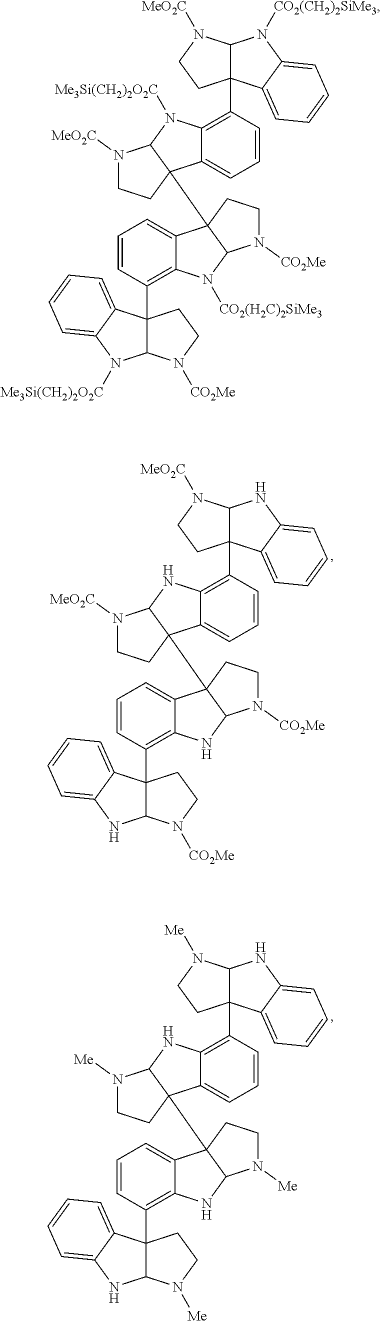 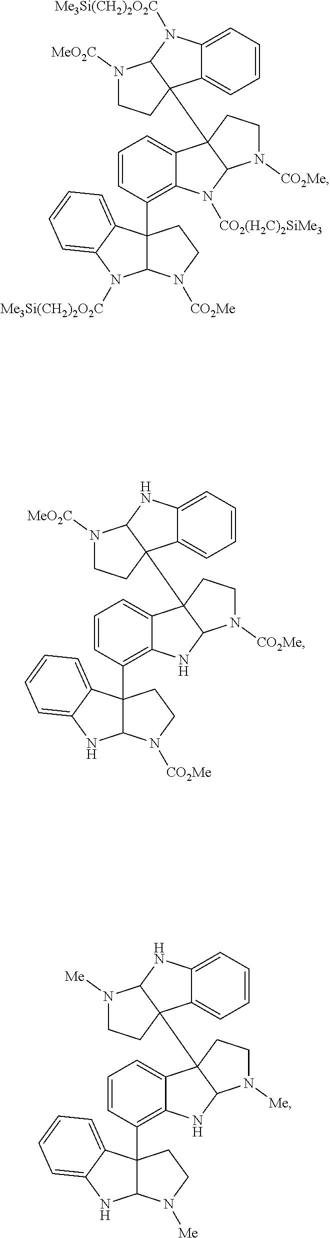 [chemical structure]
